SEQUENCE LISTING (432 sequences)

SEQ ID NO: 1 - protein, 108 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIAFNDYKLSFGAGTTVTVRA

SEQ ID NO: 2 - protein, 108 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIAYNDYKLSFGAGTTVTVRA

SEQ ID NO: 3 - protein, 110 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVFGGSQGNLIFGKGTKLSVKP

SEQ ID NO: 4 - protein, 108 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVFNDYKLSFGAGTTVTVRA

SEQ ID NO: 5 - protein, 110 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVYGGSQGNLIFGKGTKLSVKP

SEQ ID NO: 6 - protein, 108 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVYNDYKLSFGAGTTVTVRA

SEQ ID NO: 7 - protein, 110 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGPYNTDKLIFGTGTRLQVFP

SEQ ID NO: 8 - protein, 111 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGVMEYGNKLVFGAGTILRVKS

SEQ ID NO: 9 - protein, 114 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSAGQGGTGELFFGEGSRLTVL

SEQ ID NO: 10 - protein, 111 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSFDGETQYFGPGTRLLVL

SEQ ID NO: 11 - protein, 114 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLGQPSTDTQYFGPGTRLTVL

SEQ ID NO: 12 - protein, 114 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASFLGPVFPGGYTFGSGTRLTVV

SEQ ID NO: 13 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLVGWETQYFGPGTRLLVL

SEQ ID NO: 14 - protein, 113 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSLNWDTEAFFGQGTRLTVV

SEQ ID NO: 15 - protein, 116 aa, Homo sapiens
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSLASAGGTDTQYFGPGTRLTVL

SEQ ID NO: 16 - protein, 113 aa, Homo sapiens
GAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLNWDTEAFFGQGTRLTVV

SEQ ID NO: 17 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSFRHTDTQYFGPGTRLTVL

SEQ ID NO: 18 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSFRSTDTQYFGPGTRLTVL

SEQ ID NO: 19 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSFRTTDTQYFGPGTRLTVL

SEQ ID NO: 20 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSFRYTDTQYFGPGTRLTVL

SEQ ID NO: 21 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSIRATDTQYFGPGTRLTVL

SEQ ID NO: 22 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSIRDTDTQYFGPGTRLTVL

SEQ ID NO: 23 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSIRFTDTQYFGPGTRLTVL

SEQ ID NO: 24 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSIRGTDTQYFGPGTRLTVL

SEQ ID NO: 25 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSIRHTDTQYFGPGTRLTVL

SEQ ID NO: 26 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSIRLTDTQYFGPGTRLTVL

SEQ ID NO: 27 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSIRSTDTQYFGPGTRLTVL

SEQ ID NO: 28 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSIRVTDTQYFGPGTRLTVL

SEQ ID NO: 29 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSIRYTDTQYFGPGTRLTVL

SEQ ID NO: 30 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRATDTQYFGPGTRLTVL

SEQ ID NO: 31 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRFTDTQYFGPGTRLTVL

SEQ ID NO: 32 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRHTDTQYFGPGTRLTVL

SEQ ID NO: 33 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRSTDTQYFGPGTRLTVL

SEQ ID NO: 34 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRWTDTQYFGPGTRLTVL

SEQ ID NO: 35 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRYTDTQYFGPGTRLTVL

SEQ ID NO: 36 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSVRFTDTQYFGPGTRLTVL

SEQ ID NO: 37 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSVRSTDTQYFGPGTRLTVL

SEQ ID NO: 38 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSVRYTDTQYFGPGTRLTVL

SEQ ID NO: 39 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSYRSTDTQYFGPGTRLTVL

SEQ ID NO: 40 - protein, 113 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSFRSTDTQYFGPGTRLTVL

SEQ ID NO: 41 - protein, 113 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSIRATDTQYFGPGTRLTVL

SEQ ID NO: 42 - protein, 113 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSIRGTDTQYFGPGTRLTVL

SEQ ID NO: 43 - protein, 113 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSIRSTDTQYFGPGTRLTVL

SEQ ID NO: 44 - protein, 113 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSLRATDTQYFGPGTRLTVL

SEQ ID NO: 45 - protein, 113 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSLRHTDTQYFGPGTRLTVL

SEQ ID NO: 46 - protein, 113 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSLRSTDTQYFGPGTRLTVL

SEQ ID NO: 47 - protein, 113 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSVRATDTQYFGPGTRLTVL

SEQ ID NO: 48 - protein, 113 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSVRSTDTQYFGPGTRLTVL

SEQ ID NO: 49 - protein, 113 aa, Homo sapiens
  misc_feature (96)..(96): Xaa can be any naturally occurring amino acid
  misc_feature (98)..(98): Xaa can be any naturally occurring amino acid
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSXRXTDTQYFGPGTRLTVL

SEQ ID NO: 50 - protein, 113 aa, Homo sapiens
  misc_feature (96)..(96): Xaa can be any naturally occurring amino acid
  misc_feature (98)..(98): Xaa can be any naturally occurring amino acid
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSXRXTDTQYFGPGTRLTVL

SEQ ID NO: 51 - protein, 113 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCAVRAVFSGGYNKLIFGAGTRLAVHP

SEQ ID NO: 52 - protein, 113 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCAVRAVLSGGYNKLIFGAGTRLAVHP

SEQ ID NO: 53 - protein, 113 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCAVRAVVSGGYNKLIFGAGTRLAVHP

SEQ ID NO: 54 - protein, 110 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCAVTSSNTGKLIFGQGTTLQVKP

SEQ ID NO: 55 - protein, 110 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCAVTTSNTGKLIFGQGTTLQVKP

SEQ ID NO: 56 - protein, 111 aa, Homo sapiens
RKEVEQDPGPFNVPEGATVAFNCTYSNSASQSFFWYRQDCRKEPKLLMSVYSSGNEDGRFTAQLNRASQYISLLIRDSKLSDSATYLCVVNLYSSASKIIFGSGTRLSIRP

SEQ ID NO: 57 - protein, 111 aa, Homo sapiens
RKEVEQDPGPFNVPEGATVAFNCTYSNSASQSFFWYRQDCRKEPKLLMSVYSSGNEDGRFTAQLNRASQYISLLIRDSKLSDSATYLCVVNNASSASKIIFGSGTRLSIRP

SEQ ID NO: 58 - protein, 111 aa, Homo sapiens
RKEVEQDPGPFNVPEGATVAFNCTYSNSASQSFFWYRQDCRKEPKLLMSVYSSGNEDGRFTAQLNRASQYISLLIRDSKLSDSATYLCVVNQYSSASKIIFGSGTRLSIRP

SEQ ID NO: 59 - protein, 111 aa, Homo sapiens
RKEVEQDPGPFNVPEGATVAFNCTYSNSASQSFFWYRQDCRKEPKLLMSVYSSGNEDGRFTAQLNRASQYISLLIRDSKLSDSATYLCVVNSASSASKIIFGSGTRLSIRP

SEQ ID NO: 60 - protein, 112 aa, Homo sapiens
RKEVEQDPGPFNVPEGATVAFNCTYSNSASQSFFWYRQDCRKEPKLLMSVYSSGNEDGRFTAQLNRASQYISLLIRDSKLSDSATYLCVVTLMDTGRRALTFGSGTRLQVQP

SEQ ID NO: 61 - protein, 110 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAPQGATNKLIFGTGTLLAVQP

SEQ ID NO: 62 - protein, 111 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCASQDTGRRALTFGSGTRLQVQP

SEQ ID NO: 63 - protein, 111 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVATYNFNKFYFGSGTKLNVKP

SEQ ID NO: 64 - protein, 112 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVFPGGATNKLIFGTGTLLAVQP

SEQ ID NO: 65 - protein, 111 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVKDSSASKIIFGSGTRLSIRP

SEQ ID NO: 66 - protein, 113 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVNMFSGGYNKLIFGAGTRLAVHP

SEQ ID NO: 67 - protein, 114 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVNMNYGGATNKLIFGTGTLLAVQP

SEQ ID NO: 68 - protein, 110 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVPNRDDKIIFGKGTRLHILP

SEQ ID NO: 69 - protein, 110 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVSNRDDKIIFGKGTRLHILP

SEQ ID NO: 70 - protein, 111 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAAPQGGSEKLVFGKGTKLTVNP

SEQ ID NO: 71 - protein, 111 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAIYTGTASKLTFGTGTRLQVTL

SEQ ID NO: 72 - protein, 111 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMIEAAGNKLTFGGGTRVLVKP

SEQ ID NO: 73 - protein, 111 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMIQAAGNKLTFGGGTRVLVKP

SEQ ID NO: 74 - protein, 110 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMKDYGQNFVFGPGTRLSVLP

SEQ ID NO: 75 - protein, 111 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMLEAAGNKLTFGGGTRVLVKP

SEQ ID NO: 76 - protein, 111 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMNDYGNNRLAFGKGNQVVVIP

SEQ ID NO: 77 - protein, 110 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMRDYGQNFVFGPGTRLSVLP

SEQ ID NO: 78 - protein, 111 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSAGTGNQFYFGTGTSLTVIP

SEQ ID NO: 79 - protein, 114 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSASSGGGADGLTFGKGTHLIIQP

SEQ ID NO: 80 - protein, 114 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSDLPGGSNYKLTFGKGTLLTVNP

SEQ ID NO: 81 - protein, 111 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSEAAGNKLTFGGGTRVLVKP

SEQ ID NO: 82 - protein, 111 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSEGTGNQFYFGTGTSLTVIP

SEQ ID NO: 83 - protein, 114 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSEIPGGSNYKLTFGKGTLLTVNP

SEQ ID NO: 84 - protein, 114 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSELPGGSNYKLTFGKGTLLTVNP

SEQ ID NO: 85 - protein, 111 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMTDYGNNRLAFGKGNQVVVIP

SEQ ID NO: 86 - protein, 110 aa, Homo sapiens
GENVEQHPSTLSVQEGDSAVIKCTYSDSASNYFPWYKQELGKGPQLIIDIRSNVGEKKDQRIAVTLNKTAKHFSLHITETQPEDSAVYFCAASNTDKLIFGTGTRLQVFP

SEQ ID NO: 87 - protein, 115 aa, Homo sapiens
GESVGLHLPTLSVQEGDNSIINCAYSNSASDYFIWYKQESGKGPQFIIDIRSNMDKRQGQRVTVLLNKTVKHLSLQIAATQPGDSAVYFCAEGDAGGTSYGKLTFGQGTILTVHP

SEQ ID NO: 88 - protein, 115 aa, Homo sapiens
GESVGLHLPTLSVQEGDNSIINCAYSNSASDYFIWYKQESGKGPQFIIDIRSNMDKRQGQRVTVLLNKTVKHLSLQIAATQPGDSAVYFCAETNAGGTSYGKLTFGQGTILTVHP

SEQ ID NO: 89 - protein, 113 aa, Homo sapiens
AQKITQTQPGMFVQEKEAVTLDCTYDTSDPSYGLFWYKQPSSGEMIFLIYQGSYDQQNATEGRYSLNFQKARKSANLVISASQLGDSAMYFCAMNTGGFKTIFGAGTRLFVKA

SEQ ID NO: 90 - protein, 115 aa, Homo sapiens
AQKITQTQPGMFVQEKEAVTLDCTYDTSDPSYGLFWYKQPSSGEMIFLIYQGSYDQQNATEGRYSLNFQKARKSANLVISASQLGDSAMYFCAMREEGSQGNLIFGKGTKLSVKP

SEQ ID NO: 91 - protein, 117 aa, Homo sapiens
AQKITQTQPGMFVQEKEAVTLDCTYDTSDPSYGLFWYKQPSSGEMIFLIYQGSYDQQNATEGRYSLNFQKARKSANLVISASQLGDSAMYFCAMREGRYSSASKIIFGSGTRLSIRP

SEQ ID NO: 92 - protein, 109 aa, Homo sapiens
AQRVTQPEKLLSVFKGAPVELKCNYSYSGSPELFWYVQYSRQRLQLLLRHISRESIKGFTADLNKGETSFHLKKPFAQEEDSAMYYCALNSGGYQKVTFGIGTKLQVIP

SEQ ID NO: 93 - protein, 109 aa, Homo sapiens
AQRVTQPEKLLSVFKGAPVELKCNYSYSGSPELFWYVQYSRQRLQLLLRHISRESIKGFTADLNKGETSFHLKKPFAQEEDSAMYYCALSAPINYQLIWGAGTKLIIKP

SEQ ID NO: 94 - protein, 108 aa, Homo sapiens
AQRVTQPEKLLSVFKGAPVELKCNYSYSGSPELFWYVQYSRQRLQLLLRHISRESIKGFTADLNKGETSFHLKKPFAQEEDSAMYYCALSDSNYQLIWGAGTKLIIKP

SEQ ID NO: 95 - protein, 112 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDAETSGSRLTFGEGTQLTVNP

SEQ ID NO: 96 - protein, 112 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDDKGGSEKLVFGKGTKLTVNP

SEQ ID NO: 97 - protein, 112 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATEGNTGFQKLVFGTGTRLLVSP

SEQ ID NO: 98 - protein, 120 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAFGAGGTSYGKLTFGQGTILTVHP

SEQ ID NO: 99 - protein, 115 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGANSKLTFGKGITLSVRP

SEQ ID NO: 100 - protein, 121 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEGGFGNVLHFGNVLHCGSGTQVIVLP

SEQ ID NO: 101 - protein, 116 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEGGNAGNMLTFGGGTRLMVKP

SEQ ID NO: 102 - protein, 116 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEGGNQGGKLIFGQGTELSVKP

SEQ ID NO: 103 - protein, 116 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEGSNAGNMLTFGGGTRLMVKP

SEQ ID NO: 104 - protein, 115 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSGAGANSKLTFGKGITLSVRP

SEQ ID NO: 105 - protein, 115 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSGGGANSKLTFGKGITLSVRP

SEQ ID NO: 106 - protein, 114 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALTLNRDDKIIFGKGTRLHILP

SEQ ID NO: 107 - protein, 108 aa, Homo sapiens
KDQVFQPSTVASSEGAVVEIFCNHSVSNAYNFFWYLHFPGCAPRLLVKGSKPSQQGRYNMTYERFSSSLLILQVREADAAVYYCAVEDLRAGSYQLTFGKGTKLSVIP

SEQ ID NO: 108 - protein, 106 aa, Homo sapiens
KDQVFQPSTVASSEGAVVEIFCNHSVSNAYNFFWYLHFPGCAPRLLVKGSKPSQQGRYNMTYERFSSSLLILQVREADAAVYYCAVEVYNFNKFYFGSGTKLNVKP

SEQ ID NO: 109 - protein, 114 aa, Homo sapiens
EDQVTQSPEALRLQEGESSSLNCSYTVSGLRGLFWYRQDPGKGPEFLFTLYSAGEEKEKERLKATLTKKESFLHITAPKPEDSATYLCAVQGDRLTGGGNKLTFGTGTQLKVEL

SEQ ID NO: 110 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVPSGAGSYQLTFGKGTKLSVIP

SEQ ID NO: 111 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVTGTYKYIFGTGTRLKVLA

SEQ ID NO: 112 - protein, 109 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVELQGAQKLVFGQGTRLTINP

SEQ ID NO: 113 - protein, 110 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVERADSWGKLQFGAGTQVVVTP

SEQ ID NO: 114 - protein, 109 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVERQGAQKLVFGQGTRLTINP

SEQ ID NO: 115 - protein, 115 aa, Homo sapiens
QQQVKQSPQSLIVQKGGISIINCAYENTAFDYFPWYQQFPGKGPALLIAIRPDVSEKKEGRFTISFNKSAKQFSLHIMDSQPGDSATYFCAASSAGGTSYGKLTFGQGTILTVHP

SEQ ID NO: 116 - protein, 109 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIAPSGTYKYIFGTGTRLKVLA

SEQ ID NO: 117 - protein, 111 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIDPGSSNTGKLIFGQGTTLQVKP

SEQ ID NO: 118 - protein, 111 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIGNYGGSQGNLIFGKGTKLSVKP

SEQ ID NO: 119 - protein, 111 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIPNYGGSQGNLIFGKGTKLSVKP

SEQ ID NO: 120 - protein, 108 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCISFNDYKLSFGAGTTVTVRA

SEQ ID NO: 121 - protein, 107 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVFNARLMFGDGTQLVVKP

SEQ ID NO: 122 - protein, 107 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVHNARLMFGDGTQLVVKP

SEQ ID NO: 123 - protein, 110 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVLGGATNKLIFGTGTLLAVQP

SEQ ID NO: 124 - protein, 107 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVLNARLMFGDGTQLVVKP

SEQ ID NO: 125 - protein, 110 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVPPGTASKLTFGTGTRLQVTL

SEQ ID NO: 126 - protein, 109 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVPQGAQKLVFGQGTRLTINP

SEQ ID NO: 127 - protein, 110 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVRVVGDDKIIFGKGTRLHILP

SEQ ID NO: 128 - protein, 108 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVTDGQKLLFARGTMLKVDL

SEQ ID NO: 129 - protein, 107 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVTGNQFYFGTGTSLTVIP

SEQ ID NO: 130 - protein, 108 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVTSGSRLTFGEGTQLTVNP

SEQ ID NO: 131 - protein, 109 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVYGGSEKLVFGKGTKLTVNP

SEQ ID NO: 132 - protein, 107 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVYNARLMFGDGTQLVVKP

SEQ ID NO: 133 - protein, 107 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVYNNDMRFGAGTRLTVKP

SEQ ID NO: 134 - protein, 108 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVYNTDKLIFGTGTRLQVFP

SEQ ID NO: 135 - protein, 109 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVYSGNTPLVFGKGTRLSVIA

SEQ ID NO: 136 - protein, 115 aa, Homo sapiens
TQLLEQSPQFLSIQEGENLTVYCNSSSVFSSLQWYRQEPGEGPVLLVTVVTGGEVKKLKRLTFQFGDARKDSSLHITAAQPGDTGLYLCAGEGNAGGTSYGKLTFGQGTILTVHP

SEQ ID NO: 137 - protein, 116 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAASADAGGTSYGKLTFGQGTILTVHP

SEQ ID NO: 138 - protein, 114 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAASAGETSGSRLTFGEGTQLTVNP

SEQ ID NO: 139 - protein, 114 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAASALTSGTYKYIFGTGTRLKVLA

SEQ ID NO: 140 - protein, 113 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAASEETSGSRLTFGEGTQLTVNP

SEQ ID NO: 141 - protein, 115 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAASEQSGGSNYKLTFGKGTLLTVNP

SEQ ID NO: 142 - protein, 115 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAASGGGGSTLGRLYFGRGTQLTVWP

SEQ ID NO: 143 - protein, 114 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAASVATDSWGKLQFGAGTQVVVTP

SEQ ID NO: 144 - protein, 116 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAASVLYGSSNTGKLIFGQGTTLQVKP

SEQ ID NO: 145 - protein, 112 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAATNTNAGKSTFGDGTTLTVKP

SEQ ID NO: 146 - protein, 114 aa, Homo sapiens
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDGYGNNRLAFGKGNQVVVIP

SEQ ID NO: 147 - protein, 106 aa, Homo sapiens
AQSVAQPEDQVNVAEGNPLTVKCTYSVSGNPYLFWYVQYPNRGLQFLLKYITGDNLVKGSYGFEAEFNKSQTSFHLKKPSALVSDSALYFCRTLTFGKGTMLLVSP

SEQ ID NO: 148 - protein, 110 aa, Homo sapiens
SQELEQSPQSLIVQEGKNLTINCTSSKTLYGLYWYKQKYGEGLIFLMMLQKGGEEKSHEKITAKLDEKKQQSSLHITASQPSHAGIYLCGADQGAQKLVFGQGTRLTINP

SEQ ID NO: 149 - protein, 108 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAANDYKLSFGAGTTVTVRA

SEQ ID NO: 150 - protein, 112 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAATTGGSQGNLIFGKGTKLSVKP

SEQ ID NO: 151 - protein, 112 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGDSGGGADGLTFGKGTHLIIQP

SEQ ID NO: 152 - protein, 109 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGDSNYQLIWGAGTKLIIKP

SEQ ID NO: 153 - protein, 109 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGFNTDKLIFGTGTRLQVFP

SEQ ID NO: 154 - protein, 109 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGGNDYKLSFGAGTTVTVRA

SEQ ID NO: 155 - protein, 109 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGHNTDKLIFGTGTRLQVFP

SEQ ID NO: 156 - protein, 108 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGNDYKLSFGAGTTVTVRA

SEQ ID NO: 157 - protein, 112 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGNYGGATNKLIFGTGTLLAVQP

SEQ ID NO: 158 - protein, 112 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGQLDSGTYKYIFGTGTRLKVLA

SEQ ID NO: 159 - protein, 112 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGQLGGATNKLIFGTGTLLAVQP

SEQ ID NO: 160 - protein, 115 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGQLNAGGTSYGKLTFGQGTILTVHP

SEQ ID NO: 161 - protein, 113 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGQPGSSNTGKLIFGQGTTLQVKP

SEQ ID NO: 162 - protein, 110 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGQQGAQKLVFGQGTRLTINP

SEQ ID NO: 163 - protein, 113 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGQVGSSNTGKLIFGQGTTLQVKP

SEQ ID NO: 164 - protein, 110 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFCAGVYNNNDMRFGAGTRLTVKP

SEQ ID NO: 165 - protein, 116 aa, Homo sapiens
AQTVTQSQPEMSVQEAETVTLSCTYDTSENNYYLFWYKQPPSRQMILVIRQEAYKQQNATENRFSVNFQKAAKSFSLKISDSQLGDTAMYFCAFTVYTGANSKLTFGKGITLSVRP

SEQ ID NO: 166 - protein, 116 aa, Homo sapiens
AQTVTQSQPEMSVQEAETVTLSCTYDTSESDYYLFWYKQPPSRQMILVIRQEAYKQQNATENRFSVNFQKAAKSFSLKISDSQLGDAAMYFCAYRSTRYNNNDMRFGAGTRLTVKP

SEQ ID NO: 167 - protein, 115 aa, Homo sapiens
AQTVTQSQPEMSVQEAETVTLSCTYDTSESDYYLFWYKQPPSRQMILVIRQEAYKQQNATENRFSVNFQKAAKSFSLKISDSQLGDAAMYFCAYRTTRYGQNFVFGPGTRLSVLP

SEQ ID NO: 168 - protein, 109 aa, Homo sapiens
ELKVEQNPLFLSMQEGKNYTIYCNYSTTSDRLYWYRQDPGKSLESLFVLLSNGAVKQEGRLMASLDTKARLSTLHITAAVHDLSATYFCAVDPGYALNFGKGTSLLVTP

SEQ ID NO: 169 - protein, 109 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVDNAGNMLTFGGGTRLMVKP

SEQ ID NO: 170 - protein, 111 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGDDTGFQKLVFGTGTRLLVSP

SEQ ID NO: 171 - protein, 113 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGDENTGTASKLTFGTGTRLQVTL

SEQ ID NO: 172 - protein, 112 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGDETGGYNKLIFGAGTRLAVHP

SEQ ID NO: 173 - protein, 113 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGDGDGGATNKLIFGTGTLLAVQP

SEQ ID NO: 174 - protein, 111 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGDGGGYNKLIFGAGTRLAVHP

SEQ ID NO: 175 - protein, 111 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGDPTGFQKLVFGTGTRLLVSP

SEQ ID NO: 176 - protein, 111 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGEGDSNYQLIWGAGTKLIIKP

SEQ ID NO: 177 - protein, 111 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGGAGGYNKLIFGAGTRLAVHP

SEQ ID NO: 178 - protein, 111 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGGDNQGGKLIFGQGTELSVKP

SEQ ID NO: 179 - protein, 110 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGGDSSYKLIFGSGTRLLVRP

SEQ ID NO: 180 - protein, 111 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGGGGGADGLTFGKGTHLIIQP

SEQ ID NO: 181 - protein, 113 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGGHGSSNTGKLIFGQGTTLQVKP

SEQ ID NO: 182 - protein, 111 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGGSGGYNKLIFGAGTRLAVHP

SEQ ID NO: 183 - protein, 110 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGGYNNNDMRFGAGTRLTVKP

SEQ ID NO: 184 - protein, 118 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGQNFGNEKLTFGNEKLTFGTGTRLTIIP

SEQ ID NO: 185 - protein, 112 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGTLTGGGNKLTFGTGTQLKVEL

SEQ ID NO: 186 - protein, 108 aa, Homo sapiens
KNEVEQSPQNLTAQEGEFITINCSYSVGISALHWLQQHPGGGIVSLFMLSSGKKKHGRLIATINIQEKHSSLHITASHPRDSAVYICAVAGTASKLTFGTGTRLQVTL

SEQ ID NO: 187 - protein, 109 aa, Homo sapiens
KNEVEQSPQNLTAQEGEFITINCSYSVGISALHWLQQHPGGGIVSLFMLSSGKKKHGRLIATINIQEKHSSLHITASHPRDSAVYICAVEAGSNYQLIWGAGTKLIIKP

SEQ ID NO: 188 - protein, 109 aa, Homo sapiens
KNEVEQSPQNLTAQEGEFITINCSYSVGISALHWLQQHPGGGIVSLFMLSSGKKKHGRLIATINIQEKHSSLHITASHPRDSAVYICAVEGGSNYKLTFGKGTLLTVNP

SEQ ID NO: 189 - protein, 109 aa, Homo sapiens
KNEVEQSPQNLTAQEGEFITINCSYSVGISALHWLQQHPGGGIVSLFMLSSGKKKHGRLIATINIQEKHSSLHITASHPRDSAVYICAVESGSNYQLIWGAGTKLIIKP

SEQ ID NO: 190 - protein, 108 aa, Homo sapiens
KNEVEQSPQNLTAQEGEFITINCSYSVGISALHWLQQHPGGGIVSLFMLSSGKKKHGRLIATINIQEKHSSLHITASHPRDSAVYICAVETSGSRLTFGEGTQLTVNP

SEQ ID NO: 191 - protein, 109 aa, Homo sapiens
KNEVEQSPQNLTAQEGEFITINCSYSVGISALHWLQQHPGGGIVSLFMLSSGKKKHGRLIATINIQEKHSSLHITASHPRDSAVYICAVEWGSNYQLIWGAGTKLIIKP

SEQ ID NO: 192 - protein, 110 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAEAGGGNKLTFGTGTQLKVEL

SEQ ID NO: 193 - protein, 112 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAESKSGGYNKLIFGAGTRLAVHP

SEQ ID NO: 194 - protein, 110 aa, Homo sapiens
SQKIEQNSEALNIQEGKTATLTCNYTNYSPAYLQWYRQDPGRGPVFLLLIRENEKEKRKERLKVTFDTTLKQSLFHITASQPADSATYLCALPSGYALNFGKGTSLLVTP

SEQ ID NO: 195 - protein, 112 aa, Homo sapiens
SQKIEQNSEALNIQEGKTATLTCNYTNYSPAYLQWYRQDPGRGPVFLLLIRENEKEKRKERLKVTFDTTLKQSLFHITASQPADSATYLCALSTDSWGKLQFGAGTQVVVTP

SEQ ID NO: 196 - protein, 114 aa, Homo sapiens
AQSVSQHNHHVILSEAASLELGCNYSYGGTVNLFWYVQYPGQHLQLLLKYFSGDPLVKGIKGFEAEFIKSKFSFNLRKPSVQWSDTAEYFCAVNARNAGNMLTFGGGTRLMVKP

SEQ ID NO: 197 - protein, 114 aa, Homo sapiens
AQSVSQHNHHVILSEAASLELGCNYSYGGTVNLFWYVQYPGQHLQLLLKYFSGDPLVKGIKGFEAEFIKSKFSFNLRKPSVQWSDTAEYFCAVNARNSGYALNFGKGTSLLVTP

SEQ ID NO: 198 - protein, 114 aa, Homo sapiens
AQSVSQHNHHVILSEAASLELGCNYSYGGTVNLFWYVQYPGQHLQLLLKYFSGDPLVKGIKGFEAEFIKSKFSFNLRKPSVQWSDTAEYFCAVNRNTGFQKLVFGTGTRLLVSP

SEQ ID NO: 199 - protein, 121 aa, Homo sapiens
AQSVTQLDSHVSVSEGTPVLLRCNYSSSYSPSLFWYVQHPNKGLQLLLKYTSAATLVKGINGFEAEFKKSETSFHLTKPSAHMSDAAEYFCASLSNFGNEKLTFGNEKLTFGTGTRLTIIP

SEQ ID NO: 200 - protein, 113 aa, Homo sapiens
AQSVTQLDSHVSVSEGTPVLLRCNYSSSYSPSLFWYVQHPNKGLQLLLKYTSAATLVKGINGFEAEFKKSETSFHLTKPSAHMSDAAEYFCAVSEWAGNQFYFGTGTSLTVIP

SEQ ID NO: 201 - protein, 116 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVATDRGSTLGRLYFGRGTQLTVWP

SEQ ID NO: 202 - protein, 114 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGAAEYGNKLVFGAGTILRVKS

SEQ ID NO: 203 - protein, 114 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGASEYGNKLVFGAGTILRVKS

SEQ ID NO: 204 - protein, 114 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGAVEYGNKLVFGAGTILRVKS

SEQ ID NO: 205 - protein, 116 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGLDRGSTLGRLYFGRGTQLTVWP

SEQ ID NO: 206 - protein, 114 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGLTDSWGKLQFGAGTQVVVTP

SEQ ID NO: 207 - protein, 114 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGPAEYGNKLVFGAGTILRVKS

SEQ ID NO: 208 - protein, 116 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGSDRGSTLGRLYFGRGTQLTVWP

SEQ ID NO: 209 - protein, 116 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGTDRGSTLGRLYFGRGTQLTVWP

SEQ ID NO: 210 - protein, 116 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGVDRGSTLGRLYFGRGTQLTVWP

SEQ ID NO: 211 - protein, 114 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGVSEYGNKLVFGAGTILRVKS

SEQ ID NO: 212 - protein, 112 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVVHSSYKLIFGSGTRLLVRP

SEQ ID NO: 213 - protein, 112 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALAEYNFNKFYFGSGTKLNVKP

SEQ ID NO: 214 - protein, 115 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALSDGSGAGSYQLTFGKGTKLSVIP

SEQ ID NO: 215 - protein, 114 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALSDPTGANSKLTFGKGITLSVRP

SEQ ID NO: 216 - protein, 114 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALSDPTGTASKLTFGTGTRLQVTL

SEQ ID NO: 217 - protein, 114 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALSDQDTGRRALTFGSGTRLQVQP

SEQ ID NO: 218 - protein, 113 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALSDQTGANNLFFGTGTRLTVIP

SEQ ID NO: 219 - protein, 114 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALSDQTGTASKLTFGTGTRLQVTL

SEQ ID NO: 220 - protein, 112 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALSEGNFNKFYFGSGTKLNVKP

SEQ ID NO: 221 - protein, 113 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALSGGTSYGKLTFGQGTILTVHP

SEQ ID NO: 222 - protein, 116 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALSGSAGGTSYGKLTFGQGTILTVHP

SEQ ID NO: 223 - protein, 111 aa, Homo sapiens
DAGITQSPRHKVTETGTPVTLRCHQTENHRYMYWYRQDPGHGLRLIHYSYGVKDTDKGEVSDGYSVSRSKTEDFLLTLESATSSQTSVYFCAISASGTEAFFGQGTRLTVV

SEQ ID NO: 224 - protein, 113 aa, Homo sapiens
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSSTAQETQYFGPGTRLLVL

SEQ ID NO: 225 - protein, 114 aa, Homo sapiens
DAGVIQSPRHEVTEMGQEVTLRCKPISGHNSLFWYRQTMMRGLELLIYFNNNVPIDDSGMPEDRFSAKMPNASFSTLKIQPSEPRDSAVYFCASRLTLGTDTQYFGPGTRLTVL

SEQ ID NO: 226 - protein, 115 aa, Homo sapiens
DAGVIQSPRHEVTEMGQEVTLRCKPISGHNSLFWYRQTMMRGLELLIYFNNNVPIDDSGMPEDRFSAKMPNASFSTLKIQPSEPRDSAVYFCASRPRGAPSYEQYFGPGTRLTVT

SEQ ID NO: 227 - protein, 113 aa, Homo sapiens
DAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSWTSWDTQYFGPGTRLTVL

SEQ ID NO: 228 - protein, 114 aa, Homo sapiens
DAMVIQNPRYQVTQFGKPVTLSCSQTLNHNVMYWYQQKSSQAPKLLFHYYDKDFNNEADTPDNFQSRRPNTSFCFLDIRSPGLGDTAMYLCATSRAGGGGEKLFFGSGTQLSVL

SEQ ID NO: 229 - protein, 114 aa, Homo sapiens
NAGVMQNPRHLVRRRGQEARLRCSPMKGHSHVYWYRQLPEEGLKFMVYLQKENIIDESGMPKERFSAEFPKEGPSILRIQQVVRGDSAAYFCASSLAGWDTEAFFGQGTRLTVV

SEQ ID NO: 230 - protein, 114 aa, Homo sapiens
NAGVMQNPRHLVRRRGQEARLRCSPMKGHSHVYWYRQLPEEGLKFMVYLQKENIIDESGMPKERFSAEFPKEGPSILRIQQVVRGDSAAYFCASSPAGWDTEAFFGQGTRLTVV

SEQ ID NO: 231 - protein, 112 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCAISTQGGNEQFFGPGTRLTVL

SEQ ID NO: 232 - protein, 117 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIFSLAGASYNEQFFGPGTRLTVL

SEQ ID NO: 233 - protein, 113 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIGTSGETQYFGPGTRLLVL

SEQ ID NO: 234 - protein, 113 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIRTGGSEQYFGPGTRLTVT

SEQ ID NO: 235 - protein, 114 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIVGGADQPQHFGDGTRLSIL

SEQ ID NO: 236 - protein, 115 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIVGSGGYNEQFFGPGTRLTVL

SEQ ID NO: 237 - protein, 113 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSTGTSGETQYFGPGTRLLVL

SEQ ID NO: 238 - protein, 114 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSAESGYNEQFFGPGTRLTVL

SEQ ID NO: 239 - protein, 118 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSAKPPTGDFSYEQYFGPGTRLTVT

SEQ ID NO: 240 - protein, 115 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARGAGDSPLHFGNGTRLTVT

SEQ ID NO: 241 - protein, 115 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARRQADQPQHFGDGTRLSIL

SEQ ID NO: 242 - protein, 114 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARVWNTEAFFGQGTRLTVV

SEQ ID NO: 243 - protein, 116 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSASAGTFTDTQYFGPGTRLTVL

SEQ ID NO: 244 - protein, 114 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSASPGEEKLFFGSGTQLSVL

SEQ ID NO: 245 - protein, 115 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSASRQVNTEAFFGQGTRLTVV

SEQ ID NO: 246 - protein, 115 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSATLQGDYGYTFGSGTRLTVV

SEQ ID NO: 247 - protein, 116 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSLFGGGSTDTQYFGPGTRLTVL

SEQ ID NO: 248 - protein, 113 aa, Homo sapiens
DADVTQTPRNRITKTGKRIMLECSQTKGHDRMYWYRQDPGLGLRLIYYSFDVKDINKGEISDGYSVSRQAQAKFSLSLESAIPNQTALYFCATSDFQGNYGYTFGSGTRLTVV

SEQ ID NO: 249 - protein, 113 aa, Homo sapiens
DADVTQTPRNRITKTGKRIMLECSQTKGHDRMYWYRQDPGLGLRLIYYSFDVKDINKGEISDGYSVSRQAQAKFSLSLESAIPNQTALYFCATSDSQGLYGYTFGSGTRLTVV

SEQ ID NO: 250 - protein, 112 aa, Homo sapiens
DVKVTQSSRYLVKRTGEKVFLECVQDMDHENMFWYRQDPGLGLRLIYFSYDVKMKEKGDIPEGYSVSREKKERFSLILESASTNQTSMYLCASSRLQDHEQYFGPGTRLTVT

SEQ ID NO: 251 - protein, 112 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSAGQGETQYFGPGTRLLVL

SEQ ID NO: 252 - protein, 112 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSGFLGETQYFGPGTRLLVL

SEQ ID NO: 253 - protein, 112 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSGGQGETQYFGPGTRLLVL

SEQ ID NO: 254 - protein, 114 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSGGQGGTGELFFGEGSRLTVL

SEQ ID NO: 255 - protein, 114 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVAESSNSPLHFGNGTRLTVT

SEQ ID NO: 256 - protein, 113 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVATGWETQYFGPGTRLLVL

SEQ ID NO: 257 - protein, 115 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVDKGGDTDTQYFGPGTRLTVL

SEQ ID NO: 258 - protein, 114 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVEDQSGEKLFFGSGTQLSVL

SEQ ID NO: 259 - protein, 114 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGAGGSGELFFGEGSRLTVL

SEQ ID NO: 260 - protein, 114 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGAGGTGELFFGEGSRLTVL

SEQ ID NO: 261 - protein, 114 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGAVSTDTQYFGPGTRLTVL

SEQ ID NO: 262 - protein, 114 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGGSGANVLTFGAGSRLTVL

SEQ ID NO: 263 - protein, 114 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGLVSTDTQYFGPGTRLTVL

SEQ ID NO: 264 - protein, 114 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGQGGTGELFFGEGSRLTVL

SEQ ID NO: 265 - protein, 114 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGQVSTDTQYFGPGTRLTVL

SEQ ID NO: 266 - protein, 114 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGTVSTDTQYFGPGTRLTVL

SEQ ID NO: 267 - protein, 114 aa, Homo sapiens
SQTIHQWPATLVQPVGSPLSLECTVEGTSNPNLYWYRQAAGRGLQLLFYSVGIGQISSEVPQNLSASRPQDRQFILSSKKLLLSDSGFYLCAWSAQGWDTGELFFGEGSRLTVL

SEQ ID NO: 268 - protein, 114 aa, Homo sapiens
SQTIHQWPATLVQPVGSPLSLECTVEGTSNPNLYWYRQAAGRGLQLLFYSVGIGQISSEVPQNLSASRPQDRQFILSSKKLLLSDSGFYLCAWSPTGWDTGELFFGEGSRLTVL

SEQ ID NO: 269 - protein, 114 aa, Homo sapiens
SQTIHQWPATLVQPVGSPLSLECTVEGTSNPNLYWYRQAAGRGLQLLFYSVGIGQISSEVPQNLSASRPQDRQFILSSKKLLLSDSGFYLCAWSVQGWDTDTQYFGPGTRLTVL

SEQ ID NO: 270 - protein, 114 aa, Homo sapiens
SQTIHQWPATLVQPVGSPLSLECTVEGTSNPNLYWYRQAAGRGLQLLFYSVGIGQISSEVPQNLSASRPQDRQFILSSKKLLLSDSGFYLCAWSVTGWDTGELFFGEGSRLTVL

SEQ ID NO: 271 - protein, 113 aa, Homo sapiens
DTEVTQTPKHLVMGMTNKKSLKCEQHMGHRAMYWYKQKAKKPPELMFVYSYEKLSINESVPSRFSPECPNSSLLNLHLHALQPEDSALYLCASSLSDSDQPQHFGDGTRLSIL

SEQ ID NO: 272 - protein, 112 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSPGPSLGYTFGSGTRLTVV

SEQ ID NO: 273 - protein, 114 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSPRALMNTEAFFGQGTRLTVV

SEQ ID NO: 274 - protein, 115 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSQGLAGREETQYFGPGTRLLVL

SEQ ID NO: 275 - protein, 115 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSQGLAGRQETQYFGPGTRLLVL

SEQ ID NO: 276 - protein, 113 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSQGSGGNEQFFGPGTRLTVL

SEQ ID NO: 277 - protein, 113 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSQRQGGNTIYFGEGSWLTVV

SEQ ID NO: 278 - protein, 112 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSQVAGGEQYFGPGTRLTVT

SEQ ID NO: 279 - protein, 113 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSRGQGATEAFFGQGTRLTVV

SEQ ID NO: 280 - protein, 113 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSRGQGSTEAFFGQGTRLTVV

SEQ ID NO: 281 - protein, 114 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSRLGTSTDTQYFGPGTRLTVL

SEQ ID NO: 282 - protein, 113 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSRTLYQETQYFGPGTRLLVL

SEQ ID NO: 283 - protein, 111 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSFDAETQYFGPGTRLLVL

SEQ ID NO: 284 - protein, 110 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSFEETQYFGPGTRLLVL

SEQ ID NO: 285 - protein, 114 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSFGAGEGDTQYFGPGTRLTVL

SEQ ID NO: 286 - protein, 114 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSFGGGAGDTQYFGPGTRLTVL

SEQ ID NO: 287 - protein, 114 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSFGGPNTGELFFGEGSRLTVL

SEQ ID NO: 288 - protein, 114 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSFGQPSTDTQYFGPGTRLTVL

SEQ ID NO: 289 - protein, 114 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLGAGGQETQYFGPGTRLLVL

SEQ ID NO: 290 - protein, 114 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLGGGAGDTQYFGPGTRLTVL

SEQ ID NO: 291 - protein, 114 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLGGPNTGELFFGEGSRLTVL

SEQ ID NO: 292 - protein, 116 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLGIALSSYNEQFFGPGTRLTVL

SEQ ID NO: 293 - protein, 113 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLGSFSYEQYFGPGTRLTVT

SEQ ID NO: 294 - protein, 116 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLGVALSSYNEQFFGPGTRLTVL

SEQ ID NO: 295 - protein, 114 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLSGPNTDTQYFGPGTRLTVL

SEQ ID NO: 296 - protein, 113 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLVAWDTEAFFGQGTRLTVV

SEQ ID NO: 297 - protein, 112 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSWGMNTEAFFGQGTRLTVV

SEQ ID NO: 298 - protein, 115 aa, Homo sapiens
DAGVTQSPTHLIKTRGQQVTLRCSPISGHKSVSWYQQVLGQGPQFIFQYYEKEERGRGNFPDRFSARQFPNYSSELNVNALLLGDSALYLCASSHRTEYSGNTIYFGEGSWLTVV

SEQ ID NO: 299 - protein, 113 aa, Homo sapiens
DAGVTQSPTHLIKTRGQQVTLRCSPISGHKSVSWYQQVLGQGPQFIFQYYEKEERGRGNFPDRFSARQFPNYSSELNVNALLLGDSALYLCASSLAQGGDTQYFGPGTRLTVL

SEQ ID NO: 300 - protein, 113 aa, Homo sapiens
DAGVTQSPTHLIKTRGQQVTLRCSPISGHKSVSWYQQVLGQGPQFIFQYYEKEERGRGNFPDRFSARQFPNYSSELNVNALLLGDSALYLCASSFGPSNQPQHFGDGTRLSIL

SEQ ID NO: 301 - protein, 112 aa, Homo sapiens
DAGVTQSPTHLIKTRGQQVTLRCSPISGHKSVSWYQQVLGQGPQFIFQYYEKEERGRGNFPDRFSARQFPNYSSELNVNALLLGDSALYLCASSFGVTGELFFGEGSRLTVL

SEQ ID NO: 302 - protein, 112 aa, Homo sapiens
DAGVTQSPTHLIKTRGQQVTLRCSPISGHKSVSWYQQVLGQGPQFIFQYYEKEERGRGNFPDRFSARQFPNYSSELNVNALLLGDSALYLCASSFSVTGELFFGEGSRLTVL

SEQ ID NO: 303 - protein, 112 aa, Homo sapiens
DAGVTQSPTHLIKTRGQQVTLRCSPISGHKSVSWYQQVLGQGPQFIFQYYEKEERGRGNFPDRFSARQFPNYSSELNVNALLLGDSALYLCASSFTNTGELFFGEGSRLTVL

SEQ ID NO: 304 - protein, 112 aa, Homo sapiens
DAGVTQSPTHLIKTRGQQVTLRCSPISGHKSVSWYQQVLGQGPQFIFQYYEKEERGRGNFPDRFSARQFPNYSSELNVNALLLGDSALYLCASSLGRSYGYTFGSGTRLTVV

SEQ ID NO: 305 - protein, 112 aa, Homo sapiens
DAGVTQSPTHLIKTRGQQVTLRCSPISGHKSVSWYQQVLGQGPQFIFQYYEKEERGRGNFPDRFSARQFPNYSSELNVNALLLGDSALYLCASSLKEGYGYTFGSGTRLTVV

SEQ ID NO: 306 - protein, 112 aa, Homo sapiens
DAGVTQSPTHLIKTRGQQVTLRCSPISGHKSVSWYQQVLGQGPQFIFQYYEKEERGRGNFPDRFSARQFPNYSSELNVNALLLGDSALYLCASSLRQLYEQYFGPGTRLTVT

SEQ ID NO: 307 - protein, 112 aa, Homo sapiens
DAGVTQSPTHLIKTRGQQVTLRCSPISGHKSVSWYQQVLGQGPQFIFQYYEKEERGRGNFPDRFSARQFPNYSSELNVNALLLGDSALYLCASSLSGLTEAFFGQGTRLTVV

SEQ ID NO: 308 - protein, 113 aa, Homo sapiens
DAGVTQSPTHLIKTRGQQVTLRCSPISGHKSVSWYQQVLGQGPQFIFQYYEKEERGRGNFPDRFSARQFPNYSSELNVNALLLGDSALYLCASSLVNMNTEAFFGQGTRLTVV

SEQ ID NO: 309 - protein, 112 aa, Homo sapiens
DAGVTQSPTHLIKTRGQQVTLRCSPISGHKSVSWYQQVLGQGPQFIFQYYEKEERGRGNFPDRFSARQFPNYSSELNVNALLLGDSALYLCASSRRQGYGYTFGSGTRLTVV

SEQ ID NO: 310 - protein, 115 aa, Homo sapiens
DAGVTQSPTHLIKTRGQQVTLRCSPISGHKSVSWYQQVLGQGPQFIFQYYEKEERGRGNFPDRFSARQFPNYSSELNVNALLLGDSALYLCASSLRQEYSGNTIYFGEGSWLTVV

SEQ ID NO: 311 - protein, 113 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSTLQGRNGYTFGSGTRLTVV

SEQ ID NO: 312 - protein, 114 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSGRTGRYTEAFFGQGTRLTVV

SEQ ID NO: 313 - protein, 115 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSIRAGGADTQYFGPGTRLTVL

SEQ ID NO: 314 - protein, 115 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSIRTGDGNTQYFGPGTRLTVL

SEQ ID NO: 315 - protein, 115 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSIRTSGSHEQYFGPGTRLTVT

SEQ ID NO: 316 - protein, 116 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLAFLAGEETQYFGPGTRLLVL

SEQ ID NO: 317 - protein, 114 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLAPRTDTQYFGPGTRLTVL

SEQ ID NO: 318 - protein, 115 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRAGGADTQYFGPGTRLTVL

SEQ ID NO: 319 - protein, 115 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRAGGGDTQYFGPGTRLTVL

SEQ ID NO: 320 - protein, 115 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRALDLGEQYFGPGTRLTVT

SEQ ID NO: 321 - protein, 115 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRASGSHEQFFGPGTRLTVL

SEQ ID NO: 322 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRGWETQYFGPGTRLLVL

SEQ ID NO: 323 - protein, 115 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRTSGGHEQFFGPGTRLTVL

SEQ ID NO: 324 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRVGDTQYFGPGTRLTVL

SEQ ID NO: 325 - protein, 115 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRWGGADTQYFGPGTRLTVL

SEQ ID NO: 326 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLVPWETQYFGPGTRLLVL

SEQ ID NO: 327 - protein, 113 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSVRTGDTQYFGPGTRLTVL

SEQ ID NO: 328 - protein, 115 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSPGQGGDNEQFFGPGTRLTVL

SEQ ID NO: 329 - protein, 116 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSPLGGGQDNEQFFGPGTRLTVL

SEQ ID NO: 330 - protein, 113 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSQGQDTEAFFGQGTRLTVV

SEQ ID NO: 331 - protein, 113 aa, Homo sapiens
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSFGSYNEQFFGPGTRLTVL

SEQ ID NO: 332 - protein, 116 aa, Homo sapiens
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSLAAAGGTDTQYFGPGTRLTVL

SEQ ID NO: 333 - protein, 114 aa, Homo sapiens
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSLAGFDSPLHFGNGTRLTVT

SEQ ID NO: 334 - protein, 114 aa, Homo sapiens
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSLAGWDTEAFFGQGTRLTVV

SEQ ID NO: 335 - protein, 118 aa, Homo sapiens
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSLETGTTYSNQPQHFGDGTRLSIL

SEQ ID NO: 336 - protein, 115 aa, Homo sapiens
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSLGTWDTGELFFGEGSRLTVL

SEQ ID NO: 337 - protein, 116 aa, Homo sapiens
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSVLAGAGGDTQYFGPGTRLTVL

SEQ ID NO: 338 - protein, 114 aa, Homo sapiens
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSWLAGTDTQYFGPGTRLTVL

SEQ ID NO: 339 - protein, 113 aa, Homo sapiens
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSYGSYNEQFFGPGTRLTVL

SEQ ID NO: 340 - protein, 114 aa, Homo sapiens
GAGVSQSPRYKVTKRGQDVTLRCDPISSHATLYWYQQALGQGPEFLTYFNYEAQPDKSGLPSDRFSAERPEGSISTLTIQRTEQRDSAMYRCASSFLAGSDTQYFGPGTRLTVL

SEQ ID NO: 341 - protein, 114 aa, Homo sapiens
GAGVSQSPRYKVTKRGQDVTLRCDPISSHATLYWYQQALGQGPEFLTYFNYEAQPDKSGLPSDRFSAERPEGSISTLTIQRTEQRDSAMYRCASSLLAGGDTQYFGPGTRLTVL

SEQ ID NO: 342 - protein, 113 aa, Homo sapiens
GAGVSQSPRYKVAKRGQDVALRCDPISGHVSLFWYQQALGQGPEFLTYFQNEAQLDKSGLPSDRFFAERPEGSVSTLKIQRTQQEDSAVYLCASSFDSNSPLHFGNGTRLTVT

SEQ ID NO: 343 - protein, 113 aa, Homo sapiens
GAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLTQGAGYTFGSGTRLTVV

SEQ ID NO: 344 - protein, 114 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSLGGGAGDTQYFGPGTRLTVL

SEQ ID NO: 345 - protein, 114 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSNILAGEETQYFGPGTRLLVL

SEQ ID NO: 346 - protein, 114 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSVGGGAGDTQYFGPGTRLTVL

SEQ ID NO: 347 - protein, 113 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSVGGVYNEQFFGPGTRLTVL

SEQ ID NO: 348 - protein, 110 aa, Homo sapiens
GQSLEQPSEVTAVEGAIVQINCTYQTSGFYGLSWYQQHDGGAPTFLSYNALDGLEETGRFSSFLSRSDSYGYLLLQELQMKDSASYFCAVTAGSNYQLIWGAGTKLIIKP

SEQ ID NO: 349 - protein, 110 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCAVLTDSWGKLQFGAGTQVVVTP

SEQ ID NO: 350 - protein, 121 aa, Homo sapiens
AQSVTQLGSHVSVSEGALVLLRCNYSSSVPPYLFWYVQYPNQGLQLLLKYTSAATLVKGINGFEAEFKKSETSFHLTKPSAHMSDAAEYFCASLSNFGNEKLTFGNEKLTFGTGTRLTIIP

SEQ ID NO: 351 - protein, 113 aa, Homo sapiens
AQSVTQLGSHVSVSEGALVLLRCNYSSSVPPYLFWYVQYPNQGLQLLLKYTSAATLVKGINGFEAEFKKSETSFHLTKPSAHMSDAAEYFCAVSEWAGNQFYFGTGTSLTVIP

SEQ ID NO: 352 - protein, 114 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRLTLGTDTQYFGPGTRLTVL

SEQ ID NO: 353 - protein, 115 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRPRGAPSYEQYFGPGTRLTVT

SEQ ID NO: 354 - protein, 113 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSWTSWDTQYFGPGTRLTVL

SEQ ID NO: 355 - protein, 112 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSPGPSLGYTFGSGTRLTVV

SEQ ID NO: 356 - protein, 114 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSPRALMNTEAFFGQGTRLTVV

SEQ ID NO: 357 - protein, 115 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQGLAGREETQYFGPGTRLLVL

SEQ ID NO: 358 - protein, 115 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQGLAGRQETQYFGPGTRLLVL

SEQ ID NO: 359 - protein, 113 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQGSGGNEQFFGPGTRLTVL

SEQ ID NO: 360 - protein, 113 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQRQGGNTIYFGEGSWLTVV

SEQ ID NO: 361 - protein, 112 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQVAGGEQYFGPGTRLTVT

SEQ ID NO: 362 - protein, 113 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSRGQGATEAFFGQGTRLTVV

SEQ ID NO: 363 - protein, 113 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSRGQGSTEAFFGQGTRLTVV

SEQ ID NO: 364 - protein, 114 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSRLGTSTDTQYFGPGTRLTVL

SEQ ID NO: 365 - protein, 113 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSRTLYQETQYFGPGTRLLVL

SEQ ID NO: 366 - protein, 113 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASSFGPSNQPQHFGDGTRLSIL

SEQ ID NO: 367 - protein, 112 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASSFGVTGELFFGEGSRLTVL

SEQ ID NO: 368 - protein, 112 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASSFSVTGELFFGEGSRLTVL

SEQ ID NO: 369 - protein, 112 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASSFTNTGELFFGEGSRLTVL

SEQ ID NO: 370 - protein, 112 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASSLGRSYGYTFGSGTRLTVV

SEQ ID NO: 371 - protein, 112 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASSLKEGYGYTFGSGTRLTVV

SEQ ID NO: 372 - protein, 112 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASSLRQLYEQYFGPGTRLTVT

SEQ ID NO: 373 - protein, 112 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASSLSGLTEAFFGQGTRLTVV

SEQ ID NO: 374 - protein, 113 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASSLVNMNTEAFFGQGTRLTVV

SEQ ID NO: 375 - protein, 112 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASSRRQGYGYTFGSGTRLTVV

SEQ ID NO: 376 - protein, 115 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASSLRQEYSGNTIYFGEGSWLTVV

SEQ ID NO: 377 - protein, 113 aa, Homo sapiens; identical to SEQ ID NO: 311

SEQ ID NO: 378 - protein, 113 aa, Homo sapiens
  MISC_FEATURE (96)..(96): Xaa is Phe or Ser
  MISC_FEATURE (99)..(99): Xaa is Leu or Asn
DADVTQTPRNRITKTGKRIMLECSQTKGHDRMYWYRQDPGLGLRLIYYSFDVKDINKGEISDGYSVSRQAQAKFSLSLESAIPNQTALYFCATSDXQGXYGYTFGSGTRLTVV

SEQ ID NO: 379 - protein, 114 aa, Homo sapiens
  MISC_FEATURE (95)..(95): Xaa is any naturally occurring amino acid
  MISC_FEATURE (97)..(97): Xaa is Ala or Gln
  MISC_FEATURE (100)..(100): Xaa is Ser or Thr
SAVISQKPSRDICQRGTSLTIQCQVDSQVTMMFWYRQQPGQSLTLIATANQGSEATYESGFVIDKFPISRPNLTFSTLTVSNMSPEDSSIYLCSXGXGGXGELFFGEGSRLTVL

SEQ ID NO: 380 - protein, 114 aa, Homo sapiens
  misc_feature (97)..(97): Xaa can be any naturally occurring amino acid
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGXVSTDTQYFGPGTRLTVL

SEQ ID NO: 381 - protein, 112 aa, Homo sapiens
  MISC_FEATURE (95)..(95): Xaa is Ala or Gly
  MISC_FEATURE (96)..(96): Xaa is Phe or Gly
  MISC_FEATURE (97)..(97): Xaa is Leu or Gln
SAVISQKPSRDICQRGTSLTIQCQVDSQVTMMFWYRQQPGQSLTLIATANQGSEATYESGFVIDKFPISRPNLTFSTLTVSNMSPEDSSIYLCSXXXGETQYFGPGTRLLVL

SEQ ID NO: 382 - protein, 114 aa, Homo sapiens
  MISC_FEATURE (95)..(95): Xaa is any naturally occurring amino acid
  MISC_FEATURE (96)..(96): Xaa is Gln or Thr
SQTIHQWPATLVQPVGSPLSLECTVEGTSNPNLYWYRQAAGRGLQLLFYSVGIGQISSEVPQNLSASRPQDRQFILSSKKLLLSDSGFYLCAWSXXGWDTGELFFGEGSRLTVL

SEQ ID NO: 383 - protein, 113 aa, Homo sapiens
  MISC_FEATURE (99)..(99): Xaa is Ala or Ser
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSRGQGXTEAFFGQGTRLTVV

SEQ ID NO: 384 - protein, 115 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSQGLAGRZETQYFGPGTRLLVL

SEQ ID NO: 385 - protein, 116 aa, Homo sapiens
  MISC_FEATURE (97)..(97): Xaa is Ile or Val
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLGXALSSYNEQFFGPGTRLTVL

SEQ ID NO: 386 - protein, 114 aa, Homo sapiens
  MISC_FEATURE (95)..(95): Xaa is Phe or Leu
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSXGGPNTGELFFGEGSRLTVL

SEQ ID NO: 387 - protein, 114 aa, Homo sapiens
  MISC_FEATURE (95)..(95): Xaa is Phe or Leu
  MISC_FEATURE (96)..(96): Xaa is Ser or Gly
  MISC_FEATURE (97)..(97): Xaa is any naturally occurring amino acid
  MISC_FEATURE (98)..(98): Xaa is Pro or Gly
  MISC_FEATURE (99)..(99): Xaa is any naturally occurring amino acid
  MISC_FEATURE (100)..(100): Xaa is Thr or Gly
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSXXXXXXDTQYFGPGTRLTVL

SEQ ID NO: 388 - protein, 111 aa, Homo sapiens
  MISC_FEATURE (97)..(97): Xaa is Ala or Gly
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSFDXETQYFGPGTRLLVL

SEQ ID NO: 389 - protein, 112 aa, Homo sapiens
  MISC_FEATURE (95)..(95): Xaa is Leu or Arg
  MISC_FEATURE (96)..(97): Xaa is any naturally occurring amino acid
  MISC_FEATURE (98)..(98): Xaa is Ser or Gly
DAGVTQSPTHLIKTRGQQVTLRCSPISGHKSVSWYQQVLGQGPQFIFQYYEKEERGRGNFPDRFSARQFPNYSSELNVNALLLGDSALYLCASSXXXXYGYTFGSGTRLTVV

SEQ ID NO: 390 - protein, 112 aa, Homo sapiens
  MISC_FEATURE (96)..(96): Xaa is any naturally occurring amino acid
  MISC_FEATURE (97)..(97): Xaa is Val or Asn
DAGVTQSPTHLIKTRGQQVTLRCSPISGHKSVSWYQQVLGQGPQFIFQYYEKEERGRGNFPDRFSARQFPNYSSELNVNALLLGDSALYLCASSFXXTGELFFGEGSRLTVL

SEQ ID NO: 391 - protein, 115 aa, Homo sapiens
  MISC_FEATURE (98)..(98): Xaa is Ala or Thr
  MISC_FEATURE (101)..(101): Xaa is Gly or Ser
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRXSGXHEQFFGPGTRLTVL

SEQ ID NO: 392 - protein, 115 aa, Homo sapiens
  MISC_FEATURE (96)..(96): Xaa is Ile or Leu
  MISC_FEATURE (98)..(98): Xaa is any naturally occurring amino acid
  MISC_FEATURE (100)..(100): Xaa is Gly or Asp
  MISC_FEATURE (101)..(101): Xaa is Ala or Gly
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSXRXGXXBTQYFGPGTRLTVL

SEQ ID NO: 393 - protein, 113 aa, Homo sapiens
  MISC_FEATURE (96)..(96): Xaa is Leu or Val
  MISC_FEATURE (98)..(98): Xaa is Thr or Val
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSXRXGDTQYFGPGTRLTVL

SEQ ID NO: 394 - protein, 113 aa, Homo sapiens
  MISC_FEATURE (97)..(97): Xaa is Arg or Val
  MISC_FEATURE (98)..(98): Xaa is Pro or Gly
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLXXWETQYFGPGTRLLVL

SEQ ID NO: 395 - protein, 113 aa, Homo sapiens
  MISC_FEATURE (96)..(96): Xaa is Phe or Tyr
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSXGSYNEQFFGPGTRLTVL

SEQ ID NO: 396 - protein, 116 aa, Homo sapiens
  MISC_FEATURE (96)..(96): Xaa is Leu or Val
  MISC_FEATURE (97)..(97): Xaa is Leu or Ala
  MISC_FEATURE (98)..(98): Xaa is Ala or Ser
  MISC_FEATURE (99)..(100): Xaa is Ala or Gly
  MISC_FEATURE (102)..(102): Xaa is Thr or Gly
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSXXXXXGXDTQYFGPGTRLTVL

SEQ ID NO: 397 - protein, 114 aa, Homo sapiens
  misc_feature (96)..(96): Xaa can be any naturally occurring amino acid
  misc_feature (100)..(100): Xaa can be any naturally occurring amino acid
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSXLAGXDTQYFGPGTRLTVL

SEQ ID NO: 398 - protein, 114 aa, Homo sapiens
  MISC_FEATURE (95)..(95): Xaa is Leu or Val
DSGVTQTPKHLITATGQRVTLRCSPRSGDLSVYWYQQSLDQGLQFLIQYYNGEERAKGNILERFSAQQFPDLHSELNLSSLELGDSALYFCASSXGGGAGDTQYFGPGTRLTVL

SEQ ID NO: 399 - protein, 110 aa, Homo sapiens
  MISC_FEATURE (93)..(93): Xaa is Ser or Thr
GQNIDQPTEMTATEGAIVQINCTYQTSGFNGLFWYQQHAGEAPTFLSYNVLDGLEEKGRFSSFLSRSKGYSYLLLKELQMKDSASYLCAVTSXNTGKLIFGQGTTLQVKP

SEQ ID NO: 400 - protein, 113 aa, Homo sapiens
  misc_feature (94)..(94): Xaa can be any naturally occurring amino acid
GQNIDQPTEMTATEGAIVQINCTYQTSGFNGLFWYQQHAGEAPTFLSYNVLDGLEEKGRFSSFLSRSKGYSYLLLKELQMKDSASYLCAVRAVXSGGYNKLIFGAGTRLAVHP

SEQ ID NO: 401 - protein, 111 aa, Homo sapiens
  MISC_FEATURE (92)..(92): Xaa is any naturally occurring amino acid
  MISC_FEATURE (93)..(93): Xaa is Ala or Tyr
RKEVEQDPGPFNVPEGATVAFNCTYSNSASQSFFWYRQDCRKEPKLLMSVYSSGNEDGRFTAQLNRASQYISLLIRDSKLSDSATYLCVVNXXSSASKIIFGSGTRLSIRP

SEQ ID NO: 402 - protein, 110 aa, Homo sapiens
  MISC_FEATURE (92)..(92): Xaa is Pro or Ser
QKEVEQNSGPLSVPEGAIASLNCTYSDRGSQSFFWYRQYSGKSPELIMFIYSNGDKEDGRFTAQLNKASQYVSLLIRDSQPSDSATYLCAVXNRDDKIIFGKGTRLHILP

SEQ ID NO: 403 - protein, 111 aa, Homo sapiens
  misc_feature (92)..(92): Xaa can be any naturally occurring amino acid
QKEVEQDPGPLSVPEGAIVSLNCTYSNSAFQYFMWYRQYSRKGPELLMYTYSSGNKEDGRFTAQVDKSSKYISLFIRDSQPSDSATYLCAMXZAAGNKLTFGGGTRVLVKP

SEQ ID NO: 404 - protein, 110 aa, Homo sapiens
  MISC_FEATURE (92)..(92): Xaa is Lys or Arg
QKEVEQDPGPLSVPEGAIVSLNCTYSNSAFQYFMWYRQYSRKGPELLMYTYSSGNKEDGRFTAQVDKSSKYISLFIRDSQPSDSATYLCAMXDYGQNFVFGPGTRLSVLP

SEQ ID NO: 405 - protein, 111 aa, Homo sapiens
  MISC_FEATURE (93)..(93): Xaa is Ala or Glu
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSXGTGNQFYFGTGTSLTVIP

SEQ ID NO: 406 - protein, 114 aa, Homo sapiens
  MISC_FEATURE (93)..(93): Xaa is Asp or Glu
  MISC_FEATURE (94)..(94): Xaa is Ile or Leu
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSXXPGGSNYKLTFGKGTLLTVNP

SEQ ID NO: 407 - protein, 111 aa, Homo sapiens
  MISC_FEATURE (92)..(92): Xaa is Asn or Thr
QKEVEQDPGPLSVPEGAIVSLNCTYSNSAFQYFMWYRQYSRKGPELLMYTYSSGNKEDGRFTAQVDKSSKYISLFIRDSQPSDSATYLCAMXDYGNNRLAFGKGNQVVVIP

SEQ ID NO: 408 - protein, 115 aa, Homo sapiens
  MISC_FEATURE (93)..(93): Xaa is Gly or Thr
GESVGLHLPTLSVQEGDNSIINCAYSNSASDYFIWYKQESGKGPQFIIDIRSNMDKRQGQRVTVLLNKTVKHLSLQIAATQPGDSAVYFCAEXBAGGTSYGKLTFGQGTILTVHP

SEQ ID NO: 409 - protein, 116 aa, Homo sapiens
  MISC_FEATURE (98)..(98): Xaa is Gly or Ser
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEGXNAGNMLTFGGGTRLMVKP

SEQ ID NO: 410 - protein, 115 aa, Homo sapiens
  MISC_FEATURE (96)..(96): Xaa is Gly or Glu
  MISC_FEATURE (97)..(97): Xaa is Gly or Ala
AQKVTQAQTEISVVEKEDVTLDCVYETRDTTYYLFWYKQPPSGELVFLIRRNSFDEQNEISGRYSWNFQKSTSSFNFTITASQVVDSAVYFCALSXXGANSKLTFGKGITLSVRP

SEQ ID NO: 411 - protein, 109 aa, Homo sapiens
  MISC_FEATURE (91)..(91): Xaa is Leu or Arg
GIQVEQSPPDLILQEGANSTLRCNFSDSVNNLQWFHQNPWGQLINLFYIPSGTKQNGRLSATTVATERYSLLYISSSQTTDSGVYFCAVEXQGAQKLVFGQGTRLTINP

SEQ ID NO: 412 - protein, 108 aa, Homo sapiens
  MISC_FEATURE (90)..(90): Xaa is any naturally occurring amino acid
  MISC_FEATURE (91)..(91): Xaa is Phe or Tyr
DAKTTQPPSMDCAEGRAANLPCNHSTISGNEYVYWYRQIHSQGPQYIIHGLKNNETNEMASLIITEDRKSSTLILPHATLRDTAVYYCIXXNDYKLSFGAGTTVTVRA

SEQ ID NO: 413 - protein, 107 aa, Homo sapiens
  misc_feature (91)..(91): Xaa can be any naturally occurring amino acid
DAKTTQPPSMDCAEGRAANLPCNHSTISGNEYVYWYRQIHSQGPQYIIHGLKNNETNEMASLIITEDRKSSTLILPHATLRDTAVYYCIVXNARLMFGDGTQLVVKP

SEQ ID NO: 414 - protein, 111 aa, Homo sapiens
  MISC_FEATURE (90)..(90): Xaa is Gly or Pro
DAKTTQPPSMDCAEGRAANLPCNHSTISGNEYVYWYRQIHSQGPQYIIHGLKNNETNEMASLIITEDRKSSTLILPHATLRDTAVYYCIXNYGGSQGNLIFGKGTKLSVKP

SEQ ID NO: 415 - protein, 110 aa, Homo sapiens
  MISC_FEATURE (91)..(91): Xaa is Phe or Tyr
DAKTTQPPSMDCAEGRAANLPCNHSTISGNEYVYWYRQIHSQGPQYIIHGLKNNETNEMASLIITEDRKSSTLILPHATLRDTAVYYCIVXGGSQGNLIFGKGTKLSVKP

SEQ ID NO: 416 - protein, 108 aa, Homo sapiens
  MISC_FEATURE (91)..(91): Xaa is Ala or Gly
GQQLNQSPQSMFIQEGEDVSMNCTSSSIFNTWLWYKQEPGEGPVLLIALYKAGELTSNGRLTAQFGITRKDSFLNISASIPSDVGIYFCAXNDYKLSFGAGTTVTVRA

SEQ ID NO: 417 - protein, 112 aa, Homo sapiens
  MISC_FEATURE (92)..(92): Xaa is Asn or Gln
  MISC_FEATURE (93)..(93): Xaa is Leu or Tyr
GQQLNQSPQSMFIQEGEDVSMNCTSSSIFNTWLWYKQEPGEGPVLLIALYKAGELTSNGRLTAQFGITRKDSFLNISASIPSDVGIYFCAGXXGGATNKLIFGTGTLLAVQP

SEQ ID NO: 418 - protein, 109 aa, Homo sapiens
  MISC_FEATURE (92)..(92): Xaa is Phe or His
GQQLNQSPQSMFIQEGEDVSMNCTSSSIFNTWLWYKQEPGEGPVLLIALYKAGELTSNGRLTAQFGITRKDSFLNISASIPSDVGIYFCAGXNTDKLIFGTGTRLQVFP

SEQ ID NO: 419 - protein, 113 aa, Homo sapiens
  MISC_FEATURE (93)..(93): Xaa is Pro or Val
GQQLNQSPQSMFIQEGEDVSMNCTSSSIFNTWLWYKQEPGEGPVLLIALYKAGELTSNGRLTAQFGITRKDSFLNISASIPSDVGIYFCAGQXGSSNTGKLIFGQGTTLQVKP

SEQ ID NO: 420 - protein, 111 aa, Homo sapiens
  MISC_FEATURE (92)..(92): Xaa is Asp or Gly
  MISC_FEATURE (93)..(93): Xaa is any naturally occurring amino acid
LAKTTQPISMDSYEGQEVNITCSHNNIATNDYITWYQQFPSQGPRFIIQGYKTKVTNEVASLFIPADRKSSTLSLPRVSLSDTAVYYCLVGXXGGYNKLIFGAGTRLAVHP

SEQ ID NO: 421 - protein, 111 aa, Homo sapiens
  MISC_FEATURE (93)..(93): Xaa is Asp or Pro
LAKTTQPISMDSYEGQEVNITCSHNNIATNDYITWYQQFPSQGPRFIIQGYKTKVTNEVASLFIPADRKSSTLSLPRVSLSDTAVYYCLVGDXTGFQKLVFGTGTRLLVSP

SEQ ID NO: 422 - protein, 109 aa, Homo sapiens
  misc_feature (91)..(91): Xaa can be any naturally occurring amino acid
KNEVEQSPQNLTAQEGEFITINCSYSVGISALHWLQQHPGGGIVSLFMLSSGKKKHGRLIATINIQEKHSSLHITASHPRDSAVYICAVEXGSNYQLIWGAGTKLIIKP

SEQ ID NO: 423 - protein, 116 aa, Homo sapiens
  MISC_FEATURE (94)..(94): Xaa is Ala or Gly
  MISC_FEATURE (95)..(95): Xaa is any naturally occurring amino acid
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVXXDRGSTLGRLYFGRGTQLTVWP

SEQ ID NO: 424 - protein, 114 aa, Homo sapiens
  MISC_FEATURE (95)..(95): Xaa is any naturally occurring amino acid
  MISC_FEATURE (96)..(96): Xaa is Ala or Ser
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGXXEYGNKLVFGAGTILRVKS

SEQ ID NO: 425 - protein, 112 aa, Homo sapiens
  MISC_FEATURE (93)..(93): Xaa is Ala or Ser
  MISC_FEATURE (95)..(95): Xaa is Tyr or Gly
GNSVTQMEGPVTLSEEAFLTINCTYTATGYPSLFWYVQYPGEGLQLLLKATKADDKGSNKGFEATYRKETTSFHLEKGSVQVSDSAVYFCALXEXNFNKFYFGSGTKLNVKP

SEQ ID NO: 426 - protein, 114 aa, Homo sapiens
  MISC_FEATURE (95)..(95): Xaa is Pro or Gln
GNSVTQMEGPVTLSEEAFLTINCTYTATGYPSLFWYVQYPGEGLQLLLKATKADDKGSNKGFEATYRKETTSFHLEKGSVQVSDSAVYFCALSDXTGTASKLTFGTGTRLQVTL

SEQ ID NO: 427 - protein, 114 aa, Homo sapiens
  MISC_FEATURE (96)..(96): Xaa is Leu or Pro
NAGVMQNPRHLVRRRGQEARLRCSPMKGHSHVYWYRQLPEEGLKFMVYLQKENIIDESGMPKERFSAEFPKEGPSILRIQQVVRGDSAAYFCASSXAGWDTEAFFGQGTRLTVV

SEQ ID NO: 428 - protein, 113 aa, Homo sapiens
  MISC_FEATURE (95)..(95): Xaa is Ile or Thr
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSXGTSGETQYFGPGTRLLVL

SEQ ID NO: 429 - protein, 113 aa, Homo sapiens
  MISC_FEATURE (99)..(99): Xaa is Ala or Ser
ETGVTQTPRHLVMGMTNKKSLKCEQHLGHNAMYWYKQSAKKPLELMFVYSLEERVENNSVPSRFSPECPNSSHLFLHLHTLQPEDSALYLCASSRGQGXTEAFFGQGTRLTVV

SEQ ID NO: 430 - protein, 115 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQGLAGRZETQYFGPGTRLLVL

SEQ ID NO: 431 - protein, 112 aa, Homo sapiens
  MISC_FEATURE (95)..(95): Xaa is Leu or Arg
  MISC_FEATURE (96)..(97): Xaa is any naturally occurring amino acid
  MISC_FEATURE (98)..(98): Xaa is Ser or Gly
DAGVTQSPTHLIKTRGQQVTLRCSPKSGHDTVSWYQQALGQGPQFIFQYYEEEERQRGNFPDRFSGHQFPNYSSELNVNALLLGDSALYLCASSXXXXYGYTFGSGTRLTVV

SEQ ID NO: 432 - protein, 112 aa, Homo sapiens
  MISC_FEATURE (96)..(96): Xaa is any naturally occurring amino acid
  MISC_FEATURE (97)..(97): Xaa is Val or Asn
DAGVTQSPTHLIKTRGQQVTLRCSPKSGHDTVSWYQQALGQGPQFIFQYYEEEERQRGNFPDRFSGHQFPNYSSELNVNALLLGDSALYLCASSFXXTGELFFGEGSRLTVL

(19) United States
(12) Patent Application Publication
(10) Pub. No.: US 2021/0010077 A1
(43) Pub. Date: Jan. 14, 2021

(54) METHOD OF DIAGNOSING CELIAC DISEASE

(71) Applicants: OSLO UNIVERSITETSSYKEHUS HF, Aslo (NO); UNIVERSITETET I OSLO, Oslo (NO)

(72) Inventors: Ludvig M. SOLLID, Bekkestua (NO); Shuo-Wang QIAO, Nesøya (NO); Ralf Stefan NEUMANN, Oslo (NO); Geir Kjetil SANDVE, Oslo (NO); Louise Fremgaard RISNES, Oslo (NO); Asbjørn CHRISTOPHERSEN, Oslo (NO); Shiva DAHAL-KOIRALA, Lørenskog (NO); Knut E. A. LUNDIN, Oslo (NO)

(21) Appl. No.: 16/981,431

(22) PCT Filed: Mar. 25, 2019
(86) PCT No.: PCT/EP2019/057428

§ 371 Date: Sep. 16, 2020

(30) Foreign Application Priority Data
Mar. 23, 2018 (GB) 1804724.1

(51) Int. Cl. C12Q 1/6869; C12Q 1/686; C12Q 1/6876; C12N 15/10; C12N 5/0783; G01N 33/50

(52) U.S. Cl. CPC C12Q 1/6869; C12Q 1/686; C12Q 1/6876; C12N 15/1096; G01N 2800/52; C12N 5/0636; G01N 33/505; G01N 2800/06; C12N 15/1089

(57) ABSTRACT

The present invention relates to a method for diagnosing celiac disease in a subject, or monitoring a subjects response to treatment for celiac disease. The method comprises analysing the subjects TCR repertoire for the presence of gluten-specific TCR sequences, determining a normalised score for the frequency of the gluten-specific TCR sequences in the subjects TCR repertoire and comparing the normalised score to a pre-determined disease threshold.

### FIELD OF THE INVENTION

The present disclosure pertains generally to methods for diagnosis of celiac disease, and provides a non-invasive diagnostic test.

### BACKGROUND

Celiac disease is an autoimmune disorder in which an aberrant immune response to gluten (a composite of storage proteins found in cereal plants, particularly wheat and barley) results in damage to various organs. Primarily affected is the small intestine, which may become inflamed and undergo a number of pathological changes. Sufferers of celiac disease may have abdominal pain and cramping, while the pathological changes to the small intestine negatively impacts nutrient absorption, which can result in weight loss and anaemia. Celiac disease sufferers may also be at higher risk of cancer in the small intestine. The only current treatment for celiac disease is adoption of a gluten-free diet. The cause of celiac disease is not fully understood, though it is known to have a genetic component: the majority of celiac disease patients (˜90%) carry the HLA-DQ allele HLA-DQ2.5, while the remainder of cases occur in individuals carrying the HLA-DQ2.2 or HLA-DQ8 alleles.

The existing gold standard for celiac disease (CD) diagnosis of adults requires examination of intestinal biopsies taken during endoscopic procedure of the upper gastro-intestinal tract. This procedure must be performed by an endoscopist, and requires specialist equipment and infrastructure that is usually only available in hospitals and large clinics. Biopsy samples are examined and categorised by the Marsh Classification, according to which celiac disease is diagnosed based on the pathology of the intestinal mucosa. Prior to biopsy an initial blood test may also be carried out; elevated serum levels of antibodies against transglutaminase 2 (TG2) and/or deamidated gliadin peptide (DGP) are indicative of celiac disease.

Upon adoption of a gluten-free diet, the currently-used diagnostic parameters (both antibody markers in serum and the pathology of the intestinal mucosa) normalise and render the existing diagnostic tools largely ineffective. With the increasing incidence of gluten-free diet adoption by individuals without a celiac disease diagnosis, or who have self-diagnosed as gluten-intolerant, the demand for diagnostic tests that are effective in subjects adhering to a gluten-free diet is increasing.

WO 2014/179202 mentions a method of diagnosing celiac disease by detecting activated, gut-bound CD8+αβ T lymphocytes and γδ T lymphocytes in the peripheral blood of a subject who has consumed gluten for one to three days. The method requires that the individual adheres to a gluten-free diet prior to the challenge, and voluntary gluten ingestion by the subject, which may be undesirable for an individual with a gluten intolerance.

Ritter, J. et al., (Gut 67(4): 644-653, 2018), disclosed high-throughput sequencing for establishing the T-cell repertoire in CD and refractory CD (RCD), particularly Type II RCD, to unravel the role of distinct T-cell clonotypes in RCD pathogenesis. It was found that the dominant T-cell clones of patients with Type II RCD are private, i.e. unique to each patient.

Yohannes, D. et al., (Scientific Reports 7:17977, 2017), performed deep sequencing of blood and gut T-cell receptor (TCR) β-chains to identify gluten-induced immune signatures in sufferers of celiac disease. The authors reported increased overlap of individual TCR repertoires during gluten exposure, and identified major immunological signatures associated with gluten exposure in celiac disease sufferers.

Sarna, V. K. et al. (Gastroenterology 154: 886-896, 2018) disclose the use of HLA-DQ-gluten tetramers to identify gluten-specific T-cells. The tetramers comprise recombinant HLA-DQ2.5 molecules presenting commonly-recognised gluten epitopes multimerised on fluorescent-labelled streptavidin, and are used to identify and isolate gluten-binding T-cells. The authors disclose that the identification of gluten-binding T-cells in a subject may be indicative of celiac disease.

### SUMMARY

The present disclosure provides a method for diagnosing celiac disease. The method does not require the performance of biopsies or upfront gluten ingestion by the subject, and is therefore advantageous over the current gold-standard diagnostic tests. Since the method may be performed on an individual consuming a gluten-free diet, the accuracy of the test is not dependent on compliance of the subject with a particular dietary regime, and the absence of a requirement for a biopsy means the method is not invasive; sample collection can be carried out by a nurse or general practitioner, and the likelihood of complications is significantly reduced.

It has been found that analysis of the number of T-cells in a sample expressing TCR chains as specified in Tables 1, 2 and 3 indicates whether a patient suffers from celiac disease.

Accordingly, the method is quick, convenient and reliable. Arriving at this method was not trivial. The method was conceived based on several important findings described herein, including that identical gluten-specific clonotypes are found in peripheral blood and gut mucosa. Furthermore, it was observed that the frequency of gluten-specific CD4+ T-cells decreases upon adoption of a gluten-free diet (GFD), but that the same clonotypes are found in multiple samples taken weeks to years apart. It was also found that gluten-specific memory T-cells expand and dominate on oral gluten challenge and that the dominance of memory clonotypes 28 days after reintroduction of gluten was unchanged. In fact, a similar fraction of clonotypes is observed 6 months and 27 years apart. It was also found that at least 10% of gluten-specific T-cells use public TCR sequences, of which some can be utilised for diagnosing celiac disease.

Some gluten-specific TCR sequences have already been detected in patients with celiac disease (see Table 1). However, numerous hitherto unknown public TCR sequences connected to celiac disease, listed in Table 2, are provided herein. Furthermore, a group of consensus TCR sequences, listed in Table 3, can be generalised from the sequences in Table 2. Together with the TCR sequences in Table 1, these TCR sequences can be used for diagnosing celiac disease based on quantifying their relative abundance in peripheral blood mononuclear cells, in particular their relative abundance in effector memory CD4+ T-cells. Because some of these sequences also appear in healthy controls, the method disclosed herein offers greater specificity of diagnosis than does a purely binary sequence detection method. Accordingly, the sequences specified in Table 1 and Table 2 together make up a powerful reference tool, allowing non-invasive diagnosis of celiac disease. The sequences specified in Table 3 are a useful addition to this tool. In addition to diagnosing celiac disease, the method is equally useful for ruling out a diagnosis of celiac disease in a patient with symptoms of gluten intolerance. Although it is preferred that the diagnostic test for celiac disease disclosed herein is performed non-invasively on a blood sample, the disclosed method can equally be performed on a sample obtained by biopsy.

In a first aspect, provided herein is an in vitro method for diagnosing celiac disease in a human subject or monitoring the response of a human subject to treatment therefor, said method comprising the steps:
a) isolating nucleic acids from a sample obtained from the subject, wherein said sample comprises T-cells;
b) sequencing nucleotide sequences which encode TCRα chains and nucleotide sequences which encode TCRβ chains to provide a TCR dataset;
c) assigning a score to the TCR dataset, wherein said score is determined by the abundance in the dataset of nucleotide sequences which encode at least two TCRα or TCRβ amino acid sequences, wherein said at least two TCRα or TCRβ amino acid sequences comprise:
   (i) at least one TCRα or TCRβ amino acid sequence selected from SEQ ID NOs: 1 to 50; and
   (ii) at least one TCRα or TCRβ amino acid sequence selected from SEQ ID NOs: 51 to 432;
d) normalising said score to provide a normalised score representative of:
   (i) the frequency of the nucleotide sequences in the TCR dataset; or
   (ii) the frequency of T-cells expressing the nucleotide sequences in the sample; and
e) comparing said normalised score to a defined threshold, wherein the subject is diagnosed with celiac disease if said normalised score is equal to or higher than the defined threshold, or the response to treatment is determined by comparison to the defined threshold.

In a related aspect, also provided herein is a method for diagnosing celiac disease in a human subject or monitoring the response of a human subject to treatment therefor, said method comprising the steps:
a) obtaining a sample comprising T-cells from the subject;
b) isolating nucleic acids from the sample;
c) sequencing nucleotide sequences which encode TCRα chains and nucleotide sequences which encode TCRβ chains to provide a TCR dataset;
d) assigning a score to the TCR dataset, wherein said score is determined by the abundance in the dataset of nucleotide sequences which encode at least two TCRα or TCRβ amino acid sequences, wherein said at least two TCRα or TCRβ amino acid sequences comprise:
   (i) at least one TCRα or TCRβ amino acid sequence selected from SEQ ID NOs: 1 to 50; and
   (ii) at least one TCRα or TCRβ amino acid sequence selected from SEQ ID NOs: 51 to 432;
e) normalising said score to provide a normalised score representative of:
   (i) the frequency of the nucleotide sequences in the TCR dataset; or
   (ii) the frequency of T-cells expressing the nucleotide sequences in the sample; and
f) comparing said normalised score to a defined threshold, wherein the subject is diagnosed with celiac disease if said normalised score is equal to or higher than the defined threshold, or the response to treatment is determined by comparison to the defined threshold.

In another aspect, provided herein is a method for diagnosing and treating celiac disease in a human subject, said method comprising the steps:
a) isolating nucleic acids from a sample obtained from the subject, wherein said sample comprises T-cells;
b) sequencing nucleotide sequences which encode TCRα chains and nucleotide sequences which encode TCRβ chains to provide a TCR dataset;
c) assigning a score to the TCR dataset, wherein said score is determined by the abundance in the dataset of nucleotide sequences which encode at least two TCRα or TCRβ amino acid sequences, wherein said at least two TCRα or TCRβ amino acid sequences comprise:
   (i) at least one TCRα or TCRβ amino acid sequence selected from SEQ ID NOs: 1 to 50; and
   (ii) at least one TCRα or TCRβ amino acid sequence selected from SEQ ID NOs: 51 to 432;
d) normalising said score to provide a normalised score representative of:
   (i) the frequency of the nucleotide sequences in the TCR dataset; or
   (ii) the frequency of T-cells expressing the nucleotide sequences in the sample;
e) comparing said normalised score to a defined threshold, wherein the subject is diagnosed with celiac disease if said normalised score is equal to or higher than the defined threshold; and
f) if the subject is diagnosed with celiac disease, administering treatment for celiac disease to the subject.

In a related aspect, provided herein is a method for diagnosing and treating celiac disease in a human subject, said method comprising the steps:
a) obtaining a sample comprising T-cells from the subject;
b) isolating nucleic acids from the sample;
c) sequencing nucleotide sequences which encode TCRα chains and nucleotide sequences which encode TCRβ chains to provide a TCR dataset;
d) assigning a score to the TCR dataset, wherein said score is determined by the abundance in the dataset of nucleotide sequences which encode at least two TCRα or TCRβ amino acid sequences, wherein said at least two TCRα or TCRβ amino acid sequences comprise:
   (i) at least one TCRα or TCRβ amino acid sequence selected from SEQ ID NOs: 1 to 50; and
   (ii) at least one TCRα or TCRβ amino acid sequence selected from SEQ ID NOs: 51 to 432;
e) normalising said score to provide a normalised score representative of:
   (i) the frequency of the nucleotide sequences in the TCR dataset; or
   (ii) the frequency of T-cells expressing the nucleotide sequences in the sample;
f) comparing said normalised score to a defined threshold, wherein the subject is diagnosed with celiac disease if said normalised score is equal to or higher than the defined threshold; and
g) if the subject is diagnosed with celiac disease, administering treatment for celiac disease to the subject.

In another aspect, provided herein is a method for detecting TCR sequences in cells in a sample, said method comprising the steps:
a) isolating nucleic acids from a sample obtained from a human subject, wherein the sample comprises T-cells;
b) sequencing nucleotide sequences which encode TCRα chains and nucleotide sequences which encode TCRβ chains to provide a TCR dataset;
c) assigning a score to the TCR dataset, wherein said score is determined by the abundance in the dataset of nucleotide sequences which encode at least two gluten-specific TCRα or TCRβ amino acid sequences, wherein said at least two gluten-specific TCRα or TCRβ amino acid sequences comprise:
   (i) at least one TCRα or TCRβ amino acid sequence selected from SEQ ID NOs: 1 to 50; and
   (ii) at least one TCRα or TCRβ amino acid sequence selected from SEQ ID NOs: 51 to 432;
d) normalising said score to provide a normalised score representative of:
   (i) the frequency of the nucleotide sequences encoding the at least two gluten-specific TCRα or TCRβ amino acid sequences in the TCR dataset; or
   (ii) the frequency of T-cells expressing the nucleotide sequences encoding the at least two gluten-specific TCRα or TCRβ amino acid sequences in the sample; and, optionally,
e) comparing said normalised score to a defined threshold.

In a related aspect, provided herein is a method for detecting TCR sequences in cells in a sample, said method comprising the steps:
a) obtaining a sample comprising T-cells from a human subject;
b) isolating nucleic acids from the sample;
c) sequencing nucleotide sequences which encode TCRα chains and nucleotide sequences which encode TCRβ chains to provide a TCR dataset;
d) assigning a score to the TCR dataset, wherein said score is determined by the abundance in the dataset of nucleotide sequences which encode at least two gluten-specific TCRα or TCRβ amino acid sequences, wherein said at least two gluten-specific TCRα or TCRβ amino acid sequences comprise:
   (i) at least one TCRα or TCRβ amino acid sequence selected from SEQ ID NOs: 1 to 50; and
   (ii) at least one TCRα or TCRβ amino acid sequence selected from SEQ ID NOs: 51 to 432;
e) normalising said score to provide a normalised score representative of:
   (i) the frequency of the nucleotide sequences encoding the at least two gluten-specific TCRα or TCRβ amino acid sequences in the TCR dataset; or
   (ii) the frequency of T-cells expressing the nucleotide sequences encoding the at least two gluten-specific TCRα or TCRβ amino acid sequences in the sample; and, optionally
f) comparing said normalised score to a defined threshold.

In another aspect, provided herein is a composition suitable for multiplex PCR comprising a plurality of nucleic acid primers, wherein the composition comprises:
(i) primers able to specifically hybridise to the TCR V-gene segments specified in Table 1 and Table 2; and
(ii) primers able to specifically hybridise to the TCR J-gene segments specified in Table 1 and Table 2 or primers able to specifically hybridise to a nucleotide sequence encoding a TCR constant region;
wherein a primer of part (i) and a primer of part (ii) may be used in combination to generate an amplification product.

### BRIEF DESCRIPTION OF THE FIGURES

FIG. 1 shows the most frequent public TCRα sequences in 17 CD patients.

FIG. 2 shows the most frequent public TCRβ sequences in 17 CD patients.

FIG. 3 and FIG. 4 show the number of public TCRα and TCRβ sequences, respectively, that were found in the number of patients plotted on the y-axis. Gray bars show public TCRα or TCRβ sequences defined as identical amino acid sequences whereas open bars show semipublic TCRα and TCRβ motifs generated by collapsing TCRα or TCRβ amino acid sequences that differ by three residues or less. The top four CDR3α and the top five CDR3β motifs are shown in respective panels.

FIG. 5 shows overlap of TCRβ clonotypes at baseline, day 6 and day 14 or day 28 of the gluten challenge in patients CD442 and CD1300. The percentage in the lower left boxes denotes the proportion of shared clonotypes in the latest sample while the percentage in the upper right boxes denotes the proportion of shared clonotypes in the earliest sample. The TCRβ clonotypes were obtained from compilation of both single-cell and bulk sequencing data.

FIG. 6 shows significantly different scores between controls and untreated celiac disease (UCD) patients when the test is performed as described in Example 4. If a cut-off value is set to 3, all of the controls will test negative while 5 of seven UCD patients will test positive.

### DETAILED DESCRIPTION

The clear HLA association of the condition, the existence of T-cells that recognise gluten epitopes in the context of disease-associated HLA-DQ allotypes and the extraordinary performance of disease-relevant HLA:gluten peptide tetramers in the identification of T-cells which recognise gluten epitopes (Sarna, V. K. et al., supra), together identify celiac disease (CD) as an ideal model disorder in which to characterise the dynamics of pathogenic T-cells in a human HLA-associated disorder. By studying patients at different stages of disease and patients undergoing oral gluten challenge, the inventors have found that the clonotypes of gluten-specific T-cells are shared between the gut and blood compartments of an individual, that the recall response to gluten is dominated by expansion of pre-existing memory T-cells and that T-cell clonotypes persist for decades with no appreciable recruitment of new clonotypes to the repertoire. The inventors also found that about 10% of the TCRα, TCRβ or paired TCRβ sequences are publicly used in the response to gluten. The findings demonstrate that in an HLA-associated disease, after antigen sensitisation, patients are marked with permanent and stable immunological scars of disease-driving T-cells.

As used herein, the term “public TCR” indicates a TCR sequence, or a TCR having CDR sequences, shared between multiple individuals. Thus a celiac disease-associated public TCR is a TCR which is found in multiple individuals who suffer from celiac disease. More particularly, as used herein a public TCR is a TCR having a CDR3 amino acid sequence in a particular VJ gene context, which CDR3 sequence in which VJ gene context is found in multiple individuals who suffer from celiac disease. Accordingly, celiac disease-associated public TCRs may be considered as markers for celiac disease. Conversely, a “private TCR” is a TCR which is specific to a particular individual (i.e. it is not found in multiple individuals). In the context of celiac disease, a private TCR may be gluten-specific and contribute to the disease pathology, but is not considered a diagnostic marker for celiac disease because it is not found across the celiac disease patient group.

The inventors' work was made possible by combining tetramer-based cell isolation (Sarna, V. K. et al., supra) with high-throughput sequencing of the TCRα and TCRβ genes expressed by thousands of single cells and of bulk cell populations. Uniquely, the inventors had access to historic patient samples allowing them to assess the changes in the TCR repertoire of individual patients over decades. The inventors' conclusion is dependent on the high specificity of HLA-DQ2.5:gluten tetramer staining. Previously, the inventors found that 80% of HLADQ2.5: gluten tetramer-sorted T-cell clones cultured in vitro from celiac patients showed an antigen-specific proliferative response (Christophersen, A. et al., United European Gastroenterol. J. 2(4): 268-278, 2014). For single-cell data, the inventors rigorously analysed identical paired TCRαβ nucleotide sequences for clonotype assignment. The few cases of identical paired TCRαβ nucleotide sequences across individuals in the single-cell data originated from different sequencing libraries prepared and analysed months apart and thus represent a truly public response. Therefore, the extensive clonotype sharing the inventors have found in samples from the same individuals is not caused by cross contamination. Based on these findings, a non-invasive method for diagnosing celiac disease is provided.

The finding of the same T-cell clonotypes in samples collected decades apart raise the question how the clonotypes are preserved in the patients. Possibly, this could be due to longevity of memory cells. In the gut of humans, it was recently demonstrated that plasma cells may survive for decades. Even though long-lived memory CD4+ T-cells have been described in humans, it might be that gluten antigen challenge due to dietary transgressions contributes to the maintenance of the T-cell clonotypes in CD. The inventors observed upon oral gluten challenge in patients in remission that the majority of expanded clonotypes found at peak recall response were present prior to challenge as expanded populations of memory T-cells. Moreover, the majority of T-cell clonotypes observed in the gut lesion following challenge were identical to those circulating in blood at peak response suggesting that these clonotypes dominate the recall response.

Single and bulk populations of HLA-DQ:gluten tetramer-sorted CD4+ T-cells were analysed by high-throughput DNA sequencing of rearranged T-cell receptor α- and β-genes. Blood and gut biopsy samples from 21 celiac disease patients, taken at various stages of disease and with intervals of weeks to decades apart, were examined. Persistence of the same clonotypes was seen in both compartments over decades with up to 53% overlap between samples obtained 16-28 years apart. Further, the inventors observed that the recall response following oral gluten challenge is dominated by pre-existing CD4+ T-cell clonotypes. Public features were frequent among gluten-specific T-cells as 10% of TCRα, TCRβ or paired TCRαβ amino acid sequences of a total of 1813 TCRs isolated from 17 patients were observed in >2 patients. In established celiac disease, the T-cell clonotypes that recognise gluten are persistent for decades, making up fixed repertoires that prevalently exhibit public features.

As T-cells recognise peptide antigen with their T-cell receptor (TCR) in the context of MHC (HLA in human) molecules, T-cells very likely play a central role in HLA-associated disorders. Each naïve T-cell expresses a unique TCR as a result of gene recombination of different V, D and J germline segments and random deletion or insertion of non-germline nucleotides at the V(D)J junction. Upon antigen recognition by the TCRs, T-cells become activated, clonally expand and naïve T-cells change phenotype to become memory T-cells. The TCR repertoire is made up of the collective representation of unique TCRs. Technological developments have opened avenues to explore the TCR repertoire in infectious and autoimmune conditions with high throughput methods. Obviously, in HLA-associated disorders monitoring of the dynamics of pathogenic T-cells in time and body space will be of interest. This is however challenging, mainly due to difficulties in defining pathogenic T-cells, and no studies have so far investigated changes in the repertoires of antigen-specific and disease-relevant T-cells. By harnessing HLA-DQ:gluten tetramers relevant to celiac disease (CD) covering the immunodominant gluten epitopes (DQ2.5-glia-α1a, DQ2.5-glia-α2, DQ2.5-glia-ω1, DQ2.5-glia-ω2, DQ8-glia-α1 and DQ8-glia-γ1b) and undertaking large-scale TCR sequencing of HLA-DQ:gluten tetramer-binding cells, the inventors have performed a study addressing TCR repertoire dynamics and maintenance. CD is an autoimmune and inflammatory disease of the small intestine driven by gluten-specific CD4+ T-cells that recognise deamidated gluten peptides in the context of the disease-associated HLA-DQ2/8 molecules. The disease activity is controlled by dietary gluten exposure, and hence life-long gluten-free diet (GFD) is an effective treatment of the disease.

### Identical Gluten-Specific Clonotypes are Found in Peripheral Blood and Gut Mucosa.

The inventors sorted gluten-specific CD4+ T-cells binding to a pool of four HLA-DQ:gluten tetramers presenting the most immunodominant HLA-DQ2.5-restricted gluten epitopes from matched blood and gut biopsy samples from three untreated CD patients. While such tetramer-binding cells amount to around 2% of CD4+ T-cells in intestinal lamina propria of untreated patients, these cells are rare in blood, ranging from 3-70 cells per million CD4+ T-cells. Identical TCRβ clonotypes defined by unique nucleotide sequence were found in both sampled compartments. Because of sampling limitations, the maximum observed clonotype overlap between two independent sequencing experiments of the same sample was around 50% (95% CI, 42 to 59). Based on the high degree of clonotype sharing and the fact that the HLA-DQ:gluten tetramer-binding effector-memory T-cells in blood are gut homing, the inventors conclude that the more easily accessible gluten-specific T-cells in blood reflect the repertoire of the gluten-specific T-cells in gut.

### Frequency of Gluten-Specific CD4+ T-Cells Decrease Upon GFD

The inventors analysed gluten-specific T-cells in gut biopsies and in peripheral blood of six untreated celiac disease (UCD) patients who were followed up until 2 years after commencement of GFD. Upon commencement of GFD, the frequency of gluten-specific T-cells in blood decreased in all subjects, but at a variable rate. Most subjects had a clear decline by one year, except two subjects (CD1283 and CD1268) who showed a decrease in the frequency of gluten-specific CD4+ T-cells only at additional follow-up after two years of GFD. From all six patients, the inventors sorted circulating and gut tissue-resident gluten-specific CD4+ T-cells as single cells and performed paired TCRαβ sequencing. The inventors observed expansion of multiple clones in all samples. The extent of clonal dominance, calculated by the sample-corrected Shannon diversity index, was highest in UCD patients and decreased upon GFD. Thus, clonal contraction appears to be a major cause for the observed decrease in the frequency of circulating gluten-specific CD4+ T-cells upon GFD.

### The Same Clonotypes are Found in Multiple Samples Taken Weeks to Years Apart.

Next, the inventors studied whether cells of the same clonotype, defined as cells expressing an identical pairing of TCRαβ chains (i.e. expressing TCRα and TCRβ chains with identical amino acid sequences and encoded by identical DNA sequences), were present in samples taken at different timepoints from the same individual. Taking into account the repertoire diversity and the limited sampling (i.e. up to 100 ml blood amounting to <2% of total blood volume and 2-20 mm³ of intestinal tissue sampled from over 25 cm of duodenum) that resulted in less than 100 sequenced cells per sample, detection of cells of same clonotypes in multiple samples is not a given. Notwithstanding these facts, and very strikingly, the inventors found in all six patients the re-occurrence of many clonotypes in multiple samples. The proportion of clonotypes found after commencement of GFD that were also found in the first samples when the patients were untreated varied somewhat, likely due to limited sampling. More importantly, there is no trend of decreasing overlap over time. Since the patients were on GFD after the initial sampling point, new gluten-specific clonotypes should not be recruited from the naïve to the memory repertoire. Thus, after commencement of GFD, the clonally expanded gluten-specific T-cells contract and remain as memory T-cells.

### Gluten-Specific Memory T-Cells Expand and Dominate on Oral Gluten Challenge.

To study the impact of gluten antigen reintroduction on the gluten-specific T-cell repertoire, the inventors challenged treated CD patients with dietary gluten for 14 days. In seven participants who showed significant increase in the number of HLA-DQ:gluten tetramer-binding T-cells after gluten challenge, the inventors performed paired single-cell TCRαβ sequencing. Similarly to earlier findings, the gluten-specific T-cell repertoires were composed of clonally expanded cells from a diverse set of clonotypes. The degree of clonal expansion increased, as demonstrated by lower sample-corrected Shannon diversity index, in the circulating gluten-specific T-cells on day 6. Concurrently, the total number of circulating gluten-specific T-cells reached a peak level on day 6.

A major question raised by this challenge study is whether the gluten-specific T-cell response induced by re-exposure to gluten consists of re-activation of pre-existing memory T-cells or involves recruitment of naïve T-cells. When the inventors compared clonotypes sampled on day 6 with the baseline memory repertoire, we found a considerable overlap. These data suggest that the gluten-specific T-cell repertoire on day 6 is primarily made up of clonal expansion of pre-existing memory T-cells.

Unchanged Dominance of Memory Clonotypes 28 Days after Reintroduction of Gluten.

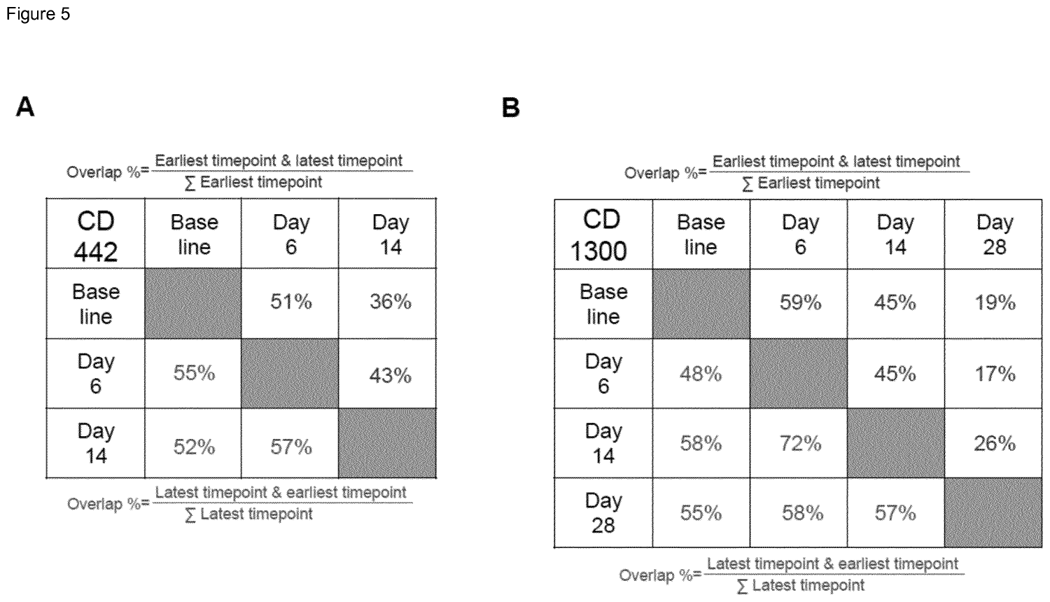

The inventors next compared paired nucleotide TCRβ clonotype data from blood and biopsy samples taken on day 14, or from an additional blood sample taken on day 28 after gluten challenge, with clonotype data at baseline. From the single-cell data of all seven patients, the inventors found that 12-44% of TCRαβ clonotypes detected at the latest timepoint were also found in the memory T-cell repertoire at baseline prior to challenge. To maximise the sample sizes, the inventors additionally performed bulk sequencing of samples from two patients who had many gluten-specific T-cells. With more clonotypes being detected by bulk sequencing, the inventors found that 52-55% of TCRβ clonotypes detected at the latest timepoint were present in the baseline samples. The proportion of clonotypes in samples taken at day 6, day 14 and day 28 that had already been observed at baseline remained remarkably stable (48-58%) with no indication of declining dominance of memory clonotypes over time (FIG. 5). The data suggests that re-introduction of gluten causes a transient clonal expansion of existing gluten-specific memory T-cells with no alteration of the overall gluten-specific T-cell repertoire and with no apparent sign of recruitment of new clonotypes from the naïve repertoire.

### Similar Fraction of Clonotypes is Observed 6 Months and 27 Years Apart.

Patients in the challenge study were followed for only up to 28 days. It is possible that the gluten-specific T-cell repertoire changes slowly, or only after repeated gluten antigen exposure. To compare TCR repertoire many years apart, the inventors invited five patients, from whom historic T-cell material from decades ago was available, to donate new blood and biopsy samples. Using single-cell sequencing, paired TCRαβ clonotype sharing on the nucleotide level was observed, including identical nucleotide sequences of secondary productive TCRα chains, between historic and recent samples, but to a variable degree. For patients CD373 and CD412 the inventors only had access to very small cryopreserved samples from the 1990s, in which the sharing was low (2-4%). However, when the sample size from CD412 was increased by bulk sequencing of an in vitro-expanded T-cell line from a single biopsy specimen, the overlap increased to 18%. For CD114, who was diagnosed in his early childhood, the inventors had two historic samples from the 1980s that were taken 19.5 and 20 years after his diagnosis and commencement of the GFD. These two samples taken six months apart had 51 clonotypes in common, which made up 71% of the smaller 19.5 year GFD sample (total of 72 clonotypes), but only 19% of the much larger (n=264) 20 year GFD sample. Interestingly, the inventors found a similar degree of TCRβ clonotype overlap in the recent samples taken 47 years after diagnosis with the previous samples taken more than two decades ago (22-53%). Identical clonotypes, especially those with the largest clonal sizes, were also observed in samples taken 16-20 years apart in the remaining two patients. Taking the limited sampling from a diverse repertoire into account, the inventors conclude that the gluten-specific T-cell repertoire in CD patients remains remarkably stable over several decades.

### 10% of Gluten-Specific T-Cells Use Public TCR Sequences

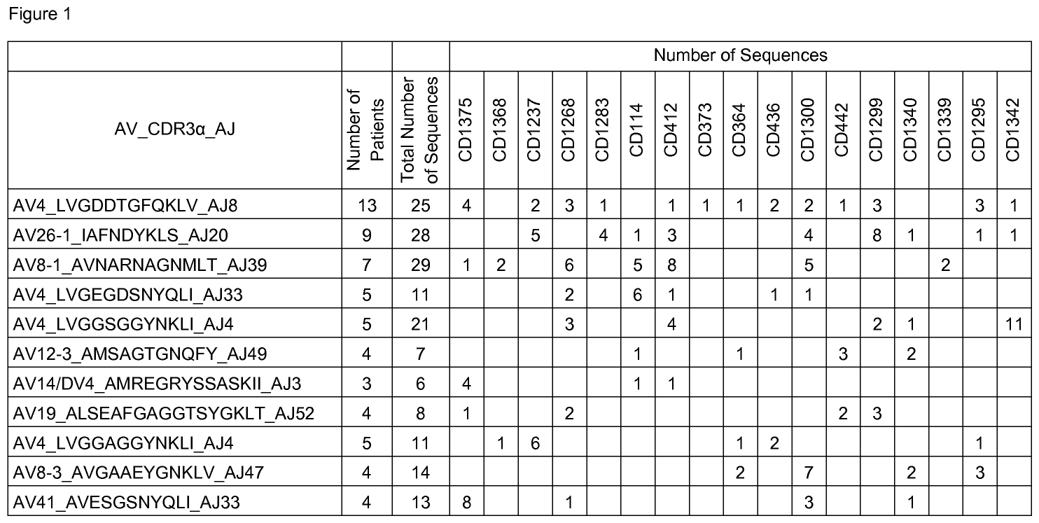

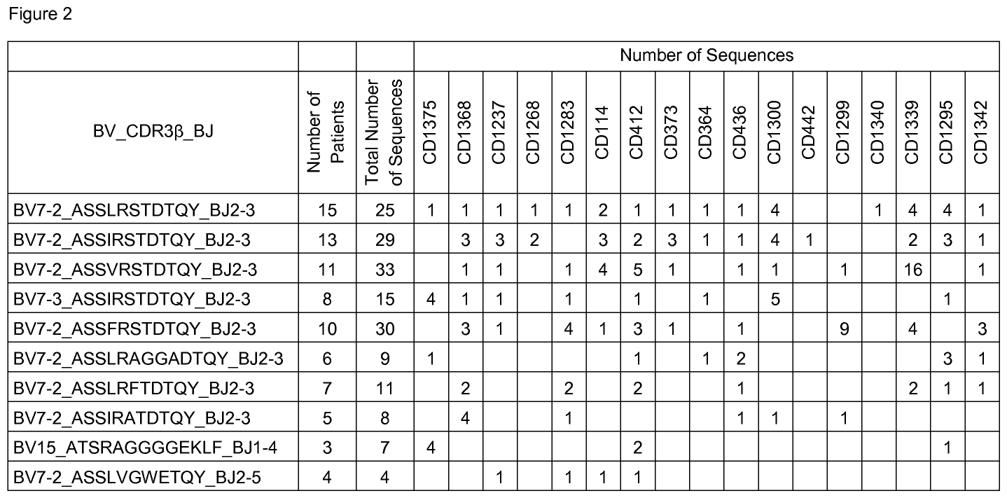

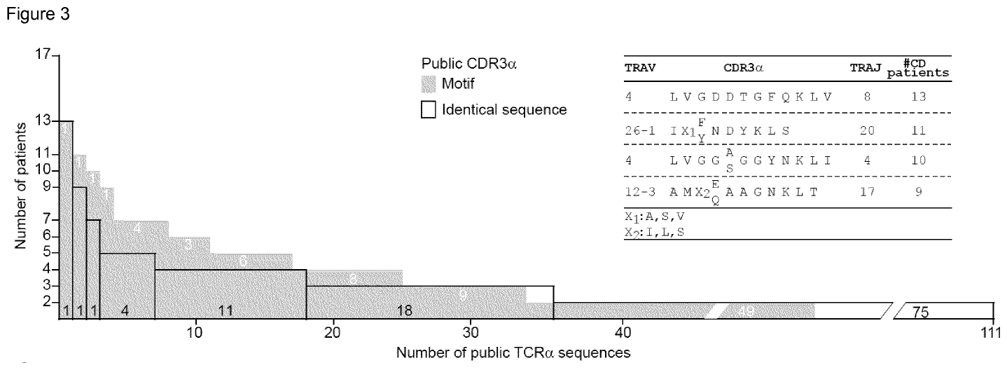

The inventors collected a total of 1813 unique paired amino acid TCRαβ sequences from 17 HLADQ2.5+CD patients by single-cell TCR sequencing. Within this dataset, the inventors frequently observed identical amino acid sequences for either TCRα or TCRβ chain in different individuals (FIG. 1 and FIG. 2). Closer inspection of these public TCR sequences revealed common CDR3 motifs. The inventors collapsed public TCR sequences that used the same V- and J-gene segment, had the same CDR3 length and differed by no more than three amino acids in the CDR3 sequences to generate a list of public TCR sequences (Table 3). In addition, the inventors identified 40 paired public TCRαβ sequences where identical amino acid TCRαβ sequences were found among cells from 2-4 individuals. In most cases, this public response is a result of convergent recombination where each individual expresses unique nucleotide sequences that converge toward identical amino acid sequences. In total, there were 229 publicly used TCRα, TCRβ or paired TCRαβ sequences amounting to 10% of all paired TCRαβ amino acid sequences in this study.

CD-associated TCR sequences for use in the present invention are set forth in the tables below. The tables disclose TCR sequences defined based on the V-gene and J-gene which encode them, and the CDR3 amino acid sequence. The disclosed information is in a standard format well understood by the skilled person and sufficient for the skilled person to determine the entire sequence of the TCR chain variable region. The sequences of the TCR α- and β-chain constant regions are also well known in the art, so the skilled person may easily deduce from the information below the entire sequence of each listed TCR chain. It is to be understood that the SEQ ID NOs listed in the tables below refer to the entire TCR chains as defined by the CDR3 sequence, and the V and J genes, and not simply the listed CDR3 sequences. More particularly, in the sequence listing the SEQ ID NOs refer to the entire TCR variable regions comprising the V segment, CDR3 sequence and J segment.

The majority of TCRs are heterodimeric receptors comprising an alpha chain and a beta chain, each comprising a variable domain and a constant domain. Both types of chains comprise three complementarity-determining regions (CDRs): CDR1, CDR2 and CDR3. During T-cell development, TCR genes undergo a sequence of ordered recombination events involving variable (V), joining (J), and in some cases, diversity (D) gene segments. The TCR alpha chain gene is generated by VJ recombination, whereas the beta chain gene is generated by VDJ recombination. The nucleotide sequences of CDR3 are generated by somatic recombination of segregated germline variable (V), diversity (D), and joining (J) gene segments for the TCR β chain (TRB), and V and J gene segments for the TCR α chain (TRA). It generally accepted that the antigenic specificity of T-cells is mainly determined by the amino acid sequences of the CDR3s. The human TRA locus at 14q11.2 spans 1000 kilobases (kb). It comprises 54 TRAV genes belonging to 41 subgroups, 61 TRAJ segments localized on 71 kb, and a unique TRAC gene. The human TRB locus at 7q35 spans 620 kb.

It comprises 64-67 TRBV genes belonging to 32 subgroups. Except for TRBV30, localised downstream of the TRBC2 gene, in inverted orientation for transcription, all the other TRBV genes are located upstream of a duplicated D-J-C-cluster, which comprises, in the first part, one TRBD, six TRBJ, and the TRBC1 gene, and in the second part, one TRBD, eight TRBJ, and the TRBC2 gene. The genomic source, i.e. gene segments, of the alpha chains and beta chains identified as celiac disease-associated public TCR sequences are indicated in Tables 1 to 3, which together with the amino acid sequence of CDR3 unambiguously specify the amino acid sequence of the TCR chain.

TABLE 1
Previously-known CD-associated TCRα and TCRβ chain sequences:

| SEQ | | | | |
|---|---|---|---|---|
| ID NO | V-Gene | CDR3 sequence | J-Gene | Reference |
| 1 | TRAV26-1 | IAFNDYKLS | TRAJ20 | Qiao 2014, PMID 24038601 |
| 2 | TRAV26-1 | IAYNDYKLS | TRAJ20 | Qiao 2014, PMID 24038601 |
| 3 | TRAV26-1 | IVFGGSQGNLI | TRAJ42 | Qiao 2014, PMID 24038601 |
| 4 | TRAV26-1 | IVFNDYKLS | TRAJ20 | Qiao 2014, PMID 24038601 |
| 5 | TRAV26-1 | IVYGGSQGNLI | TRAJ42 | Qiao 2014, PMID 24038601 |
| 6 | TRAV26-1 | IVYNDYKLS | TRAJ20 | Qiao 2014, PMID 24038601 |
| 7 | TRAV35 | AGPYNTDKLI | TRAJ34 | Petersen 2014, PMID 24777060 |
| 8 | TRAV4 | LVGVMEYGNKLV | TRAJ47 | Dahal-Koirala 2016, PMID 26838051 |
| 9 | TRBV29-1 | SAGQGGTGELF | TRBJ2-2 | Petersen 2014, PMID 24777060 |
| 10 | TRBV5-1 | ASSFDGETQY | TRBJ2-5 | Yohannes 2017, PMID 29269859 |
| 11 | TRBV5-1 | ASSLGQPSTDTQY | TRBJ2-3 | WO 2014/179202 (sequence 8) |
| 12 | TRBV6-1 | ASFLGPVFPGGYT | TRBJ1-2 | Dahal-Koirala 2016, PMID 26838051 |
| 13 | TRBV7-2 | ASSLVGWETQY | TRBJ2-5 | Qiao 2011, PMID 21849672 |
| 14 | TRBV7-3 | ASSLNWDTEAF | TRBJ1-1 | Petersen 2014, PMID 24777060 |
| 15 | TRBV7-6 | ASSLASAGGTDTQY | TRBJ2-3 | Petersen 2014, PMID 24777060 |
| 16 | TRBV7-8 | ASSLNWDTEAF | TRBJ1-1 | Yohannes 2017, PMID 29269859 |
| 17 | TRBV7-2 | ASSFRHTDTQY | TRBJ2-3 | Qiao 2011, PMID 21849672 |
| 18 | TRBV7-2 | ASSFRSTDTQY | TRBJ2-3 | Qiao 2011, PMID 21849672 |
| 19 | TRBV7-2 | ASSFRTTDTQY | TRBJ2-3 | Qiao 2011, PMID 21849672 |
| 20 | TRBV7-2 | ASSFRYTDTQY | TRBJ2-3 | Qiao 2011, PMID 21849672 |
| 21 | TRBV7-2 | ASSIRATDTQY | TRBJ2-3 | Qiao 2011, PMID 21849672 |
| 22 | TRBV7-2 | ASSIRDTDTQY | TRBJ2-3 | Qiao 2011, PMID 21849672 |
| 23 | TRBV7-2 | ASSIRFTDTQY | TRBJ2-3 | Qiao 2011, PMID 21849672 |
| 24 | TRBV7-2 | ASSIRGTDTQY | TRBJ2-3 | Qiao 2011, PMID 21849672 |
| 25 | TRBV7-2 | ASSIRHTDTQY | TRBJ2-3 | Qiao 2011, PMID 21849672 |
| 26 | TRBV7-2 | ASSIRLTDTQY | TRBJ2-3 | Qiao 2011, PMID 21849672 |
| 27 | TRBV7-2 | ASSIRSTDTQY | TRBJ2-3 | Qiao 2011, PMID 21849672 |
| 28 | TRBV7-2 | ASSIRVTDTQY | TRBJ2-3 | Qiao 2011, PMID 21849672 |
| 29 | TRBV7-2 | ASSIRYTDTQY | TRBJ2-3 | Qiao 2011, PMID 21849672 |
| 30 | TRBV7-2 | ASSLRATDTQY | TRBJ2-3 | Qiao 2011, PMID 21849672 |
| 31 | TRBV7-2 | ASSLRFTDTQY | TRBJ2-3 | Qiao 2011, PMID 21849672 |
| 32 | TRBV7-2 | ASSLRHTDTQY | TRBJ2-3 | Qiao 2011, PMID 21849672 |
| 33 | TRBV7-2 | ASSLRSTDTQY | TRBJ2-3 | Qiao 2011, PMID 21849672 |
| 34 | TRBV7-2 | ASSLRWTDTQY | TRBJ2-3 | Qiao 2011, PMID 21849672 |
| 35 | TRBV7-2 | ASSLRYTDTQY | TRBJ2-3 | Qiao 2011, PMID 21849672 |
| 36 | TRBV7-2 | ASSVRFTDTQY | TRBJ2-3 | Qiao 2011, PMID 21849672 |
| 37 | TRBV7-2 | ASSVRSTDTQY | TRBJ2-3 | Qiao 2011, PMID 21849672 |
| 38 | TRBV7-2 | ASSVRYTDTQY | TRBJ2-3 | Qiao 2011, PMID 21849672 |
| 39 | TRBV7-2 | ASSYRSTDTQY | TRBJ2-3 | Qiao 2011, PMID 21849672 |
| 40 | TRBV7-3 | ASSFRSTDTQY | TRBJ2-3 | Gunnarsen 2017, PMID 28878121 |
| 41 | TRBV7-3 | ASSIRATDTQY | TRBJ2-3 | Gunnarsen 2017, PMID 28878121 |
| 42 | TRBV7-3 | ASSIRGTDTQY | TRBJ2-3 | Gunnarsen 2017, PMID 28878121 |
| 43 | TRBV7-3 | ASSIRSTDTQY | TRBJ2-3 | Gunnarsen 2017, PMID 28878121 |
| 44 | TRBV7-3 | ASSLRATDTQY | TRBJ2-3 | Gunnarsen 2017, PMID 28878121 |
| 45 | TRBV7-3 | ASSLRHTDTQY | TRBJ2-3 | Gunnarsen 2017, PMID 28878121 |
| 46 | TRBV7-3 | ASSLRSTDTQY | TRBJ2-3 | Gunnarsen 2017, PMID 28878121 |
| 47 | TRBV7-3 | ASSVRATDTQY | TRBJ2-3 | Gunnarsen 2017, PMID 28878121 |
| 48 | TRBV7-3 | ASSVRSTDTQY | TRBJ2-3 | Gunnarsen 2017, PMID 28878121 |
| 49 | TRBV7-2 | ASSxRxTDTQY | TRBJ2-3 | Qiao 2011, PMID 21849672 |
| 50 | TRBV7-3 | ASSxRxTDTQY | TRBJ2-3 | Gunnarsen 2017, PMID 28878121 |

TABLE 2
Newly-identified CD-associated
TCRα and TCRβ chain sequences:

| SEQ ID NO | V-Gene | CDR3 sequence | J-Gene |
|---|---|---|---|
| 51 | TRAV1-2 | AVRAVFSGGYNKLI | TRAJ4 |
| 52 | TRAV1-2 | AVRAVLSGGYNKLI | TRAJ4 |
| 53 | TRAV1-2 | AVRAVVSGGYNKLI | TRAJ4 |
| 54 | TRAV1-2 | AVTSSNTGKLI | TRAJ37 |
| 55 | TRAV1-2 | AVTTSNTGKLI | TRAJ37 |
| 56 | TRAV12-1 | VVNLYSSASKII | TRAJ3 |
| 57 | TRAV12-1 | VVNNASSASKII | TRAJ3 |
| 58 | TRAV12-1 | VVNQYSSASKII | TRAJ3 |
| 59 | TRAV12-1 | VVNSASSASKII | TRAJ3 |
| 60 | TRAV12-1 | VVTLMDTGRRALT | TRAJ5 |
| 61 | TRAV12-2 | APQGATNKLI | TRAJ32 |
| 62 | TRAV12-2 | ASQDTGRRALT | TRAJ5 |
| 63 | TRAV12-2 | AVATYNFNKFY | TRAJ21 |
| 64 | TRAV12-2 | AVFPGGATNKLI | TRAJ32 |
| 65 | TRAV12-2 | AVKDSSASKII | TRAJ3 |
| 66 | TRAV12-2 | AVNMFSGGYNKLI | TRAJ4 |
| 67 | TRAV12-2 | AVNMNYGGATNKLI | TRAJ32 |
| 68 | TRAV12-2 | AVPNRDDKII | TRAJ30 |
| 69 | TRAV12-2 | AVSNRDDKII | TRAJ30 |
| 70 | TRAV12-3 | AAPQGGSEKLV | TRAJ57 |
| 71 | TRAV12-3 | AIYTGTASKLT | TRAJ44 |
| 72 | TRAV12-3 | AMIEAAGNKLT | TRAJ17 |
| 73 | TRAV12-3 | AMIQAAGNKLT | TRAJ17 |
| 74 | TRAV12-3 | AMKDYGQNFV | TRAJ26 |
| 75 | TRAV12-3 | AMLEAAGNKLT | TRAJ17 |
| 76 | TRAV12-3 | AMNDYGNNRLA | TRAJ7 |
| 77 | TRAV12-3 | AMRDYGQNFV | TRAJ26 |
| 78 | TRAV12-3 | AMSAGTGNQFY | TRAJ49 |
| 79 | TRAV12-3 | AMSASSGGGADGLT | TRAJ45 |
| 80 | TRAV12-3 | AMSDLPGGSNYKLT | TRAJ53 |
| 81 | TRAV12-3 | AMSEAAGNKLT | TRAJ17 |
| 82 | TRAV12-3 | AMSEGTGNQFY | TRAJ49 |
| 83 | TRAV12-3 | AMSEIPGGSNYKLT | TRAJ53 |
| 84 | TRAV12-3 | AMSELPGGSNYKLT | TRAJ53 |
| 85 | TRAV12-3 | AMTDYGNNRLA | TRAJ7 |
| 86 | TRAV13-1 | AASNTDKLI | TRAJ34 |
| 87 | TRAV13-2 | AEGDAGGTSYGKLT | TRAJ52 |
| 88 | TRAV13-2 | AETNAGGTSYGKLT | TRAJ52 |
| 89 | TRAV14/DV4 | AMNTGGFKTI | TRAJ9 |
| 90 | TRAV14/DV4 | AMREEGSQGNLI | TRAJ42 |
| 91 | TRAV14/DV4 | AMREGRYSSASKII | TRAJ3 |
| 92 | TRAV16 | ALNSGGYQKVT | TRAJ13 |
| 93 | TRAV16 | ALSAPINYQLI | TRAJ33 |
| 94 | TRAV16 | ALSDSNYQLI | TRAJ33 |
| 95 | TRAV17 | ATDAETSGSRLT | TRAJ58 |
| 96 | TRAV17 | ATDDKGGSEKLV | TRAJ57 |
| 97 | TRAV17 | ATEGNTGFQKLV | TRAJ8 |
| 98 | TRAV19 | ALSEAFGAGGTSYGKLT | TRAJ52 |
| 99 | TRAV19 | ALSEAGANSKLT | TRAJ56 |
| 100 | TRAV19 | ALSEGGFGNVLH | TRAJ35 |
| 101 | TRAV19 | ALSEGGNAGNMLT | TRAJ39 |
| 102 | TRAV19 | ALSEGGNQGGKLI | TRAJ23 |
| 103 | TRAV19 | ALSEGSNAGNMLT | TRAJ39 |
| 104 | TRAV19 | ALSGAGANSKLT | TRAJ56 |
| 105 | TRAV19 | ALSGGGANSKLT | TRAJ56 |
| 106 | TRAV19 | ALTLNRDDKII | TRAJ30 |
| 107 | TRAV2 | AVEDLRAGSYQLT | TRAJ28 |
| 108 | TRAV2 | AVEVYNFNKFY | TRAJ21 |
| 109 | TRAV20 | AVQGDRLTGGGNKLT | TRAJ 10 |
| 110 | TRAV21 | AVPSGAGSYQLT | TRAJ28 |
| 111 | TRAV21 | AVTGTYKYI | TRAJ40 |
| 112 | TRAV22 | AVELQGAQKLV | TRAJ54 |
| 113 | TRAV22 | AVERADSWGKLQ | TRAJ24 |
| 114 | TRAV22 | AVERQGAQKLV | TRAJ54 |
| 115 | TRAV23/DV6 | AASSAGGTSYGKLT | TRAJ52 |
| 116 | TRAV26-1 | IAPSGTYKYI | TRAJ40 |
| 117 | TRAV26-1 | IDPGSSNTGKLI | TRAJ37 |
| 118 | TRAV26-1 | IGNYGGSQGNLI | TRAJ42 |
| 119 | TRAV26-1 | IPNYGGSQGNLI | TRAJ42 |
| 120 | TRAV26-1 | ISFNDYKLS | TRAJ20 |
| 121 | TRAV26-1 | IVFNARLM | TRAJ31 |
| 122 | TRAV26-1 | IVHNARLM | TRAJ31 |
| 123 | TRAV26-1 | IVLGGATNKLI | TRAJ32 |
| 124 | TRAV26-1 | IVLNARLM | TRAJ31 |
| 125 | TRAV26-1 | IVPPGTASKLT | TRAJ44 |
| 126 | TRAV26-1 | IVPQGAQKLV | TRAJ54 |
| 127 | TRAV26-1 | IVRVVGDDKII | TRAJ30 |
| 128 | TRAV26-1 | IVTDGQKLL | TRAJ16 |
| 129 | TRAV26-1 | IVTGNQFY | TRAJ49 |
| 130 | TRAV26-1 | IVTSGSRLT | TRAJ58 |
| 131 | TRAV26-1 | IVYGGSEKLV | TRAJ57 |
| 132 | TRAV26-1 | IVYNARLM | TRAJ31 |
| 133 | TRAV26-1 | IVYNNDMR | TRAJ43 |
| 134 | TRAV26-1 | IVYNTDKLI | TRAJ34 |
| 135 | TRAV26-1 | IVYSGNTPLV | TRAJ29 |
| 136 | TRAV27 | AGEGNAGGTSYGKLT | TRAJ52 |
| 137 | TRAV29/DV5 | AASADAGGTSYGKLT | TRAJ 52 |
| 138 | TRAV29/DV5 | AASAGETSGSRLT | TRAJ58 |
| 139 | TRAV29/DV5 | AASALTSGTYKYI | TRAJ40 |
| 140 | TRAV29/DV5 | AASEETSGSRLT | TRAJ58 |
| 141 | TRAV29/DV5 | AASEQSGGSNYKLT | TRAJ53 |
| 142 | TRAV29/DV5 | AASGGGGSTLGRLY | TRAJ18 |
| 143 | TRAV29/DV5 | AASVATDSWGKLQ | TRAJ24 |
| 144 | TRAV29/DV5 | AASVLYGSSNTGKLI | TRAJ37 |
| 145 | TRAV29/DV5 | AATNTNAGKST | TRAJ27 |
| 146 | TRAV3 | AVRDGYGNNRLA | TRAJ7 |
| 147 | TRAV3 | RTLT | TRAJ11 |
| 148 | TRAV34 | GADQGAQKLV | TRAJ54 |
| 149 | TRAV35 | AANDYKLS | TRAJ20 |
| 150 | TRAV35 | AATTGGSQGNLI | TRAJ42 |
| 151 | TRAV35 | AGDSGGGADGLT | TRAJ45 |
| 152 | TRAV35 | AGDSNYQLI | TRAJ33 |
| 153 | TRAV35 | AGFNTDKLI | TRAJ34 |
| 154 | TRAV35 | AGGNDYKLS | TRAJ20 |
| 155 | TRAV35 | AGHNTDKLI | TRAJ34 |
| 156 | TRAV35 | AGNDYKLS | TRAJ20 |
| 157 | TRAV35 | AGNYGGATNKLI | TRAJ32 |
| 158 | TRAV35 | AGQLDSGTYKYI | TRAJ40 |
| 159 | TRAV35 | AGQLGGATNKLI | TRAJ32 |
| 160 | TRAV35 | AGQLNAGGTSYGKLT | TRAJ52 |
| 161 | TRAV35 | AGQPGSSNTGKLI | TRAJ37 |
| 162 | TRAV35 | AGQQGAQKLV | TRAJ54 |
| 163 | TRAV35 | AGQVGSSNTGKLI | TRAJ37 |
| 164 | TRAV35 | AGVYNNNDMR | TRAJ43 |
| 165 | TRAV38-1 | AFTVYTGANSKLT | TRAJ56 |
| 166 | TRAV38-2/DV8 | AYRSTRYNNNDMR | TRAJ43 |
| 167 | TRAV38-2/DV8 | AYRTTRYGQNFV | TRAJ26 |
| 168 | TRAV39 | AVDPGYALN | TRAJ41 |
| 169 | TRAV4 | LVDNAGNMLT | TRAJ39 |
| 170 | TRAV4 | LVGDDTGFQKLV | TRAJ8 |
| 171 | TRAV4 | LVGDENTGTASKLT | TRAJ44 |
| 172 | TRAV4 | LVGDETGGYNKLI | TRAJ4 |
| 173 | TRAV4 | LVGDGDGGATNKLI | TRAJ32 |
| 174 | TRAV4 | LVGDGGGYNKLI | TRAJ4 |
| 175 | TRAV4 | LVGDPTGFQKLV | TRAJ8 |
| 176 | TRAV4 | LVGEGDSNYQLI | TRAJ33 |
| 177 | TRAV4 | LVGGAGGYNKLI | TRAJ4 |
| 178 | TRAV4 | LVGGDNQGGKLI | TRAJ23 |
| 179 | TRAV4 | LVGGDSSYKLI | TRAJ12 |
| 180 | TRAV4 | LVGGGGGADGLT | TRAJ45 |
| 181 | TRAV4 | LVGGHGSSNTGKLI | TRAJ37 |
| 182 | TRAV4 | LVGGSGGYNKLI | TRAJ4 |
| 183 | TRAV4 | LVGGYNNNDMR | TRAJ43 |
| 184 | TRAV4 | LVGQNFGNEKLT | TRAJ48 |
| 185 | TRAV4 | LVGTLTGGGNKLT | TRAJ10 |
| 186 | TRAV41 | AVAGTASKLT | TRAJ44 |
| 187 | TRAV41 | AVEAGSNYQLI | TRAJ33 |
| 188 | TRAV41 | AVEGGSNYKLT | TRAJ53 |
| 189 | TRAV41 | AVESGSNYQLI | TRAJ33 |
| 190 | TRAV41 | AVETSGSRLT | TRAJ58 |
| 191 | TRAV41 | AVEWGSNYQLI | TRAJ33 |
| 192 | TRAV5 | AEAGGGNKLT | TRAJ10 |
| 193 | TRAV5 | AESKSGGYNKLI | TRAJ4 |
| 194 | TRAV6 | ALPSGYALN | TRAJ41 |
| 195 | TRAV6 | ALSTDSWGKLQ | TRAJ24 |
| 196 | TRAV8-1 | AVNARNAGNMLT | TRAJ39 |
| 197 | TRAV8-1 | AVNARNSGYALN | TRAJ41 |
| 198 | TRAV8-1 | AVNRNTGFQKLV | TRAJ8 |
| 199 | TRAV8-2 | ASLSNFGNEKLT | TRAJ48 |
| 200 | TRAV8-2 | AVSEWAGNQFY | TRAJ49 |
| 201 | TRAV8-3 | AVATDRGSTLGRLY | TRAJ18 |
| 202 | TRAV8-3 | AVGAAEYGNKLV | TRAJ47 |
| 203 | TRAV8-3 | AVGASEYGNKLV | TRAJ47 |
| 204 | TRAV8-3 | AVGAVEYGNKLV | TRAJ47 |
| 205 | TRAV8-3 | AVGLDRGSTLGRLY | TRAJ18 |
| 206 | TRAV8-3 | AVGLTDSWGKLQ | TRAJ24 |
| 207 | TRAV8-3 | AVGPAEYGNKLV | TRAJ47 |
| 208 | TRAV8-3 | AVGSDRGSTLGRLY | TRAJ18 |
| 209 | TRAV8-3 | AVGTDRGSTLGRLY | TRAJ18 |
| 210 | TRAV8-3 | AVGVDRGSTLGRLY | TRAJ18 |
| 211 | TRAV8-3 | AVGVSEYGNKLV | TRAJ47 |
| 212 | TRAV8-3 | AVVHSSYKLI | TRAJ12 |
| 213 | TRAV9-2 | ALAEYNFNKFY | TRAJ21 |
| 214 | TRAV9-2 | ALSDGSGAGSYQLT | TRAJ28 |
| 215 | TRAV9-2 | ALSDPTGANSKLT | TRAJ56 |
| 216 | TRAV9-2 | ALSDPTGTASKLT | TRAJ44 |
| 217 | TRAV9-2 | ALSDQDTGRRALT | TRAJ5 |
| 218 | TRAV9-2 | ALSDQTGANNLF | TRAJ36 |
| 219 | TRAV9-2 | ALSDQTGTASKLT | TRAJ44 |
| 220 | TRAV9-2 | ALSEGNFNKFY | TRAJ21 |
| 221 | TRAV9-2 | ALSGGTSYGKLT | TRAJ52 |
| 222 | TRAV9-2 | ALSGSAGGTSYGKLT | TRAJ52 |
| 223 | TRBV10-3 | AISASGTEAF | TRBJ1-1 |
| 224 | TRBV11-2 | ASSSTAQETQY | TRBJ2-5 |
| 225 | TRBV12-3 | ASRLTLGTDTQY | TRBJ2-3 |
| 226 | TRBV12-3 | ASRPRGAPSYEQY | TRBJ2-7 |
| 227 | TRBV12-3 | ASSWTSWDTQY | TRBJ2-3 |
| 228 | TRBV15 | ATSRAGGGGEKLF | TRBJ1-4 |
| 229 | TRBV18 | ASSLAGWDTEAF | TRBJ1-1 |
| 230 | TRBV18 | ASSPAGWDTEAF | TRBJ1-1 |
| 231 | TRBV19 | AISTQGGNEQF | TRBJ2-1 |
| 232 | TRBV19 | ASSIFSLAGASYNEQF | TRBJ2-1 |
| 233 | TRBV19 | ASSIGTSGETQY | TRBJ2-5 |
| 234 | TRBV19 | ASSIRTGGSEQY | TRBJ2-7 |
| 235 | TRBV19 | ASSIVGGADQPQH | TRBJ1-5 |
| 236 | TRBV19 | ASSIVGSGGYNEQF | TRBJ2-1 |
| 237 | TRBV19 | ASSTGTSGETQY | TRBJ2-5 |
| 238 | TRBV20-1 | SAESGYNEQF | TRBJ2-1 |
| 239 | TRBV20-1 | SAKPPTGDFSYEQY | TRBJ2-7 |
| 240 | TRBV20-1 | SARGAGDSPLH | TRBJ1-6 |
| 241 | TRBV20-1 | SARRQADQPQH | TRBJ1-5 |
| 242 | TRBV20-1 | SARVWNTEAF | TRBJ1-1 |
| 243 | TRBV20-1 | SASAGTFTDTQY | TRBJ2-3 |
| 244 | TRBV20-1 | SASPGEEKLF | TRBJ1-4 |
| 245 | TRBV20-1 | SASRQVNTEAF | TRBJ1-1 |
| 246 | TRBV20-1 | SATLQGDYGYT | TRBJ1-2 |
| 247 | TRBV20-1 | SLFGGGSTDTQY | TRBJ2-3 |
| 248 | TRBV24-1 | ATSDFQGNYGYT | TRBJ1-2 |
| 249 | TRBV24-1 | ATSDSQGLYGYT | TRBJ1-2 |
| 250 | TRBV28 | ASSRLQDHEQY | TRBJ2-7 |
| 251 | TRBV29-1 | SAGQGETQY | TRBJ2-5 |
| 252 | TRBV29-1 | SGFLGETQY | TRBJ2-5 |
| 253 | TRBV29-1 | SGGQGETQY | TRBJ2-5 |
| 254 | TRBV29-1 | SGGQGGTGELF | TRBJ2-2 |
| 255 | TRBV29-1 | SVAESSNSPLH | TRBJ1-6 |
| 256 | TRBV29-1 | SVATGWETQY | TRBJ2-5 |
| 257 | TRBV29-1 | SVDKGGDTDTQY | TRBJ2-3 |
| 258 | TRBV29-1 | SVEDQSGEKLF | TRBJ1-4 |
| 259 | TRBV29-1 | SVGAGGSGELF | TRBJ2-2 |
| 260 | TRBV29-1 | SVGAGGTGELF | TRBJ2-2 |
| 261 | TRBV29-1 | SVGAVSTDTQY | TRBJ2-3 |
| 262 | TRBV29-1 | SVGGSGANVLT | TRBJ2-6 |
| 263 | TRBV29-1 | SVGLVSTDTQY | TRBJ2-3 |
| 264 | TRBV29-1 | SVGQGGTGELF | TRBJ2-2 |
| 265 | TRBV29-1 | SVGQVSTDTQY | TRBJ2-3 |
| 266 | TRBV29-1 | SVGTVSTDTQY | TRBJ2-3 |
| 267 | TRBV30 | AWSAQGWDTGELF | TRBJ2-2 |
| 268 | TRBV30 | AWSPTGWDTGELF | TRBJ2-2 |
| 269 | TRBV30 | AWSVQGWDTDTQY | TRBJ2-3 |
| 270 | TRBV30 | AWSVTGWDTGELF | TRBJ2-2 |
| 271 | TRBV4-1 | ASSLSDSDQPQH | TRBJ1-5 |
| 272 | TRBV4-2 | ASSPGPSLGYT | TRBJ1-2 |
| 273 | TRBV4-2 | ASSPRALMNTEAF | TRBJ1-1 |
| 274 | TRBV4-2 | ASSQGLAGREETQY | TRBJ2-5 |
| 275 | TRBV4-2 | ASSQGLAGRQETQY | TRBJ2-5 |
| 276 | TRBV4-2 | ASSQGSGGNEQF | TRBJ2-1 |
| 277 | TRBV4-2 | ASSQRQGGNTIY | TRBJ1-3 |
| 278 | TRBV4-2 | ASSQVAGGEQY | TRBJ2-7 |
| 279 | TRBV4-2 | ASSRGQGATEAF | TRBJ1-1 |
| 280 | TRBV4-2 | ASSRGQGSTEAF | TRBJ1-1 |
| 281 | TRBV4-2 | ASSRLGTSTDTQY | TRBJ2-3 |
| 282 | TRBV4-2 | ASSRTLYQETQY | TRBJ2-5 |
| 283 | TRBV5-1 | ASSFDAETQY | TRBJ2-5 |
| 284 | TRBV5-1 | ASSFEETQY | TRBJ2-5 |
| 285 | TRBV5-1 | ASSFGAGEGDTQY | TRBJ2-3 |
| 286 | TRBV5-1 | ASSFGGGAGDTQY | TRBJ2-3 |
| 287 | TRBV5-1 | ASSFGGPNTGELF | TRBJ2-2 |
| 288 | TRBV5-1 | ASSFGQPSTDTQY | TRBJ2-3 |
| 289 | TRBV5-1 | ASSLGAGGQETQY | TRBJ2-5 |
| 290 | TRBV5-1 | ASSLGGGAGDTQY | TRBJ2-3 |
| 291 | TRBV5-1 | ASSLGGPNTGELF | TRBJ2-2 |
| 292 | TRBV5-1 | ASSLGIALSSYNEQF | TRBJ2-1 |
| 293 | TRBV5-1 | ASSLGSFSYEQY | TRBJ2-7 |
| 294 | TRBV5-1 | ASSLGVALSSYNEQF | TRBJ2-1 |
| 295 | TRBV5-1 | ASSLSGPNTDTQY | TRBJ2-3 |
| 296 | TRBV5-1 | ASSLVAWDTEAF | TRBJ1-1 |
| 297 | TRBV5-1 | ASSWGMNTEAF | TRBJ1-1 |
| 298 | TRBV5-5 | ASSHRTEYSGNTIY | TRBJ1-3 |
| 299 | TRBV5-5 | ASSLAQGGDTQY | TRBJ2-3 |
| 300 | TRBV5-5 | ASSFGPSNQPQH | TRBJ1-5 |
| 301 | TRBV5-5 | ASSFGVTGELF | TRBJ2-2 |
| 302 | TRBV5-5 | ASSFSVTGELF | TRBJ2-2 |
| 303 | TRBV5-5 | ASSFTNTGELF | TRBJ2-2 |
| 304 | TRBV5-5 | ASSLGRSYGYT | TRBJ1-2 |
| 305 | TRBV5-5 | ASSLKEGYGYT | TRBJ1-2 |
| 306 | TRBV5-5 | ASSLRQLYEQY | TRBJ2-7 |
| 307 | TRBV5-5 | ASSLSGLTEAF | TRBJ1-1 |
| 308 | TRBV5-5 | ASSLVNMNTEAF | TRBJ1-1 |
| 309 | TRBV5-5 | ASSRRQGYGYT | TRBJ1-2 |
| 310 | TRBV5-5 | ASSLRQEYSGNTIY | TRBJ1-3 |
| 311 | TRBV6-2 | ASSTLQGRNGYT | TRBJ1-2 |
| 312 | TRBV6-5 | ASSGRTGRYTEAF | TRBJ1-1 |
| 313 | TRBV7-2 | ASSIRAGGADTQY | TRBJ2-3 |
| 314 | TRBV7-2 | ASSIRTGDGNTQY | TRBJ2-3 |
| 315 | TRBV7-2 | ASSIRTSGSHEQY | TRBJ2-7 |
| 316 | TRBV7-2 | ASSLAFLAGEETQY | TRBJ2-5 |
| 317 | TRBV7-2 | ASSLAPRTDTQY | TRBJ2-3 |
| 318 | TRBV7-2 | ASSLRAGGADTQY | TRBJ2-3 |
| 319 | TRBV7-2 | ASSLRAGGGDTQY | TRBJ2-3 |
| 320 | TRBV7-2 | ASSLRALDLGEQY | TRBJ2-7 |
| 321 | TRBV7-2 | ASSLRASGSHEQF | TRBJ2-1 |
| 322 | TRBV7-2 | ASSLRGWETQY | TRBJ2-5 |
| 323 | TRBV7-2 | ASSLRTSGGHEQF | TRBJ2-1 |
| 324 | TRBV7-2 | ASSLRVGDTQY | TRBJ2-3 |
| 325 | TRBV7-2 | ASSLRWGGADTQY | TRBJ2-3 |
| 326 | TRBV7-2 | ASSLVPWETQY | TRBJ2-5 |
| 327 | TRBV7-2 | ASSVRTGDTQY | TRBJ2-3 |
| 328 | TRBV7-3 | ASSPGQGGDNEQF | TRBJ2-1 |
| 329 | TRBV7-3 | ASSPLGGGQDNEQF | TRBJ2-1 |
| 330 | TRBV7-3 | ASSQGQDTEAF | TRBJ1-1 |
| 331 | TRBV7-6 | ASSFGSYNEQF | TRBJ2-1 |
| 332 | TRBV7-6 | ASSLAAAGGTDTQY | TRBJ2-3 |
| 333 | TRBV7-6 | ASSLAGFDSPLH | TRBJ1-6 |
| 334 | TRBV7-6 | ASSLAGWDTEAF | TRBJ1-1 |
| 335 | TRBV7-6 | ASSLETGTTYSNQPQH | TRBJ1-5 |
| 336 | TRBV7-6 | ASSLGTVVDTGELF | TRBJ2-2 |
| 337 | TRBV7-6 | ASSVLAGAGGDTQY | TRBJ2-3 |
| 338 | TRBV7-6 | ASSWLAGTDTQY | TRBJ2-3 |
| 339 | TRBV7-6 | ASSYGSYNEQF | TRBJ2-1 |
| 340 | TRBV7-7 | ASSFLAGSDTQY | TRBJ2-3 |
| 341 | TRBV7-7 | ASSLLAGGDTQY | TRBJ2-3 |
| 342 | TRBV7-8 | ASSFDSNSPLH | TRBJ1-6 |
| 343 | TRBV7-8 | ASSLTQGAGYT | TRBJ1-2 |
| 344 | TRBV9 | ASSLGGGAGDTQY | TRBJ2-3 |
| 345 | TRBV9 | ASSNILAGEETQY | TRBJ2-5 |
| 346 | TRBV9 | ASSVGGGAGDTQY | TRBJ2-3 |
| 347 | TRBV9 | ASSVGGVYNEQF | TRBJ2-1 |
| 348 | TRAV1-1 | AVTAGSNYQLI | TRAJ33 |
| 349 | TRAV1-2 | AVLTDSWGKLQ | TRAJ24 |
| 350 | TRAV8-4 | ASLSNFGNEKLT | TRAJ48 |
| 351 | TRAV8-4 | AVSEWAGNQFY | TRAJ49 |
| 352 | TRBV12-4 | ASRLTLGTDTQY | TRBJ2-3 |
| 353 | TRBV12-4 | ASRPRGAPSYEQY | TRBJ2-7 |
| 354 | TRBV12-4 | ASSWTSWDTQY | TRBJ2-3 |
| 355 | TRBV4-3 | ASSPGPSLGYT | TRBJ1-2 |
| 356 | TRBV4-3 | ASSPRALMNTEAF | TRBJ1-1 |
| 357 | TRBV4-3 | ASSQGLAGREETQY | TRBJ2-5 |
| 358 | TRBV4-3 | ASSQGLAGRQETQY | TRBJ2-5 |
| 359 | TRBV4-3 | ASSQGSGGNEQF | TRBJ2-1 |
| 360 | TRBV4-3 | ASSQRQGGNTIY | TRBJ1-3 |
| 361 | TRBV4-3 | ASSQVAGGEQY | TRBJ2-7 |
| 362 | TRBV4-3 | ASSRGQGATEAF | TRBJ1-1 |
| 363 | TRBV4-3 | ASSRGQGSTEAF | TRBJ1-1 |
| 364 | TRBV4-3 | ASSRLGTSTDTQY | TRBJ2-3 |
| 365 | TRBV4-3 | ASSRTLYQETQY | TRBJ2-5 |
| 366 | TRBV5-6 | ASSFGPSNQPQH | TRBJ1-5 |
| 367 | TRBV5-6 | ASSFGVTGELF | TRBJ2-2 |
| 368 | TRBV5-6 | ASSFSVTGELF | TRBJ2-2 |
| 369 | TRBV5-6 | ASSFTNTGELF | TRBJ2-2 |
| 370 | TRBV5-6 | ASSLGRSYGYT | TRBJ1-2 |
| 371 | TRBV5-6 | ASSLKEGYGYT | TRBJ1-2 |
| 372 | TRBV5-6 | ASSLRQLYEQY | TRBJ2-7 |
| 373 | TRBV5-6 | ASSLSGLTEAF | TRBJ1-1 |
| 374 | TRBV5-6 | ASSLVNMNTEAF | TRBJ1-1 |
| 375 | TRBV5-6 | ASSRRQGYGYT | TRBJ1-2 |
| 376 | TRBV5-6 | ASSLRQEYSGNTIY | TRBJ1-3 |
| 377 | TRBV6-3 | ASSTLQGRNGYT | TRBJ1-2 |

TABLE 3
Newly-identified CD-associated
TCRα and TCRβ chain consensus sequences:

| SEQ | | | |
|---|---|---|---|
| ID NO | V-Gene | Consensus CDR3 Sequence | J-Gene |
| 378 | TRBV24-1 | ATSD(F/S)QG(L/N)YGYT | TRBJ1-2 |
| 379 | TRBV29-1 | SxG(A/Q)GG(S/T)GELF | TRBJ2-2 |
| 380 | TRBV29-1 | SVGxVSTDTQY | TRBJ2-3 |
| 381 | TRBV29-1 | S(A/G)(F/G)(L/Q)GETQY | TRBJ2-5 |
| 382 | TRBV30 | AWSx(Q/T)GWDTGELF | TRBJ2-2 |
| 383 | TRBV4-2 | ASSRGQG(A/S)TEAF | TRBJ1-1 |
| 384 | TRBV4-2 | ASSQGLAGR(E/Q)ETQY | TRBJ2-5 |
| 385 | TRBV5-1 | ASSLG(I/V)ALSSYNEQF | TRBJ2-1 |
| 386 | TRBV5-1 | ASS(F/L)GGPNTGELF | TRBJ2-2 |
| 387 | TRBV5-1 | ASS(F/L)(S/G)x(P/G)x(T/G)DTQY | TRBJ2-3 |
| 388 | TRBV5-1 | ASSFD(A/G)ETQY | TRBJ2-5 |
| 389 | TRBV5-5 | ASS(L/R)xx(S/G)YGYT | TRBJ1-2 |
| 390 | TRBV5-5 | ASSFx(V/N)TGELF | TRBJ2-2 |
| 391 | TRBV7-2 | ASSLR(A/T)SG(G/S)HEQF | TRBJ2-1 |
| 392 | TRBV7-2 | ASS(I/L)RxG(G/D)(A/G)(N/D)TQY | TRBJ2-3 |
| 393 | TRBV7-2 | ASS(LN)R(T/V)GDTQY | TRBJ2-3 |
| 394 | TRBV7-2 | ASSL(R/V)(P/G)WETQY | TRBJ2-5 |
| 395 | TRBV7-6 | ASS(FN)GSYNEQF | TRBJ2-1 |
| 396 | TRBV7-6 | ASS(LN)(L/A)(A/S)(A/G)(A/G) | TRBJ2-3 |
| | | G(T/G)DTQY | |
| 397 | TRBV7-6 | ASSxLAGxDTQY | TRBJ2-3 |
| 398 | TRBV9 | ASS(L/V)GGGAGDTQY | TRBJ2-3 |
| 399 | TRAV1-2 | AVTS(S/T)NTGKLI | TRAJ37 |
| 400 | TRAV1-2 | AVRAVxSGGYNKLI | TRAJ4 |
| 401 | TRAV12-1 | VVNx(A/Y)SSASKII | TRAJ3 |
| 402 | TRAV12-2 | AV(P/S)NRDDKII | TRAJ30 |
| 403 | TRAV12-3 | AMx(E/Q)AAGNKLT | TRAJ17 |
| 404 | TRAV12-3 | AM(K/R)DYGQNFV | TRAJ26 |
| 405 | TRAV12-3 | AMS(A/E)GTGNQFY | TRAJ49 |
| 406 | TRAV12-3 | AMS(D/E)(I/L)PGGSNYKLT | TRAJ53 |
| 407 | TRAV12-3 | AM(N/T)DYGNNRLA | TRAJ7 |
| 408 | TRAV13-2 | AE(G/T)(N/D)AGGTSYGKLT | TRAJ52 |
| 409 | TRAV19 | ALSEG(G/S)NAGNMLT | TRAJ39 |
| 410 | TRAV19 | ALS(E/G)(G/A)GANSKLT | TRAJ56 |
| 411 | TRAV22 | AVE(L/R)QGAQKLV | TRAJ54 |
| 412 | TRAV26-1 | lx(F/Y)NDYKLS | TRAJ20 |
| 413 | TRAV26-1 | IVxNARLM | TRAJ31 |
| 414 | TRAV26-1 | l(G/P)NYGGSQGNLI | TRAJ42 |
| 415 | TRAV26-1 | IV(F/Y)GGSQGNLI | TRAJ42 |
| 416 | TRAV35 | A(A/G)NDYKLS | TRAJ20 |
| 417 | TRAV35 | AG(N/Q)(L/Y)GGATNKLI | TRAJ32 |
| 418 | TRAV35 | AG(F/H)NTDKLI | TRAJ34 |
| 419 | TRAV35 | AGQ(P/V)GSSNTGKLI | TRAJ37 |
| 420 | TRAV4 | LVG(D/G)xGGYNKLI | TRAJ4 |
| 421 | TRAV4 | LVGD(D/P)TGFQKLV | TRAJ8 |
| 422 | TRAV41 | AVExGSNYQLI | TRAJ33 |
| 423 | TRAV8-3 | AV(A/G)xDRGSTLGRLY | TRAJ18 |
| 424 | TRAV8-3 | AVGx(A/S)EYGNKLV | TRAJ47 |
| 425 | TRAV9-2 | AL(A/S)E(Y/G)NFNKFY | TRAJ21 |
| 426 | TRAV9-2 | ALSD(P/Q)TGTASKLT | TRAJ44 |
| 427 | TRBV18 | ASS(L/P)AGWDTEAF | TRBJ1-1 |
| 428 | TRBV19 | ASS(I/T)GTSGETQY | TRBJ2-5 |
| 429 | TRBV4-3 | ASSRGQG(A/S)TEAF | TRBJ1-1 |
| 430 | TRBV4-3 | ASSQGLAGR(E/Q)ETQY | TRBJ2-5 |
| 431 | TRBV5-6 | ASS(L/R)xx(S/G)YGYT | TRBJ1-2 |
| 432 | TRBV5-6 | ASSFx(V/N)TGELF | TRBJ2-2 |

x indicates any amino acid residue.

As used herein, amino acid sequences are represented by the conventional one-letter code.

As used herein, CD4+ cells are lymphocytes expressing CD4 in the cell membrane, i.e. that they are positive in assays relying on anti-CD4 antibodies. The skilled person can easily identify and isolate CD4+ T-cells from a cell population using e.g. fluorescence-activated cell sorting (FACS).

As used herein, effector memory T-cells (TEM cells), are T-cells that have clonally expanded and differentiated into effector T-cells as a result of stimulation by their cognate antigens. These TEM lymphocytes express CD45RO, but lack expression of CCR7, CD45RA and L-selectin (also known as CD62L). Such cells may have intermediate to high expression of CD44 and they may lack lymph node-homing receptors. The skilled person can easily identify and isolate effector memory T-cells from a cell population using e.g. FACS.

As used herein, the normalised number of cells, means a relative fraction of cells in a sample. A normalised number of cells may be expressed e.g. as cells per thousand, cells per million, etc.

Gluten-specific TCR sequences may be clonally expanded as a result of gluten stimulation in celiac disease patients. By normalising the count of T-cells expressing such TCRs, an increase or decrease in the proportion of gluten-specific T-cells in a patient may be identified. An identifiable increase in the proportion gluten-specific T-cells in a CD patient generally occurs following gluten challenge. Herein, the inventors have measured the number of clonotypes in a sample, as estimated using the MiXCR software, expressing a TCRα sequence and/or a TCRβ sequence selected from Table 1 and/or from Table 2.

Methods are disclosed herein for diagnosing celiac disease in a human subject (and optionally also treating celiac disease in the same subject). Also disclosed herein are methods for detecting TCR sequences in T-cells in a sample from a human subject. Such a human subject may be of any age, e.g. a child or an adult, and may be male or female. The subject preferably is suspected of having celiac disease based on their clinical history. Methods are also disclosed for monitoring the response of a human subject to treatment for celiac disease. Similarly, such a human subject may be of any age, e.g. a child or an adult, and may be male or female. In this instance, the human subject has previously been diagnosed with celiac disease and is undergoing treatment for the condition, e.g. the subject may be on a gluten-free diet.

The methods may be performed wholly in vitro, using a sample already provided by a human subject. However, in an embodiment, the method may comprise a step of obtaining a sample from a human subject. The sample may be obtained from any human subject. The human subject may be of any age, e.g. a child or an adult, and may be male or female. The subject may be suspected of having celiac disease, but equally may be a healthy subject, e.g. a volunteer.

The first step of the method may be the obtaining of a sample comprising T-cells from a human subject. This may be any cellular (i.e. cell-containing) sample, which contains T-cells. Any tissue which comprises T-cells may be used, e.g. blood, lymph, etc. The sample may be of a liquid tissue or a solid tissue. A solid tissue may be e.g. a biopsy sample, that is to say a tissue sample removed from the body for examination. If the sample is a solid tissue it is preferably a sample of the wall of the small intestine. Such a sample may be obtained by e.g. gastrointestinal endoscopy. Preferably the sample is of a liquid tissue which may be obtained by a non-invasive procedure. In a particular embodiment the sample is a blood sample. A blood sample may be obtained by e.g. phlebotomy. The skilled person is able to obtain a blood sample from a patient without particular instruction. The tissue sample used may comprise at least 100,000, 250,000, 500,000, 750,000, 1 million, 1.25 million, 1.5 million or 2 million T-cells. In a particular embodiment, the tissue sample comprises at least 100,000, 250,000, 500,000, 750,000, 1 million, 1.25 million, 1.5 million or 2 million CD4+ effector memory T-cells.

Nucleic acids are then isolated from the sample. In an alternative embodiment, the first step of the method is the isolation of nucleic acids from a sample obtained from the subject, wherein said sample comprises T-cells. The sample may be as described above.

If the sample is a blood sample, peripheral blood mononuclear cells (PBMCs) are preferably isolated from the whole blood for use in the method. PBMCs may be isolated from buffy coats obtained by density gradient centrifugation of whole blood, for instance centrifugation through a LYMPHOPREP™ gradient, a PERCOLL™ gradient or a FICOLL™ gradient. T-cells may be isolated from PBMCs by depletion of the monocytes and B-cells, for instance by using CD14 and CD19 DYNABEADS®. In some embodiments, red blood cells may be lysed prior to the density gradient centrifugation.

If the sample is a biopsy sample it is, as mentioned above, preferably obtained from the small intestine of the subject. The lamina propria is the most CD4+ T-cell-rich region of the human small intestine wall. In a particular embodiment, a biopsy sample obtained from the small intestine of the subject is processed to isolate lamina propria cells, which are used in the method of the invention.

The sample may be enriched for CD4+ effector memory T-cells prior to nucleic acid extraction. That is to say, the proportion of CD4+ effector memory T-cells in the sample may be increased. Enrichment may be performed by either negative selection (cells which are not CD4+ effector memory T-cells are removed from the sample) or positive selection (in which CD4+ effector memory T-cells are specifically isolated). Negative selection may be performed by removing cells expressing surface markers not present on CD4+ effector memory T-cells. As noted above, CD4+ effector memory T-cells may be characterised by their expression of CD45RO and absence of expression of CCR7, CD45RA and L-selectin. Accordingly, negative selection may be performed by the removal from the sample of cells expressing CCR7, CD45RA and/or L-selectin. Positive selection may be performed by the isolation of cells in the sample expressing CD4 and/or CD45RO. Such selection may be performed using standard methods in the art, e.g. FACS sorting or using an appropriate commercial kit (e.g. the human CD4+ Effector Memory T Cell negative Isolation kit provided by Miltenyi).

It has been found that immune sensitivity to gluten may in particular be determined by measurement of the number of T-cells, particularly CD4+ effector memory T-cells, in a sample expressing the gluten-specific TCR sequences set forth in Table 1 and Table 2. As disclosed herein, a determination may be made of the number, or more particularly the frequency, of nucleotide sequences encoding the TCR sequences set forth in Table 1 and Table 2 within the sample. This can be used directly. Thus, the number or frequency of the nucleotide sequences can be taken as being an indicator for, or representative for, or a proxy for, the number of T-cells. Thus, an actual value for the number of cells does not need to be determined as such, although in an embodiment it could be. The number of nucleotide sequences (i.e. the abundance) in the sample can be determined (e.g. a count, or number of “reads” from the sequencing step) and this may be used to determine a score which represents a clonotype count, that is a count of each particular clonotype determined. A clonotype here may be taken as referring to a particular TCRα or TCRβ, and not necessarily paired TCRα and TCRβ sequences.

After enrichment, the sample may comprise at least 70%, 80%, 90%, 95% or 99% CD4+ effector memory T-cells. The percentage of CD4+ effector memory T-cells in the sample is preferably the percentage of the total number of cells in the sample which are CD4+ effector memory T-cells.

Nucleic acids may be isolated from the sample using any method known in the art. In a particular embodiment of the invention, the nucleic acid isolated from the sample is genomic DNA (gDNA). In another embodiment of the invention, the nucleic acid isolated from the sample is RNA, preferably mRNA. The skilled person is able to isolate nucleic acids (including gDNA and/or RNA) from a tissue sample without particular instruction. Suitable methods include the phenol/chloroform technique and the use of an appropriate commercial kit, e.g. the DNeasy Blood and Tissue Kit (Qiagen, Germany) or the FastRNA Pro Blue kit (MP Biomedicals, USA).

Nucleic acids may be isolated in bulk or from single cells. If nucleic acids are isolated in bulk, the nucleic acids are isolated from all cells in the tissue sample together, and the resultant isolated nucleic acids are a mixture of the nucleic acids isolated from all cells in the tissue sample. If nucleic acids are isolated from single cells, the tissue sample is sorted into single cells (e.g. by FACS sorting on an Aria-II or similar flow sorting apparatus) and nucleic acids from each single cell separately isolated and analysed. Bulk nucleic acid isolation allows the analysis of general population characteristics, while separate isolation of DNA from individual cells allows the analysis of the general population at cellular level. Isolation of nucleic acids and sequencing of nucleic acids on a single cell level may readily permit the number, or frequency, of T-cells expressing the TCR sequences to be determined.

Once the nucleic acids have been isolated, sequencing is performed. If gDNA was isolated in the nucleic acid isolation step, the sequencing may be performed directly on the isolated gDNA (or as described below, the gDNA may first be subjected to an amplification step, and amplification products can be subjected to sequencing). If RNA (for instance mRNA) was isolated from the subject in the nucleic acid isolation step, the RNA is preferably reverse transcribed into cDNA, and the sequencing performed on the cDNA (or an amplification product thereof). The skilled person is able to perform reverse transcription of RNA without particular instruction using standard methods in the art. Reverse transcription may in particular be performed using a suitable commercial kit of which numerous are available, e.g. the RETROscript Reverse Transcription kit or the Superscript IV First-Strand Synthesis System (both Thermo Fisher Scientific, USA). Accordingly, the method may further comprise a step of performing a reverse transcription reaction, e.g. using a template switch oligo together with the cellular-derived RNA, to generate cDNA. The isolated RNA may be isolated mRNA. The synthesised cDNA may then be sequenced.

As noted above, the sequencing may be performed directly on the nucleic acids isolated from the tissue sample. In preferred embodiments, however, nucleotide sequences encoding TCR chains are amplified prior to sequencing. Thus the method may further comprise a step of amplifying nucleotide sequences which encode TCRα chains and TCRβ chains. Such amplification may be performed by any known DNA amplification method, preferably by PCR.

If amplification is performed, nucleotide sequences which encode all the TCRα and TCRβ chains in the sample may be amplified (e.g. all nucleotide sequences in the sample which encode a TCRα or TCRβ chain may be amplified). In another embodiment only nucleotide sequences which encode TCRβ chains are amplified (i.e. nucleotide sequences which encode TCRα chains are not amplified). Methods for performing such amplification are known in the art. Amplification may be performed using a mix of primers which comprises primers which bind every V gene segment and every J gene segment so that each TCR chain may be specifically amplified. Alternatively, primers which bind the V-gene segment may be replaced by one or more primers which specifically hybridise to cDNA upstream of the V gene segment and/or primers which bind the J gene segment may be replaced by primers which bind the constant region gene segment. In an embodiment in which a template switch method is used in the reverse transcription step, one or more primers may be used which specifically hybridise to the cDNA sequence introduced by the template switch oligo upstream of the V gene segment. Amplification of nucleotide sequences encoding TCRα and TCRβ chains yields a library of amplification products which may be sequenced. The primers which bind the V gene segment (or cDNA upstream thereof) are designed such that they may be used in combination with the primers which bind the J gene segment (or TCR constant region gene segment) to obtain an amplification product.

In another embodiment, nucleotide sequences which encode TCRα chains and TCRβ chains (or alternatively, just nucleotide sequences which encode TCRβ chains) are amplified using primers which bind only the V gene segments and J gene segments included in Tables 1 and 2 herein. In this embodiment, the amplification may be performed using a composition suitable for multiplex PCR and comprising a plurality of nucleic acid primers wherein the composition comprises primers able to specifically hybridise to the TCR V-gene segments specified in Table 1 and Table 2 and primers able to specifically hybridize to the TCR J-gene segments specified in Table 1 and Table 2, wherein an amplification product may be obtained using a combination of a primer able to specifically hybridise to a TCR V-gene segment and a primer able to specifically hybridise to a TCR J-gene segment.

In another embodiment, nucleotide sequences which encode TCRα chains and TCRβ chains (or alternatively, just nucleotide sequences which encode TCRβ chains) are amplified using primers which bind only the V gene segments included in Tables 1 and 2 herein and primers which bind TCR constant region gene segments. In this embodiment, the amplification may be performed using a composition suitable for multiplex PCR and comprising a plurality of nucleic acid primers wherein the composition comprises primers able to specifically hybridize to the TCR V-gene segments specified in Table 1 and Table 2 and primers able to specifically hybridise to a nucleotide sequence encoding a TCR constant region, wherein an amplification product may be obtained using a combination of a primer able to specifically hybridise to a TCR V-gene segment and a primer able to specifically hybridise to a nucleotide sequence encoding a TCR constant region.

Alternatively, amplification may be performed such that only nucleotide sequences which encode TCRα and/or TCRβ chains of interest are amplified. By TCRα and/or TCRβ chains of interest is meant the at least two TCRα and/or TCRβ chains whose abundance contributes to the score of the TCR dataset. In this embodiment, the amplification is performed using only primers which bind the V gene segments of the TCRα/TCRβ chains of interest and primers which bind the J gene segments of the TCRα/TCRβ chains of interest.

Amplification must be performed so that the amplification product contains sufficient sequence information to allow the V gene segment and the J gene segment of the TCR chain to be identified, and the CDR3 sequence to be determined. The primers may bind at or beyond the ends of the V and C gene segments (i.e. primers may be used which bind DNA upstream of the V gene segment and within the TCR constant region gene segment, or a primer which binds the 5′ end of the V gene segment and a primer which binds the 3′ end of the J gene segment may be used), to enable the amplification of at least the entire nucleotide sequence which encodes the variable region of the TCR chain. Alternatively, the primers may bind within the V gene and J gene segments, so that not all of the nucleotide sequence encoding the TCR chain variable region is amplified (i.e. only a part of the nucleotide sequence encoding the TCR chain variable region is amplified). If only a part of the nucleotide sequence encoding the TCR chain variable region is amplified, the part must be sufficient that the V and J gene segments which form the variable region can be identified based on their sequence, and the CDR3 sequence can be determined.

Accordingly, the method of the invention may comprise a step wherein nucleotide sequences which encode all or part of TCRα chains and TCRβ chains are amplified (or alternatively, just nucleotide sequences which encode all or part of TCRβ chains). Step (b) (or in certain aspects step (c)) may thus alternatively be more particularly defined as a step of sequencing nucleotide sequences of, or obtained or derived from, the nucleic acids (i.e. the isolated nucleic acids) which encode all or part of TCRα chains and/or TCRβ chains to provide a TCR dataset. If nucleotide sequences encoding only a part of TCRα chains and/or TCRβ chains are amplified, the part of each TCR chain amplified preferably comprises the entirety of the nucleotide sequence encoding the variable region of the TCR chain. At minimum, the part of each TCR chain amplified comprises sufficient sequence information to allow the V and J gene segments which form the variable region to be identified, and the CDR3 sequence to be determined.

Nucleic acid sequencing may be performed using any method known to the skilled person, e.g. Sanger sequencing. Preferably, the sequencing is performed using a high-throughput sequencing method, utilising e.g. an Illumina platform (such as a HiSeq or MiSeq platform, obtainable from Illumina, USA) or a nanopre sequencing platform (e.g. the MinION device, GridION device or PromethION device, available from Oxford Nanopore Technologies, UK).

The nucleotide sequences which are sequenced include nucleotide sequences encoding TCRα chains and TCRβ chains. In another embodiment, just nucleotide sequences which encode TCRβ chains are sequenced. All isolated nucleic acids may be sequenced, or only nucleotide sequences encoding TCR chains may be sequenced. If only nucleotide sequences encoding TCR chains are sequenced, some or all of the nucleotide sequences in the sample encoding TCR chains are sequenced. In a particular embodiment only nucleotide sequences encoding TCR chains comprising a V gene segment listed in Table 1 or 2 and a J gene segment listed in Table 1 or 2 are sequenced. In another embodiment, only nucleotide sequences encoding TCR chains comprising a V gene segment of a TCR chain of interest and J gene segment of a TCR chain of interest are sequenced. These embodiments are discussed above in the context of the generation of amplification products for use in sequencing.

The nucleotide sequences sequenced may encode all or part of TCRα and/or TCRβ chains. The nucleotide sequences sequenced preferably encode at least the entirety of the variable regions of TCRα and/or TCRβ chains, but at minimum comprises sufficient sequence information to allow the V and J gene segments which form the variable region of the encoded TCRα or TCRβ chain to be identified, and the CDR3 sequence to be determined. These embodiments are discussed above in the context of the generation of amplification products for use in sequencing.

In accordance with the nature of the amplification products which may be generated for use in sequencing, the step of sequencing nucleotide sequences which encode TCRα chains and nucleotide sequences which encode TCRβ chains should be understood to refer to a step of: sequencing nucleotide sequences which encode all or part of TCRα chains and/or nucleotide sequences which encode all or part of TCRβ chains, or their complementary sequences, wherein the nucleotide sequences sequenced preferably encode, or are complementary to sequences which encode, at least the entire variable regions of TCRα chains and/or TCRβ chains. The nucleotide sequences sequenced comprise at minimum sufficient sequence information to allow the V and J gene segments which form the variable region of the encoded TCRα or TCRβ chains to be identified, and the CDR3 sequences to be determined.

The TCR chain nucleotide sequences obtained together form a TCR dataset, that is to say a set of TCR sequence data which contains information as to the TCR chains encoded by T-cells in the tissue sample.

The TCR dataset is analysed to assign it a score. The score is determined by the abundance in the dataset of nucleotide sequences which encode at least two TCRα or TCRβ amino acid sequences, wherein said at least two TCRα or TCRβ amino acid sequences comprise:
(i) at least one TCRα or TCRβ amino acid sequence selected from SEQ ID NOs: 1 to 50; and
(ii) at least one TCRα or TCRβ amino acid sequence selected from SEQ ID NOs: 51 to 432.

By abundance is meant the number, or count, of the sequences. The abundance may be, or may be based on, the number of sequence reads obtained in the sequencing step (see further below).

If nucleotide sequences encoding only parts of TCR chains are sequenced, the presence in the dataset of a nucleotide sequence encoding a TCR chain of interest is deduced from the presence of a part of the sequence, and is regarded as if the entire nucleotide sequence encoding the TCR chain of interest is present in the dataset.

The combination of TCR chain sequences to be used in the analysis may include any TCR chain sequence selected from SEQ ID NOs: 1 to 50 and any TCR chain sequence selected from SEQ ID NOs: 51 to 432. Preferably, more than two TCR chain sequences are used for the analysis. In particular embodiments, the score is determined by the abundance in the dataset of nucleotide sequences which encode at least 50, 100, 150, 200, 250, 300, 350 or 400 TCRα and/or TCRβ amino acid sequences selected from SEQ ID NOs: 1 to 432. In other embodiments the CDR chain consensus sequences of Table 3 are not included in the analysis, and the score is determined by the abundance in the dataset of nucleotide sequences which encode at least 50, 100, 150, 200, 250, 300 or 350 TCRα and TCRβ amino acid sequences set out in SEQ ID NOs: 1 to 377. Any combination of TCRα and/or TCRβ sequences may be used to calculate the score of the dataset.

In a particular embodiment, the score is determined by the abundance in the dataset of nucleotide sequences which encode at least the 229 TCRα and TCRβ amino acid sequences set forth in SEQ ID NOs: 1, 2, 4-15, 17, 18, 20-25, 27-37, 39-48, 51, 53-55, 59, 60, 62, 64, 68, 69, 72-75, 77-79, 81-85, 87, 88, 90-92, 94, 96-105, 107, 108, 111, 112, 117-120, 122, 124, 127-129, 132, 133, 137-141, 143, 145, 151-153, 156, 157, 159, 163-165, 168-171, 173, 176-179, 182, 184, 185, 188-190, 194-196, 198, 199, 201, 202, 204-206, 209-211, 213, 214, 218-218, 220, 223-225, 228, 230, 232-234, 238, 241-250, 252, 253, 255, 258-263, 265, 266, 270, 271, 275-277, 283, 290-292, 294, 296, 297, 299-301, 303-309, 312, 314, 316, 318, 319, 322, 324, 330, 331, 333, 336, 339, 341, 342, 344, 346, 349, 350, 352, 358-360, 366, 367 and 369-375.

In a preferred embodiment, the score is determined by the abundance in the dataset of nucleotide sequences which encode the TCRα and TCRβ amino acid sequences set out in SEQ ID NOs: 1 to 377. That is to say, all 377 sequences in Tables 1 and 2 are included in the analysis.

In another embodiment, the score is determined by the abundance in the dataset of nucleotide sequences which encode the TCRα and TCRβ amino acid sequences set out in SEQ ID NOs: 1 to 432. That is to say, all 432 sequences in Tables 1, 2 and 3 are included in the analysis. In a particular embodiment the score of the dataset is calculated based on the abundance in the dataset of all TCRβ chain sequences set forth in SEQ ID NOs: 1 to 432 (i.e. the TCRα chain sequences are not included).

By the “abundance” of the nucleotide sequences of interest in the dataset is simply meant the number of times the nucleotide sequences of interest appear in the dataset. The nucleotide sequences of interest are those nucleotide sequences which encode the TCRα and TCRβ amino acid sequences which are the subject of analysis, i.e. those nucleotide sequences which contribute to the score. The abundance of the nucleotide sequences of interest corresponds to the total number of sequencing reads which comprise a sequence of interest. Thus the score itself is not normalised or adjusted to sample size or suchlike. For instance, if a dataset comprised 200 reads which comprise a nucleotide sequence of interest, the score of that dataset would be 200, regardless of any other factors. Any appropriate method may be used to calculate the score of the dataset. The score may be calculated manually, but is preferably calculated using appropriate software, e.g. the MiXCR programme (Bolotin, D. et al., Nat. Methods 12(5): 380-381, 2015, herein incorporated by reference). A programme such as MiXCR may be used to calculate an accurate estimate of the total number of clonotypes within a sample.

Once calculated, the score is normalised to provide a normalised score. The normalised score is representative of either the frequency of the nucleotide sequences of interest in the TCR dataset or the frequency of T-cells expressing the nucleotide sequences in the tissue sample. While the score initially assigned to the TCR dataset is raw and affected by factors such as sample size, the number of T-cells within the sample and sequencing depth, the normalised score is not affected by such factors and is instead an accurate measure of how common the TCR sequences of interest are in the sample, enabling valid comparisons of the frequency of the sequences of interest to be performed between samples, both in terms of comparison between samples obtained from different individuals and samples taken from the same individual at different times. The normalised score may also be compared to a defined threshold to determine whether a sample comprises more celiac disease-associated TCR sequences than would be expected in a healthy individual, which is indicative of celiac disease.

Normalisation may be performed by any suitable method known in the art. For example, normalisation may be performed by dividing the number of sequencing reads which comprise a nucleotide sequence of interest by the total number of sequencing reads, thus providing a normalised score in the form of the proportion of sequencing reads which comprise a nucleotide sequence of interest (i.e. the frequency of sequencing reads which comprise a nucleotide sequence of interest). Alternatively, normalisation may be performed by dividing the total number of sequencing reads by the number of sequencing reads which comprise a nucleotide sequence of interest. This provides a normalised score in the form of “number of total reads per read of interest”. For conciseness, a “sequencing read” may be referred to herein as simply a “read”.

Another suitable method of normalisation is dividing the estimated number of T-cell clonotypes which express a TCR sequence of interest by the estimated total number of clonotypes observed (as noted above, clonotype numbers may be calculated from the raw data using a suitable computer programme, such as MiXCR), thus determining the proportion (or frequency) of clonotypes of interest within the dataset. A clonotype of interest as defined herein is a T-cell clonotype which comprises a TCRα or TCRβ chain of interest (that is to say a TCRα chain or TCRβ chain encoded by a nucleotide sequence which contributes to the score).

If the TCR sequence data has been collected by single cell sequencing methods, normalisation may also be performed by dividing the number of T-cells expressing a TCR sequence of interest by the total number of T-cells sequenced, thus determining the proportion (or frequency) of T-cells expressing TCR sequences of interest within the sample. In other words, the normalised score may be the frequency in the sample of T-cells which express a TCRα chain or TCRβ chain encoded by a nucleotide sequence which contributes to the score. Such a normalised score may be presented in the form T-cells per thousand, T-cells per million, or suchlike.

Using the methods detailed above, normalisation of the score based on the frequency of sequencing reads which comprise a nucleotide sequence of interest or the frequency of clonotypes of interest within the dataset provides a normalised score representative of the frequency of the nucleotide sequences in the TCR dataset. Any other suitable method of normalisation which provides a normalised score as defined herein and known to the skilled person may alternatively be used.

In a particular embodiment, the normalised score is the frequency in the TCR dataset of sequencing reads which comprise a nucleotide sequence of interest, that is to say the frequency in the TCR dataset of nucleotide sequences which contribute to the score. Such a normalised score may be presented in the form of nucleotide sequences which contribute to the score per thousand reads, or nucleotide sequences which contribute to the score per million reads, or suchlike.

The normalised score is compared to a defined threshold. The defined threshold is defined using the same units as the normalised score (e.g. nucleotide sequences which contribute to the score per million reads). If the method is performed for the purpose of diagnosing celiac disease in a subject, the defined threshold is generally the diagnosis threshold. If the normalised score of a subject is equal to or exceeds the diagnosis threshold, the subject may be diagnosed as having celiac disease; if the normalised score of a subject is less than the diagnosis threshold, celiac disease may be excluded from the diagnosis for the subject's symptoms.

In particular embodiments, the defined threshold is or is at least 240, 270, 300, 350, 400, 450 or 500 nucleotide sequences which contribute to the score per million reads. If the method is performed for the purposes of diagnosing celiac disease in a subject, the subject may thus be considered likely to be suffering from celiac disease, or diagnosed with celiac disease, if their normalised score is at least 240, 270, 300, 350, 400, 450 or 500 nucleotide sequences which contribute to the score per million reads.

As noted above, if a subject has a normalised score which is less than defined threshold, celiac disease may be excluded from the diagnosis for that subject's symptoms, or the subject may be considered very unlikely to be suffering from celiac disease. In particular embodiments, celiac disease may be excluded from a subject's diagnosis if their normalised score is less than 500, 450, 400, 350, 300, 270, 240, 230, 200 or 180 nucleotide sequences which contribute to the score per million reads.

The method is particularly robust for exclusion of celiac disease from a subject's diagnosis when combined with a negative test result for HLA-DQ2 and/or HLA-DQ8. The term HLA-DQ2 refers in particular to HLA-DQ2.2 and HLA-DQ2.5. In particular, if a subject is HLA-DQ2 negative and HLA-DQ8 negative, and has a normalised score less than the defined threshold, celiac disease may be excluded from the diagnosis of that subject's symptoms. The defined threshold may be as described above.

If the method is performed in order to monitor the response of a subject to treatment for celiac disease, comparison of their normalised score to the defined threshold may be used to determine the response of the subject to treatment. In this instance, the defined threshold may be the normalised score of the subject prior to the initiation of treatment, in which case a normalised score lower than the defined threshold generally indicates that the treatment is effective and reducing the number of gluten-specific T-cells active in the subject, and conversely a normalised score higher than the defined threshold may indicate that the condition is refractory to treatment, or that the subject has not been keeping to their treatment regime (e.g. has not properly implemented a gluten-free diet). Alternatively, if the method is performed in order to monitor the response of a subject to treatment for celiac disease, the defined threshold may be the normalised score of the subject on the previous occasion the test was performed, allowing the continuous monitoring of the efficacy of their treatment regime.

If the calculation of a normalised score of a subject is performed as part of a method for diagnosis and treatment of celiac disease, if the subject is diagnosed with celiac disease as described above, treatment for celiac disease is then administered to the subject. The treatment for celiac disease may in particular be the prescription of a gluten-free diet.

Alternatively, the treatment for celiac disease may be the targeting of gluten-specific T-cells (in particular T-cells which express a TCR chain of any one of SEQ ID NOs: 1-432 or 1-377) with epitope-specific immunotherapy, in order to deplete or eradicate these cells from the subject. This approach is currently being explored in the clinic (Goel, G. et al., Lancet Gastroenterol. Hepatol. 2(7):479-493, 2017, herein incorporated by reference). In another embodiment the treatment may comprise depleting or eliminating activated T-cells after oral gluten challenge in CD patients in remission.

### Examples

### Methods

### Human Material

All patients donated up to 100 ml of blood and 6-12 duodenal biopsies. In addition, we had access to cryopreserved PBMCs or T-cell lines derived from single duodenal biopsies donated in 1988-2000 of five subjects. In the gluten challenge study, treated CD patients on GFD were recruited to a 14-day gluten challenge clinical study. We obtained 50-100 ml of citrated blood at baseline, day 6 and day 14 as well as eight duodenal biopsies at baseline and on day 14. In one case (CD1300), we also obtained a blood sample on day 28.

### Tetramer Staining and Cell Sorting

Samples from HLA-DQ2.5+ subjects were stained with a mix of four PE-conjugated HLADQ2.5:gluten tetramers representing gluten T-cell epitopes; DQ2.5-glia-α1a, DQ2.5-glia-α2, DQ2.5-glia-ω1 and DQ2.5-glia-ω2. Samples from one HLA-DQ8+ subject (CD1374) were stained with a mix of HLA-DQ:DQ8-glia-α1 and HLA-DQ8:DQ8-glia-γ1b tetramers. Single cell suspensions of duodenal biopsies were directly stained with surface antibody mix and LIVE/DEAD marker after tetramer staining. Tetramer-stained PBMC samples were enriched as described by Christophersen et al. United European Gastroenterol J. 2014; 2(4):268-278. We sorted HLA-DQ:gluten tetramer+CD4+ effector-memory gut-homing (CD62L− CD45RA− integrin-β7+) T-cells in blood and tetramer+CD4+ T-cells in biopsies on an Aria-II cell sorter (BD Biosciences).

### TCR Sequencing

### Single-Cell TCR Sequencing Using Multiplex PCR

To obtain paired TCRα and TCRβ sequences, we performed PCR with multiplexed primers covering all TCRα and TCRβ V genes according to the published protocol (Han A. et al., Nat Biotechnol. 32(7):684-692, 2014, herein incorporated by reference). However, our method differed to the published protocol in that, we performed cDNA synthesis and the first PCR reaction in two separate steps. We sorted single cells into 96-well plates containing 5 μl capture buffer (20 mM Tris-HCl pH 8, 1% NP-40, 1 U/μl RNase Inhibitor (optional)). The plates were stored at −70° C. until cDNA synthesis to facilitate cell lysis. For cDNA synthesis, we added 5 μl cDNA mix (1×FS buffer, 1 mM dNTP, 2.5 mM DDT, 1 μM oligo d(T) (5′-CTGAATTCT(16)-3′), 1 μM reverse TRAC (5′-AGTCAGATTTGTTGCTCCAGGCC-3′) and TRBC (5′-TTCACCCACCAGCTCAGCTCC-3′) primers, 1.5 U/μl RNase Inhibitor, 2.5 U/μl Superscript II in final 10 μl reaction volume). The cDNA synthesis was carried out at 42° C. for 50 min followed by an inactivation step at 72° C. for 10 min. The cDNA plates were stored at −20° C. Each of the three nested PCR steps was carried out in a total volume of 10 μl using 1 μl cDNA/PCR template and KAPA HiFi HotStart ReadyMix (Kapa Biosystems). For the two first nested PCR reactions, the final concentration of each TCR V-gene and C-gene primer was 0.06 μM and 0.3 μM, respectively. In the final barcoding PCR step, we added 5′-barcoding primers (0.044 μM) and 1:4 ratio of the 3′-barcoding primers, TRBC (0.044 μM) and TRAC (0.18 μM). In addition, Illumina Paired-End primers were added to the master mix (0.5 μM each). Primer sequences and cycling conditions for all three PCR reactions are provided in the original protocol (Han et al., supra).

Bulk TCR Sequencing by PCR Amplification of Template-Switched cDNA

When feasible due to high cell numbers, we sorted in bulk 150-3000 T cells in an Eppendorf tube containing 50-100 μl TCL lysis buffer (Qiagen) supplemented with 1% 3-mercaptoethanol. We stored the tubes at −70° C. until cDNA synthesis. Total RNA was extracted by incubation with 2.2× volume of RNAclean XP beads (Agencourt) for 10 min at room temperature before tubes were placed on a magnet (DynaMag-2, Invitrogen) and washed three times with 80% ethanol. We allowed the beads to dry while still on magnet and eluted in H₂O. A modified SMART protocol (Quigley, M. F. et al., Unbiased molecular analysis of T cell receptor expression using template-switch anchored RT-PCR. Curr Protoc Immunol. 2011, Chapter 10:Unit10 33, herein incorporated by reference) was used for first-strand cDNA synthesis. The eluted RNA was transferred to RT1 mix (20 mM Tris-HCl pH 8, 0.2% Tween-20, 1 mM dNTP, 2 μM oligo d(T), 1 U/μl RNase Inhibitor) in total volume of 20 μl and incubated at 72° C. for 3 min followed by 1 min on ice. To complete cDNA synthesis, we added equal volume of the RT2 mix (1×FS buffer, 0.8 M Betaine, 6 mM MgCl2, 2.5 mM DTT, 2 μM TSO (5′-Bio-AAGCAGTGGTATCAACGCAGAGTACrGrGrG-3′), 1 U/μl RNase Inhibitor, 10 U/μl SuperScript II). The cDNA synthesis was carried out at 42° C. for 90 min followed by 15 min at 72° C. Subsequently, TRA and TRB genes were amplified in two rounds of semi-nested PCR reactions. The cDNA from each sample was divided into 3-6 replicates and amplified with indexed primers. The reaction mix for the first PCR was: 2 μl cDNA template, 200/40 nM forward primer mix (STRT-fwd S/L), 200 nM reverse primer (TRAC_rev1 or TRBC_rev1) with KAPA HiFi HotStart ReadyMix in a total volume of 20 μl. Amplified was performed by touchdown PCR to increase specificity. The cycling conditions were: 3 min at 95° C. followed by 5 cycles (15 s at 98° C., 60 s at 72° C.), 5 cycles (15 s at 98° C., 30 s at 70° C., 40 s at 72° C.) and 8 cycles (15 s at 98° C., 30 s at 65° C., 40 s at 72° C.). The second PCR was done in a total volume of 10 μl with 1 μl of first PCR product, 200 nM indexed forward primers (R2_STRT_In01-12), 200 nM barcoded reverse primers (TRAC 01-10_rev2 or TRBC_01-10_rev2) and KAPA HiFi HotStart ReadyMix for 2 min at 95° C. followed by 10 cycles (20 s at 98° C., 30 s at 65° C., 40 s at 72° C.) with final elongation at 72° C. for 5 min. A final third PCR reaction was carried out in a total volume of 20 μl with 2 μl of second PCR product, 200 nM forward primer (Illumina Seq Primer R2), 200 nM reverse primer (Illumina Seq Primer R1) and KAPA HiFi HotStart ReadyMix to prepare the sequencing library for the Illumina MiSeq platform. The cycling conditions were: 2 min at 95° C. followed by 15 cycles (20 s at 98° C., 30 s at 60° C., 40 s at 72° C.) with final elongation at 72° C. for 5 min. The PCR products were pooled, cleaned and concentrated with Ampure XP beads (Agencourt) or QIAquick PCR purification kit prior to gel extraction and cleaned with QIAquick Gel Extraction kit and QIAquick PCR purification kit (Qiagen). All primer sequences are listed in Table 4, below. The sequencing was done on an Illumina MiSeq sequencing platform using the 250 bp pair-end sequencing kit.

TABLE 4

| | |
|---|---|
| Oligo | Barcode Sequence (5′-3′) |

1^{st} PCR

| | |
|---|---|
| fwdS | Bio-CTAATACGACTCACTATAGGGC |
| fwdL | Bio-CTAATACGACTCACTATAGGGCAAGCAGTGGTATCAACGCAGAGT |
| TRAC_rev1 | GGAACTTTCTGGGCTGGGGAAGAAGGTGTCTTCTGG |
| TRBC_rev1 | TGCTTCTGATGGCTCAAACACAGCGACCT |

2^{nd} PCR fwd Replica barcode

| | |
|---|---|
| R2_bulk01 | ATGAGC GGCATTCCTGCTGAACCGCTCTTCCGATCTNNNNNNATGAGCAAGCAGTGGTATCAACGCAGAGT |
| R2_bu1k02 | CAACTA GGCATTCCTGCTGAACCGCTCTTCCGATCTNNNNNNCAACTAAAGCAGTGGTATCAACGCAGAGT |
| R2_bulk03 | CTAGCT GGCATTCCTGCTGAACCGCTCTTCCGATCTNNNNNNCTAGCTAAGCAGTGGTATCAACGCAGAGT |
| R2_bulk04 | ACTTGA GGCATTCCTGCTGAACCGCTCTTCCGATCTNNNNNNACTTGAAAGCAGTGGTATCAACGCAGAGT |
| R2_bulk05 | CACTCA GGCATTCCTGCTGAACCGCTCTTCCGATCTNNNNNNCACTCAAAGCAGTGGTATCAACGCAGAGT |
| R2_bu1k06 | TACAGC GGCATTCCTGCTGAACCGCTCTTCCGATCTNNNNNNTACAGCAAGCAGTGGTATCAACGCAGAGT |
| R2_bulk07 | CGTGAT GGCATTCCTGCTGAACCGCTCTTCCGATCTNNNNNNCGTGATAAGCAGTGGTATCAACGCAGAGT |
| R2_bulk08 | CACTGT GGCATTCCTGCTGAACCGCTCTTCCGATCTNNNNNNCACTGTAAGCAGTGGTATCAACGCAGAGT |
| R2_bulk09 | TGGTCA GGCATTCCTGCTGAACCGCTCTTCCGATCTNNNNNNTGGTCAAAGCAGTGGTATCAACGCAGAGT |
| R2_bulk10 | ATTGGC GGCATTCCTGCTGAACCGCTCTTCCGATCTNNNNNNATTGGCAAGCAGTGGTATCAACGCAGAGT |
| R2_bulk11 | TACAAG GGCATTCCTGCTGAACCGCTCTTCCGATCTNNNNNNTACAAGAAGCAGTGGTATCAACGCAGAGT |
| R2_bulk12 | GGAACT GGCATTCCTGCTGAACCGCTCTTCCGATCTNNNNNNGGAACTAAGCAGTGGTATCAACGCAGAGT |

2^{nd} PCR rev Sample barcode

| | |
|---|---|
| TRAC01_rev2 | ACCGTA ACACTCTTTCCCTACACGACGCTCTTCCGATCTNNNNNNACCGTACAGCTGGTACACGGCAGGGT |
| TRAC02_rev2 | GAGTAG ACACTCTTTCCCTACACGACGCTCTTCCGATCTNNNNNNGAGTAGCAGCTGGTACACGGCAGGGT |
| TRAC03_rev2 | TTACGC ACACTCTTTCCCTACACGACGCTCTTCCGATCTNNNNNNTTACGCCAGCTGGTACACGGCAGGGT |
| TRAC04_rev2 | CGTACT ACACTCTTTCCCTACACGACGCTCTTCCGATCTNNNNNNCGTACTCAGCTGGTACACGGCAGGGT |
| TRAC05_rev2 | GTGAAA ACACTCTTTCCCTACACGACGCTCTTCCGATCTNNNNNNGTGAAACAGCTGGTACACGGCAGGGT |
| TRAC06_rev2 | TAGCTT ACACTCTTTCCCTACACGACGCTCTTCCGATCTNNNNNNTAGCTTCAGCTGGTACACGGCAGGGT |
| TRAC07_rev2 | ACTGAT ACACTCTTTCCCTACACGACGCTCTTCCGATCTNNNNNNACTGATCAGCTGGTACACGGCAGGGT |
| TRAC08_rev2 | CCGTCC ACACTCTTTCCCTACACGACGCTCTTCCGATCTNNNNNNCCGTCCCAGCTGGTACACGGCAGGGT |
| TRAC09_rev2 | GGCTAC ACACTCTTTCCCTACACGACGCTCTTCCGATCTNNNNNNGGCTACCAGCTGGTACACGGCAGGGT |
| TRAC10_rev2 | ATTCCT ACACTCTTTCCCTACACGACGCTCTTCCGATCTNNNNNNATTCCTCAGCTGGTACACGGCAGGGT |
| TRBC01_rev2 | ATCTCG ACACTCTTTCCCTACACGACGCTCTTCCGATCTNNNNNNATCTCGCGACCTCGGGTGGGAACAC |
| TRBC02_rev2 | CAGATC ACACTCTTTCCCTACACGACGCTCTTCCGATCTNNNNNNCAGATCCGACCTCGGGTGGGAACAC |
| TRBC03_rev2 | TGACGA ACACTCTTTCCCTACACGACGCTCTTCCGATCTNNNNNNTGACGACGACCTCGGGTGGGAACAC |
| TRBC04_rev2 | GCTGAT ACACTCTTTCCCTACACGACGCTCTTCCGATCTNNNNNNGCTGATCGACCTCGGGTGGGAACAC |
| TRBC05_rev2 | CGATGT ACACTCTTTCCCTACACGACGCTCTTCCGATCTNNNNNNCGATGTCGACCTCGGGTGGGAACAC |
| TRBC06_rev2 | ACCACA ACACTCTTTCCCTACACGACGCTCTTCCGATCTNNNNNNACCACACGACCTCGGGTGGGAACAC |
| TRBC07_rev2 | GATCAG ACACTCTTTCCCTACACGACGCTCTTCCGATCTNNNNNNGATCAGCGACCTCGGGTGGGAACAC |
| TRBC08_rev2 | TCGGTC ACACTCTTTCCCTACACGACGCTCTTCCGATCTNNNNNNTCGGTCCGACCTCGGGTGGGAACAC |
| TRBC09_rev2 | GTCTGC ACACTCTTTCCCTACACGACGCTCTTCCGATCTNNNNNNGTCTGCCGACCTCGGGTGGGAACAC |
| TRBC10_rev2 | AGTCAA ACACTCTTTCCCTACACGACGCTCTTCCGATCTNNNNNNAGTCAACGACCTCGGGTGGGAACAC |

3rd PCR

| | |
|---|---|
| R1 | AATGATACGGCGACCACCGAGATCTACACTCTTTCCCTACACGACGCTCTTCCGATC |
| R2 | CAAGCAGAAGACGGCATACGAGATCGGTCTCGGCATTCCTGCTGAACCGCTC |

### Data Processing and Analysis

Raw reads from Illumina NGS were processed in a multistep pipeline. Single-cell TCR sequencing data was first pre-processed by using selected steps of the pRESTO toolkit (Vander Heiden J. A. et al., Bioinformatics 30(13):1930-1932, 2014, herein incorporated by reference). First, low-quality reads with average Phred quality score Q<30 were removed. Sequences were then unmasked according to barcodes (row, plate and column) and gene-specific primers (TRA/TRB), which were then annotated in the read header. Reads without recognisable primer sequences were removed. Subsequently, forward (R2) and reverse (R1) reads were paired according to Illumina coordinates and assembled into full-length TCR sequences. Next, identical duplicate sequences derived from the same cell were collapsed and the number of sequences collapsing as one sequence was denoted as “dupcount”. Only sequences with dupcount >2 were used for further analysis. In the last pre-processing step, we aligned the three highest ranking (in terms of dupcount) sequences on a per-cell, per-chain basis, implemented as a custom python script. Here, the highest-ranking sequence was aligned to the second highest ranking sequence using a dynamic programming algorithm (Needleman, S. B. & Wunsch, C. D., J Mol Biol. 48(3):443-453, 1970, herein incorporated by reference). For sequences aligning with <2% mismatches (relative to the length of the highest-ranking sequence, and ignoring gaps), the highest-ranking sequence was retained and the dupcounts were added up. Remaining sequences were discarded. Subsequently, the third-highest ranking sequence was aligned to the previous outcome, and possibly merged as well. Other pairs of the top three sequences were aligned as needed, always prioritising the highest-ranking sequence in terms of dupcounts.

Bulk-cell-derived sequencing data was pre-processed in much the same manner as pre-processing of single-cell sequencing data was performed, as described above. The difference was that sequences were marked according to barcoded gene-specific primers (TRA/TRB) in the R1 reads and the TSO sequence together with replicate barcodes in the R2 reads. The barcoded primers were then annotated in the read header.

We submitted pre-processed TCR sequences to the IMGT/HighV-QUEST online tool (Alamyar, E. et al., Methods Mol Biol. 882:569-604, 2012, herein incorporated by reference) for identification of V, D, J genes and alleles and the nucleotide sequences of the CDR3 junctions. Before analysing the IMGT/HighV-QUEST output, the IMGT annotation was parsed, stored in a relational database and subjected 6 to additional filters before extracting the sequences. This workflow was implemented as an in-house Java program together with a custom MySQL database. First, only productive sequences according IMGT annotation were included. For single-cell data, within each cell and each chain, duplicate sequences that had identical V genes, J genes and nucleotide CDR3 sequences were collapsed. Next, only valid singleton cells containing single TRA and TRB and dual TRA or TRB (maximum 3 chains) with dupcount >100 were considered for downstream analysis. Within samples taken from the same individual, cells were defined as belonging to the same clonotype when they shared identical V and J genes (subgroup level) in addition to identical nucleotide CDR3 regions for both the TRA and TRB genes. All bulk samples were divided after cDNA synthesis and amplified in independent PCR reactions that were barcoded with 3-6 replicate indices. Within each bulk TCR sample replicate, duplicate sequences defined as identical V genes, J genes and allowing for one nucleotide mismatch in CDR3 regions to account for PCR and sequencing errors were collapsed. Only sequences present in >2 distinct replicas and cumulative dupcount >10 were used for downstream analysis.

To assess data quality with regard to cross-contamination due to sample contamination or errors, we searched for identical paired TCRαβ nucleotide sequences across individuals in our single-cell data. Of a total of 3834 single cells expressing 1859 unique TCRαβ clonotypes, we found four paired TCRαβ nucleotide sequences that were identical across individuals. In every case, samples sharing the same sequences were prepared and sequenced in different libraries. Similarly, in our bulk sequencing data, we found 12 TCRβ sequences that were identical across individuals out of a total of 1129 unique TCRβ sequences. Of these, 9 sequences were found in different libraries. Overall, shared nucleotide sequences across patients were found in approximately 1% of all sequences when clonotype was defined by TCRβ nucleotide sequence alone. When clonotype was defined by paired TCRαβ nucleotide sequences, sharing across patients was found in 0.2% of the clonotypes demonstrating that cross-contamination is not an issue.

### Statistics

Repertoire diversity was quantified in samples with >20 cells with a non-parametric estimate of the classic Shannon entropy where corrections were made for under-sampling by taking into account the unseen species (clonotypes) in the samples. This sample-corrected version of Shannon diversity index performs largely independently of sample sizes.

### Example 1: General Methods

a. Sample collection. 8-18 ml blood samples are taken by venipuncture in ACD or EDTA anti-coagulated tubes. Blood samples are stored and transported at room temperature until processing, which takes place within 48 hours.
b. Sample processing to yield PBMC. Blood samples are processed by gradient centrifugation or similar methods to yield peripheral blood mononuclear cells (PBMC).
c. Optional: enrichment of effector memory CD4+ T-cells. PBMC are enriched for effector memory CD4+ T-cells by negative selection with commercial kits (Miltenyi). Typically around 2 million effector memory CD4+ T-cells from 18 ml of blood are used per individual.
d. Storage of samples. Cells from steps 2 and/or 3 are pelleted and kept at −80° C. until processed.
e. mRNA extraction, cDNA synthesis and PCR amplification for TCRα and TCRβ genes. mRNA is extracted using an RNA extraction kit (Qiagen RNAeasy mini kit or similar). First-strand cDNA is synthesised using an oligo-dT reverse primer together with a TSO (Template-Switching Oligo). Multiple rounds of PCR will amplify TCRα and TCRβ genes by using specific reverse primers and a universal forward primer annealing to the PCR handle introduced by the TSO. UMI ((Unique Molecular Identifier; optional), replicate barcodes and sample indices and Illumina sequencing adaptors are also added during the same PCR reactions.
f. Alternative strategy. In place of mRNA, genomic DNA (gDNA) can be extracted for the same samples. TCR genes are then specifically amplified by using V-gene-specific forward (multiple, one for each of the V gene segments) and J-gene-specific (multiple, one for each of the J gene segments) reverse primers. A sequencing-ready library is then made by adding platform-compatible adaptors.
g. Sequencing. Prepared libraries are sequenced on an Illumina HiSeq platform with 150 bp PE kits. Typical sequencing depth is ˜20 million reads per patient amounting to ˜5× sequencing depth per unique TCR gene.
h. Sequencing data processing and identification of TCR sequences. Sequencing data is processed by quality filter, index and barcode identification, UMI identification and analysed for TCR use (by V-QUEST engine on IMGT.org, MiXCR software package or similar). Data is further quality-assessed to remove errors introduced by PCR and/or sequencing.
i. Scoring of TCR dataset from each individual for the presence or absence of defined known public celiac disease-specific TCR sequences (specific sequences in short). The presence of a particular specific sequence or a sequence motif that is common to many specific sequences will result in a score for the individual TCR dataset. The score quantitatively determined according to the number of times the particular sequences are observed in the dataset (1 replicate versus several replicates, few UMI versus many UMI, number of clonotypes as estimated by MiXCR). The score is then normalised for sequencing depth and library size by dividing by total number of reads, total number of clonotypes observed or total number of cells sequenced.
j. Celiac disease diagnostic evaluation based on the normalised TCR score. Finally, based on the cumulative normalised score for the presence of all known specific TCR sequences or motifs, each dataset will be evaluated to be likely derived from a celiac disease patient or not.

### Example 2: TCR Sequencing of Effector Memory CD4+ T-Cells from Blood

### Study Design

Since gluten-specific T-cells will be activated and divide as a result of gluten stimulation in celiac disease patients, the disease-specific T-cells are found as expanded clones within the effector memory compartment of CD4+ T-cells in blood. Therefore, we have isolated the effector memory fraction of CD4+ T-cells from PBMC and subjected it to unbiased PCR amplification and sequencing. The minimum number of effector memory CD4+ T-cells subjected to sequencing per sample is 500 000 and the optimal number is at least 2 million cells.

### Data Analysis

The sequencing data from HiSeq platform is de-multiplexed for sample barcodes, and the TCR sequences are retrieved by the software package MiXCR. This software package assigns a clonotype count estimate for each nucleotide TCR sequence based on the number of reads.

Since we expect that the gluten-specific TCR sequences are clonally expanded, i.e. many cells carry these TCR sequences, as a result of gluten stimulation in celiac disease patients, we summarise the clonotype counts as estimated by the MiXCR software that are represented by at least one of the public gluten-specific TCR sequences. The data is matched against total 377 public gluten-specific TCR sequences (SEQ ID NOs: 1-377). Only complete identical amino acid sequences were scored. The total number of clonotype counts including any of the given 377 public gluten-specific TCR sequences was then divided by the total number of TCR reads in the sequenced sample as estimated by MiXCR, in order to normalise for variable sample sizes. That normalised number is shown as number of nucleotide sequences which contribute to the score per million reads.

### Results

In a limited dataset of blood samples from 4 untreated celiac disease patients and 4 healthy controls, we found that the normalised number of sequences which contribute to the score is higher in all 4 patient samples compared with all 4 control samples (see Table 5).

If the previously published TRBV7-2/7-3_ASSxRxTDTQY_TRBJ2-3 sequences were excluded from the public TCR sequence list, one of the celiac disease sample (CD1416) returned a very low value whereas the other 3 patient samples all scored higher than all 4 control samples. To note, the CD1416 patient sample contained much less total TCR sequences compared to all the other samples in this dataset. We believe that this sample size limitation is the major cause of failure to detect public gluten-specific TCR sequences other than the published TRBV7-2/7-3_ASSxRxTDTQY_TRBJ2-3 sequence.

TABLE 5

| | Celiac | R-motif, | R-motif, | Other | | |
|---|---|---|---|---|---|---|
| Donor ID | disease | BV7-2 | BV7-3 | sequences | Sum | Rank |
| cd1416 | yes | 2 470 | — | 0 | 2 470 | 1 |
| cd1424 | yes | 203 | 3 | 295 | 501 | 2 |
| cd1421 | yes | 69 | 15 | 256 | 340 | 3 |
| cd1423 | yes | 52 | — | 188 | 240 | 4 |
| cd1234 | no | 74 | 6 | 150 | 230 | 5 |
| cd1365 | no | 46 | 54 | 94 | 194 | 6 |
| cd1363 | no | 12 | 2 | 155 | 170 | 7 |
| cd1425 | no | 22 | — | 145 | 166 | 8 |

“R-motif, BV7-2” indicates TCR sequences with the consensus TRBV7-2_ASSxRxTDTQY_TRBJ2-3. “R-motif, BV7-3” indicates TCR sequences with the consensus TRBV7-3_ASSxRxTDTQY_TRBJ2-3. “Other sequences denotes” all 377 public gluten-specific TCR sequences (SEQ ID NOs: 1-377) excluding those that match the “R-motif, BV7-2” or “R-motif, BV7-3”. “Sum” indicates all 377 public gluten-specific TCR sequences (SEQ ID NOs: 1-377).

### Example 3: General Methods for Biopsy-Based Test

1. Sample collection. Biopsies are taken from the descending duodenum by gastroendoscopic procedures. Biopsy samples are transported in RPMI buffer on ice.
2. Sample processing to yield lamina propria cells in suspension. Biopsy samples are incubated with EDTA solution to remove the epithelia including intra-epithelial lymphocytes. Biopsy samples are digested with collagenase (or alternative enzymes that digest tissue). Cells in suspension are filtered and counted.
3. Optional: enrichment of CD4+ T cells. Lamina propria cells are enriched for CD4+ T cells by positive selection with commercial kits (Miltenyi).
4. Lysis of cells in replicate wells in different dilutions. Cells from steps 2 and/or 3 are added to storage buffer (TCL buffer from Qiagen, PBS or similar). Cells from each subject are distributed in different dilutions (starting from 108 000 lamina propria cells or 1 080 CD4+ T cells per well) and in replicates (up to 8). In total cells from 1-3 biopsies are used per individual.
5. mRNA extraction, cDNA synthesis and PCR amplification for TCRα and TCRβ genes. mRNA is extraction from the cell lysates by RNA extraction kit (Qiagen RNAeasy mini kit), immobilised poly-dT oligos (TurboCapture kit from Qiagen), or RNA extraction beads (RNAcleanup XP Agencourt® beads). First-strand cDNA is synthesised by using oligo-dT reverse primer together with a TSO (Template-Switching Oligo). Multiple rounds of semi-nested PCR will amplify TCRα and TCRβ genes by using gene-specific reverse primers and forward universal PCR handle primer introduced by TSO. UMI (Unique Molecular Identifier), replicate barcode, sample indices and Illumina sequencing adaptors are also added during the same PCR reactions.
6. Sequencing. Prepared libraries are sequenced on Illumina MiSeq platform with 250 bp or 300 bp PE kits. Typical sequencing depth is 1-2 million reads per individual.
7. Sequencing data processing and identification of TCR sequences. Sequencing data is processed by quality filter, index and barcode identification, UMI identification and analysed for TCR use (by V-QUEST engine on IMGT.org, MiTCR software package or similar). Data is further quality-assessed to remove errors introduced by PCR and/or sequencing (pRESTO or similar software).
8. Scoring of TCR dataset from each individual for the presence or absence of defined known public celiac disease-specific TCR sequences (specific sequences in short). The presence of a particular specific sequence or a sequence motif that is common to many specific sequences will give a score for the individual TCR dataset. The score is quantitative according to the number of times the particular sequences are observed in the dataset (1 replicate versus several replicates, few UMI versus many UMI).
9. Celiac disease diagnostic evaluation based on the TCR score. Finally, based on the cumulative score for the presence of all known specific TCR sequences or motifs, each dataset will be evaluated to be likely derived from a celiac disease patient or not. The evaluation may be adjusted according to variable sequence depth and coverage.

### Example 4: TCR Sequencing of Unfractionated Lamina Propria Samples

In small intestinal lamina propria, the prevalence of gluten-specific T-cells in celiac disease patients who consume gluten is believed to be around 2%. Thus, we have used this material to prove that we can differentiate celiac disease patients from healthy controls by the presence of TCR sequences that are known to be gluten-specific and public, i.e. shared by several individuals.

### Study Design

1.3×10⁶ lamina propria cells obtained by enzymatic digestion of 1-2 duodenal biopsies were plated out in 32 wells at four different dilutions. After unbiased PCR amplification and sequencing, the resulting sequencing results were mapped by sample and well barcodes, and the TCR information is retrieved by the online software package IMGT. Since a minimum number of TCR sequences is needed in the sample for meaningful downstream analysis, we have excluded samples that due to technical reasons contained less than 100 000 productive sequencing reads. Productive sequencing reads are defined as reads that resulted in productive TCR sequences.

### Data Analysis

TCR amino acid sequences were then compared with a list of 229 public gluten-specific TCR sequences found in a study including 17 HLA-DQ2.5+ celiac disease patients (the sequences set forth in SEQ ID NOs: 1, 2, 4-15, 17, 18, 20-25, 27-37, 39-48, 51, 53-55, 59, 60, 62, 64, 68, 69, 72-75, 77-79, 81-85, 87, 88, 90-92, 94, 96-105, 107, 108, 111, 112, 117-120, 122, 124, 127-129, 132, 133, 137-141, 143, 145, 151-153, 156, 157, 159, 163-165, 168-171, 173, 176-179, 182, 184, 185, 188-190, 194-196, 198, 199, 201, 202, 204-206, 209-211, 213, 214, 218-218, 220, 223-225, 228, 230, 232-234, 238, 241-250, 252, 253, 255, 258-263, 265, 266, 270, 271, 275-277, 283, 290-292, 294, 296, 297, 299-301, 303-309, 312, 314, 316, 318, 319, 322, 324, 330, 331, 333, 336, 339, 341, 342, 344, 346, 349, 350, 352, 358-360, 366, 367 and 369-375). Since we have observed that TCR sequences that differ by a few amino acids in the CDR3 region can all be gluten-specific, we have counted TCR sequences in the test material that are either completely identical or differ by one amino acid with the reference gluten-specific TCR sequences. Identical sequences were scored 4 and those that differ by one amino acid were scored 3. If the same TCR sequence was observed in multiple wells in the same sample, these were counted independently. Finally, the total score was adjusted to sequencing library size and normalised to per 100 000 productive reads.

### Results

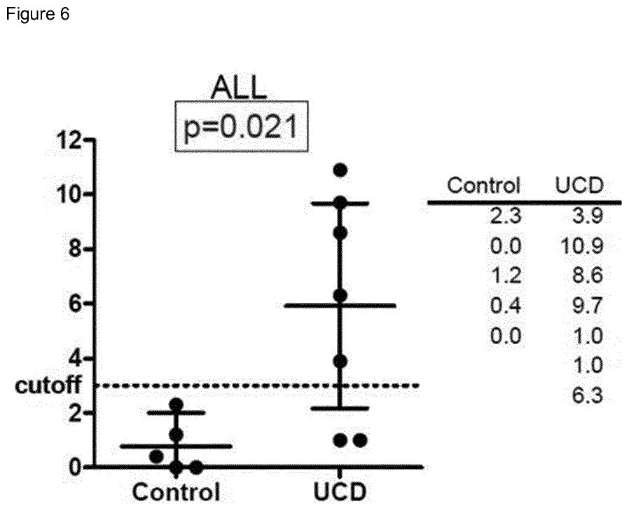

When scoring for the presence of all 229 public gluten-specific TCR sequences, we found that the library size-adjusted score is significantly higher (p=0.021) in the untreated celiac disease patient group (n=7) compared to the control group (n=5). Moreover, all 5 control subjects had adjusted scores of 3 or less whereas 5 of 7 individuals in the patient groups had scores above this threshold value (FIG. 6).

The results were similar (p=0.017) when the same data were scored for the presence of all the above-mentioned public gluten-specific TCR sequences except the well-known TRBV7-2/7-3_ASSxRxTDTQY_TRBJ2-3 (x denotes any amino acid) public gluten-specific TCR sequences that had been published earlier.

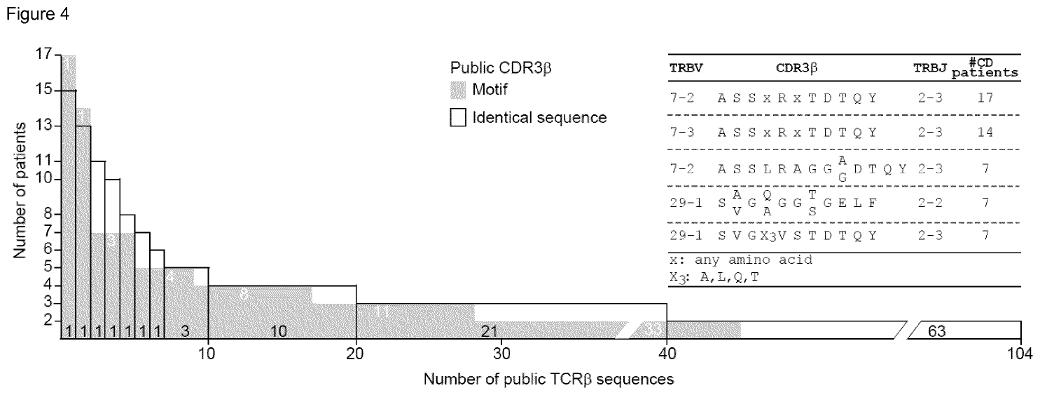

Indeed, when the top five gluten-specific TRB motifs as listed in FIG. 4 were removed from the analysis, the results remained the same (p=0.010) indicating that the test is robust and is not dependent on a few top-score sequences.

### Example 5: Larger Scale Diagnostic Trial

### Study Design

The study design was essentially the same as for Example 4, except a larger cohort of 17 subjects were included in the study. All subjects were HLA-DQ2.5+. The 17 subjects consisted of 6 healthy controls, 10 patients previously diagnosed with celiac disease and one individual with “potential celiac disease”.

The term “potential celiac disease” is used to describe individuals who produce disease-associated gluten-specific antibodies at levels detectable in serological tests, but who upon histological examination of small intestinal biopsies are found not to have sufficient tissue damage to fulfil the criteria for celiac disease diagnosis. Many individuals with potential celiac disease are subsequently diagnosed with full celiac disease, though progression of the condition to full celiac disease can take some years.

### Methods

DNA samples were obtained and sequencing performed as described above. Patient libraries were analysed for the presence of all TCRβ chain sequences presented in Tables 1 to 3. Matched sequencing reads were called when a read encoded an identical CDR3 amino acid sequence and utilised the identical V gene segment to any one of the TCRβ chains set forth in Tables 1 to 3. A normalised score was obtained for each patient library by dividing the number of matched reads by the total read count, i.e. determining the proportion of total reads that were matched.

The threshold was selected as a normalised score of 0.187% (i.e. 0.187 permille, or 0.187 matched reads per thousand total reads). This threshold was selected to maximise total accuracy (i.e. to yield the minimum total number of false positives and false negatives). Since the threshold selection in this example is performed based on a priori knowledge of the celiac status of each subject, it corresponds to a calibration procedure for threshold selection.

### Results

The results of the diagnostic analysis are presented in the table below. Correctly assigned results based on the threshold are shown in bold in the right-hand columns. “Yes” for celiac status indicates the presence of celiac disease; “no” indicates the absence of celiac disease.

| | | | | Predicted | Known |
|---|---|---|---|---|---|
| | Donor | | Normalized | celiac | celiac |
| Rank | ID | Score | score (%) | status | status |
| 1 | 1416 | 16 541 | 2.472 | Yes | Yes |
| 2 | 1454 | 2 143 | 0.877 | Yes | Yes |
| 3 | 1508 | 2 004 | 0.865 | Yes | Yes |
| 4 | 1451 | 1 417 | 0.580 | Yes | Yes |
| 5 | 1424 | 2 419 | 0.451 | Yes | Potential |
| 6 | 1438 | 836 | 0.389 | Yes | Yes |
| 7 | 1421 | 2 040 | 0.355 | Yes | Yes |
| 8 | 1425 | 1 862 | 0.340 | Yes | No |
| 9 | 1441 | 686 | 0.255 | Yes | Yes |
| 10 | 1365 | 1 336 | 0.212 | Yes | No |
| 11 | 1516 | 432 | 0.211 | Yes | Yes |
| 12 | 1423 | 1 007 | 0.187 | Yes | Yes |
| 13 | 1234 | 1 180 | 0.186 | No | No |
| 14 | 1450 | 350 | 0.168 | No | No |
| 15 | 1363 | 748 | 0.155 | No | No |
| 16 | 1434 | 179 | 0.091 | No | Yes |
| 17 | 1461 | 183 | 0.081 | No | No |

The above results provide a sensitivity of 91% (10/11 celiac patients correctly diagnosed, including the subject with potential celiac disease) and a specificity of 67% ( 4/6 subjects who do not suffer from celiac disease were correctly identified as such).

1. An in vitro method for diagnosing celiac disease in a human subject or monitoring the response of a human subject to treatment therefor, said method comprising the steps:
a) isolating nucleic acids from a sample obtained from the subject, wherein said sample comprises T-cells;
b) sequencing nucleotide sequences which encode TCRα chains and nucleotide sequences which encode TCRβ chains to provide a TCR dataset;
c) assigning a score to the TCR dataset, wherein said score is determined by the abundance in the dataset of nucleotide sequences which encode at least two TCRα or TCRβ amino acid sequences, wherein said at least two TCRα or TCRβ amino acid sequences comprise:
(i) at least one TCRα or TCRβ amino acid sequence selected from SEQ ID NOs: 1 to 50; and
(ii) at least one TCRα or TCRβ amino acid sequence selected from SEQ ID NOs: 51 to 432;
d) normalising said score to provide a normalised score representative of:
(i) the frequency of the nucleotide sequences in the TCR dataset; or
(ii) the frequency of T-cells expressing the nucleotide sequences in the sample; and
e) comparing said normalised score to a defined threshold, wherein the subject is diagnosed with celiac disease if said normalised score is equal to or higher than the defined threshold, or the response to treatment is determined by comparison to the defined threshold.

2. The method of claim 1, wherein said sample is a blood sample.

3. The method of claim 2, wherein peripheral blood mononuclear cells (PBMC) are isolated from said blood sample, and the isolation of nucleic acids of step (a) is performed on said isolated PBMC.

4. The method of any one of claims 1 to 3, wherein the sample is enriched for CD4+ effector memory T-cells.

5. The method of any one of claims 1 to 4, wherein mRNA is isolated from the sample and reverse transcribed into cDNA, and the sequencing of part (b) is performed on the cDNA.

6. The method of any one of claims 1 to 4, wherein gDNA is isolated from the sample, and the sequencing of part (b) is performed on the gDNA.

7. The method of claim 5 or 6, wherein nucleotide sequences which encode all the TCRα chains and TCRβ chains in the samples are amplified, yielding a library of amplification products, and said library is sequenced.

8. The method of claim 5 or 6, wherein the nucleotide sequences which encode the TCRα chains and TCRβ chains are amplified using a composition suitable for multiplex PCR comprising a plurality of nucleic acid primers, wherein the composition comprises primers able to specifically hybridise to the TCR V-gene segments specified in Table 1 and Table 2 and primers able to specifically hybridize to the TCR J-gene segments specified in Table 1 and Table 2, wherein an amplification product may be obtained using a combination of a primer able to specifically hybridise to a TCR V-gene segment and a primer able to specifically hybridise to a TCR J-gene segment.

9. The method of claim 5 or 6, wherein the nucleotide sequences which encode the TCRα chains and TCRβ chains are amplified using a composition suitable for multiplex PCR comprising a plurality of nucleic acid primers, wherein the composition comprises primers able to specifically hybridize to the TCR V-gene segments specified in Table 1 and Table 2 and primers able to specifically hybridise to a nucleotide sequence encoding a TCR constant region, wherein an amplification product may be obtained using a combination of a primer able to specifically hybridise to a TCR V-gene segment and a primer able to specifically hybridise to a nucleotide sequence encoding a TCR constant region.

10. The method of any one of claims 1 to 9, wherein said score is determined by the abundance in the dataset of nucleotide sequences which encode at least 50 TCRα and/or TCRβ amino acid sequences selected from SEQ ID NOs: 1 to 377.

11. The method of claim 10, wherein said score is determined by the abundance in the dataset of nucleotide sequences which encode at least 100 TCRα and/or TCRβ amino acid sequences selected from SEQ ID NOs: 1 to 377.

12. The method of claim 11, wherein said score is determined by the abundance in the dataset of nucleotide sequences which encode at least 200 TCRα and/or TCRβ amino acid sequences selected from SEQ ID NOs: 1 to 377.

13. The method of claim 12, wherein said score is determined by the abundance in the dataset of nucleotide sequences which encode at least the 229 TCRα and TCRβ amino acid sequences set forth in SEQ ID NOs: 1, 2, 4-15, 17, 18, 20-25, 27-37, 39-48, 51, 53-55, 59, 60, 62, 64, 68, 69, 72-75, 77-79, 81-85, 87, 88, 90-92, 94, 96-105, 107, 108, 111, 112, 117-120, 122, 124, 127-129, 132, 133, 137-141, 143, 145, 151-153, 156, 157, 159, 163-165, 168-171, 173, 176-179, 182, 184, 185, 188-190, 194-196, 198, 199, 201, 202, 204-206, 209-211, 213, 214, 218-218, 220, 223-225, 228, 230, 232-234, 238, 241-250, 252, 253, 255, 258-263, 265, 266, 270, 271, 275-277, 283, 290-292, 294, 296, 297, 299-301, 303-309, 312, 314, 316, 318, 319, 322, 324, 330, 331, 333, 336, 339, 341, 342, 344, 346, 349, 350, 352, 358-360, 366, 367 and 369-375.

14. The method of claim 12 or 13, wherein said score is determined by the abundance in the dataset of nucleotide sequences which encode at least 300 TCRα and/or TCRβ amino acid sequences selected from SEQ ID NOs: 1 to 377.

15. The method of claim 14, wherein said score is determined by the abundance in the dataset of nucleotide sequences which encode the TCRα and TCRβ amino acid sequences set out in SEQ ID NOs: 1 to 377.

16. The method of any one of claims 1 to 9, wherein said score is determined by the abundance in the dataset of nucleotide sequences which encode at least 300 TCRα and/or TCRβ amino acid sequences selected from SEQ ID NOs: 1 to 432.

17. The method of any one of claims 1 to 16, wherein said normalised score is the frequency in the sample of T-cells which express a TCRα chain or TCRβ chain encoded by a nucleotide sequence which contributes to the score.

18. The method of any one of claims 1 to 16, wherein said normalised score is the frequency in the TCR dataset of T-cell clonotypes which express a TCRα chain or TCRβ chain encoded by a nucleotide sequence which contributes to the score.

19. The method of any one of claims 1 to 16, wherein said normalised score is the frequency in the TCR dataset of nucleotide sequences which contribute to the score.

20. The method of claim 19, wherein the defined threshold is at least 240 nucleotide sequences which contribute to the score per million reads.

21. The method of claim 20, wherein the defined threshold is at least 300 nucleotide sequences which contribute to the score per million reads.

22. The method of claim 21, wherein the defined threshold is at least 400 nucleotide sequences which contribute to the score per million reads.

23. The method of any one of claims 1 to 19, wherein said method is for monitoring the response of a subject to treatment for celiac disease, and the defined threshold is the normalised score of the subject prior to the initiation of treatment.

24. A composition suitable for multiplex PCR comprising a plurality of nucleic acid primers, wherein the composition comprises:
(i) primers able to specifically hybridise to the TCR V-gene segments specified in Table 1 and Table 2; and
(ii) primers able to specifically hybridise to the TCR J-gene segments specified in Table 1 and Table 2 or primers able to specifically hybridise to a nucleotide sequence encoding a TCR constant region;
wherein a primer of part (i) and a primer of part (ii) may be used in combination to generate an amplification product.
